SEQUENCE LISTING (42 sequences)

SEQ ID NO: 1 - DNA, 1666 bp, Homo sapiens
ggccccccgttccccgccaggctgcaggcgtcgggcctgggccgtcagggcagctgtgaccggatcgcttcccgggcggcgagctgggggtgcacccggaccgccgcccccgggatcatgggcaatggcatgaccaaggtacttcctggactctacctcggaaacttcattgatgccaaagacctggatcagctgggccgaaataagatcacacacatcatctctatccatgagtcaccccagcctctgctgcaggatatcacctaccttcgcatcccggtcgctgatacccctgaggtacccatcaaaaagcacttcaaagaatgtatcaacttcatccactgctgccgccttaatggggggaactgccttgtgcactgctttgcaggcatctctcgcagcaccacgattgtgacagcgtatgtgatgactgtgacggggctaggctggcgggacgtgcttgaagccatcaaggccaccaggcccatcgccaaccccaacccaggctttaggcagcagcttgaagagtttggctgggccagttcccagaagggtgccagacataggacctcaaaaacctctggtgcccaatgccctccgatgacttcagcaacctggatggtcaccggacccaaagtaccagatctgtctgtgcttcggtgaggaggacccgggccccacacagcaccccaaggagcagctcatcatggcggacgtgcaggtgcagcttcggcctgggagctcgtcctgcactctaagtgcctcaaccgagcgcccagatgggtcctcaacccctggcaaccccgatggcatcactcaccttcaatgcagctgcctccatcctaagcgagccgcttcctcttcttgtacccgctgaaggcagcccccaacaggggggctccctactcccacccaaccctgcccacactaagcccatagacttggggcctcccccggcacatcacccaggtctgccggacggcagaggtggatcgcggccttccactcctctgtcacggggccccggaactcgagagtaggccacaccgccccccagctgggcatggggcttcggcaggaaactgaacttgatcttgaggccccagaaaggcagcaactggagcagaagcaagacttcatctcttgctgacagcccaatttgtcaatagcgctttcctcagagccagccttaacctgctgttgagtccattaaaacgtttgcttaaagtttttaccaataattagatcatcagggttgtttagtgtgggatcaagccataacaaaactgcctagcctctcaggggcctagaatttacagaaccttcctcctccctgcagcaagtctctcttctttattctgggggctgggaaggatcccaaaacagggaacttggccgaaccctgggctttggatgctaaccactgaagtaccagcacctgtaggatgctgtctttgaagaaactgaggcggacctccaaatgcagccctaaggcagaggtcaacgtggaagaccagcccttctccaagccccactggtctttgcaagctgtacgttgtaggcaatctgagaactggaaagggggactacaaccagaaagttggttaccctgccatgggaataaagtagctgttttccaccccaaaaaaaaaaaaaaaaaaaaaaa

SEQ ID NO: 2 - protein, 181 aa, Homo sapiens
MGNGMTKVLPGLYLGNFIDAKDLDQLGRNKITHIISIHESPQPLLQDITYLRIPVADTPEVPIKKHFKECINFIHCCRLNGGNCLVHCFAGISRSTTIVTAYVMTVTGLGWRDVLEAIKATRPIANPNPGFRQQLEEFGWASSQKGARHRTSKTSGAQCPPMTSATWMVTGPKVPDLSVLR

SEQ ID NO: 3 - DNA, 1807 bp, Homo sapiens
ggccccccgttccccgccaggctgcaggcgtcgggcctgggccgtcagggcagctgtgaccggatcgcttcccgggcggcgagctgggggtgcacccggaccgccgcccccgggatcatgggcaatggcatgaccaaggtacttcctggactctacctcggaaacttcattgatgccaaagacctggatcagctgggccgaaataagatcacacacatcatctctatccatgagtcaccccagcctctgctgcaggatatcacctaccttcgcatcccggtcgctgatacccctgaggtacccatcaaaaagcacttcaaagaatgtatcaacttcatccactgctgccgccttaatggggggaactgccttgtgcactgctttgcaggcatctctcgcagcaccacgattgtgacagcgtatgtgatgactgtgacggggctaggctggcgggacgtgcttgaagccatcaaggccaccaggcccatcgccaaccccaacccaggctttaggcagcagcttgaagagtttggctgggccagttcccagaagggtgccagacataggacctcaaaaacctctggtgcccaatgccctccgatgacttcagcaacctgcctgctggctgcacgtgtggctcttctctccgcagcgctggtgcgcgaagccaccgggcgcacagcccagcgctgtcgtctgagtccgcgggcggccgccgagcgcctgctggggccgccacctcacgttgcagcaggatggtcaccggacccaaagtaccagatctgtctgtgcttcggtgaggaggacccgggccccacacagcaccccaaggagcagctcatcatggcggacgtgcaggtgcagcttcggcctgggagctcgtcctgcactctaagtgcctcaaccgagcgcccagatgggtcctcaacccctggcaaccccgatggcatcactcaccttcaatgcagctgcctccatcctaagcgagccgcttcctcttcttgtacccgctgaaggcagcccccaacaggggggctccctactcccacccaaccctgcccacactaagcccatagacttggggcctccccggcggcacatcacccaggtctgccggacggcagaggtggatcgcggccttccactcctctgtcacggggccccggaactcgagagtaggccacaccgccccccagctgggcatggggcttcggcaggaaactgaacttgatcttgaggccccagaaaggcagcaactggagcagaagcaagacttcatctcttgctgacagcccaatttgtcaatagcgctttcctcagagccagccttaacctgctgttgagtccattaaaacgtttgcttaaagtttttaccaataattagatcatcagggttgtttagtgtgggatcaagccataacaaaactgcctagcctctcaggggcctagaatttacagaaccttcctcctccctgcagctagtctctcttctttattctgggggctgggaaggatcccaaaacagggaacttggccgaaccctgggctttggatgctaaccactgaagtaccagcacctgtaggatgctgtctttgaagaaactgaggcggacctccaaatgcagccctaaggcagaggtcaacgtggaagaccagcccttctccaagccccactggtctttgcaagctgtacgttgtaggcaatctgagaactggaaagggggactacaaccagaaagttggttaccctgccatgggaataaagtagctgttttccaccccaaaaaaaaaaaaaaaaaaaaaaaaa

SEQ ID NO: 4 - protein, 298 aa, Homo sapiens
MGNGMTKVLPGLYLGNFIDAKDLDQLGRNKITHIISIHESPQPLLQDITYLRIPVADTPEVPIKKHFKECINFIHCCRLNGGNCLVHCFAGISRSTTIVTAYVMTVTGLGWRDVLEAIKATRPIANPNPGFRQQLEEFGWASSQKGARHRTSKTSGAQCPPMTSATCLLAARVALLSAALVREATGRTAQRCRLSPRAAAERLLGPPPHVAAGWSPDPKYQICLCFGEEDPGPTQHPKEQLIMADVQVQLRPGSSSCTLSASTERPDGSSTPGNPDGITHLQCSCLHPKRAASSSCTR

SEQ ID NO: 5 - DNA, 1268 bp, Homo sapiens
ggccccccgttccccgccaggctgcaggcgtcgggcctgggccgtcagggcagctgtgaccggatcgcttcccgggcggcgagctgggggtgcacccggaccgccgcccccgggatcatgggcaatggcatgaccaaggtacttcctggactctacctcggaaacttcattgatgccaaagacctggatcagctgggccgaaataagatcacacacatcatctctatccatgagtcaccccagcctctgctgcaggatatcacctaccttcgcatcccggtcgctgatacccctgaggtacccatcaaaaagcacttcaaagaatgtatcaacttcatccactgctgccgccttaatggggggaactgccttgtgcactgctttgcaggcatctctcgcagcaccacgattgtgacagcgtatgtgatgactgtgacggggctaggctggcgggacgtgcttgaagccatcaaggccaccaggcccatcgccaaccccaacccaggctttaggcagcagcttgaagagtttggctgggccagttcccagaagggtgccagacataggacctcaaaaacctctggtgcccaatgccctccgatgacttcagcaacctggatggtcaccggacccaaagtaccagatctgtctgtgcttcggtgaggaggacccgggccccacacagcaccccaaggagcagctcatcatggcggacgtgcaggtgcagcttcggcctgggagctcgtcctgcactctaagtgcctcaaccgagcgcccagatgggtcctcaacccctggcaaccccgatggcatcactcaccttcaatgcagcttgcctccatcctaagcgagccgcttcctcttcttgtacccgctgaaggcaagcccccaacaggggggctccctactcccacccaaccctgcccacactaagcccatagacttggggcctcccccggcacatcacccaggtctgccggacggcagaggtggatcgcggccttccactcctctgtcacggggccccggaactcgagagtaggcctcaccgccccccagctgggcatggggcttcggcaggaaactgaacttgatcttgaggccagcagaaaggcagcaactggagcagaagcaagacttcatctcttgctgacagcccaatttgtcaatagcgctttcctcagagccagccttaacctgctgttgagtccattaaaacgtttgcttaaagtttttaccaataaaaaaaaaaaaaaaaaaaaaaaaaa

SEQ ID NO: 6 - protein, 181 aa, Homo sapiens; identical to SEQ ID NO: 2

SEQ ID NO: 7 - DNA, 1045 bp, Homo sapiens
ggccccccgttccccgccaggctgcaggcgtcgggcctgggccgtcagggcagctgtgaccggatcgcttcccgggcggcgagctgggggtgcacccggaccgccgcccccgggatcatgggcaatggcatgaccaaggtacttcctggactctacctcggaaacttcattgatgccaaagacctggatcagctgggccgaaataagatcacacacatcatctctatccatgagtcaccccagcctctgctgcaggatatcacctaccttcgcatcccggtcgctgatacccctgaggtacccatcaaaaagcacttcaaagaatgtatcaacttcatccactgctgccgccttaatggggggaactgccttgtgcactgctttgcaggcatctctcgcagcaccacgattgtgacagcgtatgtgatgactgtgacggggctaggctggcgggacgtgcttgaagccatcaaggccaccaggcccatcgccaaccccaacccaggctttaggcagcagcttgaagagtttggctgggccagttcccagaagggtgccagacataggacctcaaaaacctctggtgcccaatgccctccgatgacttcagcaacctggatggtcaccggacccaaagtaccagatctgtctgtgcttcggtgaggaggacccgggccccacacagcaccccaaggagcagctcatcatggcggacctagtctctcttctttattctgggggctgggaaggatcccaaaacagggaacttggccgaaccctgggctttggatgctaaccactgaagtaccagcacctgtaggatgctgtctttgaagaaactgaggcggacctccaaatgcagccctaaggcagaggtcaacgtggaagaccagcccttctccaagccccactggtctttgcaagctgtacgttgtaggcaatctgagaactggaaagggggactacaaccagaaagttggttaccctgccatgggaataaagtagctgttttccaccccataaaaaaaaaaaaaaaaaaaaaaaa

SEQ ID NO: 8 - protein, 181 aa, Homo sapiens; identical to SEQ ID NO: 2

SEQ ID NO: 9 - DNA, 982 bp, Homo sapiens
ggccccccgttccccgccaggctgcaggcgtcgggcctgggccgtcagggcagctgtgaccggatcgcttcccgggcggcgagctgggggtgcacccggaccgccgcccccgggatcatgggcaatggcatgaccaaggtacttcctggactctacctcggaaacttcattgatgccaaagacctggatcagctgggccgaaataagatcacacacatcatctctatccatgagtcaccccagcctctgctgcaggatatcacctaccttcgcatcccggtcgctgatacccctgaggtacccatcaaaaagcacttcaaagaatgtatcaacttcatccactgctgccgccttaatggggggaactgccttgtgcactgctttgcaggcatctctcgcagcaccacgattgtgacagcgtatgtgatgactgtgacggggctaggctggcgggacgtgcttgaagccatcaaggccaccaggcccatcgccaaccccaacccaggctttaggcagcagcttaagagtttggctgggccagttcccagaaggatggtcaccggacccaaagtaccagatctgtctgtgcttcggtgaggaggacccgggccccacacagcaccccaaggagcagctcatcatggcggacctagtctctcttctttattctgggggctgggaaggatcccaaaacagggaacttggccgaaccctgggctttggatgctaaccactgaagtaccagcacctgtaggatgctgtctttgaagaaactgaggcggacctccaaatgcagccctaaggcagaggtcaacgtggaagaccagcccttctccaagccccactggtctttgcaagctgtacgttgtaggcaatctgagaactggaaagggggactacaaccagaaagttggttaccctgccatgggaataaagtagctgttttccaccccccaaaaaaaaaaaaaaaaaaaaaaaaaaa

SEQ ID NO: 10 - protein, 159 aa, Homo sapiens
MGNGMTKVLPGLYLGNFIDAKDLDQLGRNKITHIISIHESPQPLLQDITYLRIPVADTPEVPIKKHFKECINFIHCCRLNGGNCLVHCFAGISRSTTIVTAYVMTVTGLGWRDVLEAIKATRPIANPNPGFRQQLKSLAGPVPRRMVTGPKVPDLSVLR

SEQ ID NO: 11 - DNA, 1064 bp, Homo sapiens
ggccccccgttccccgccaggctgcaggcgtcgggcctgggccgtcagggcagctgtgaccggatcgcttcccgggcggcgagctgggggtgcacccggaccgccgcccccgggatcatgggcaatggcatgaccaaggtacttcctggactctacctcggaaacttcattgatgccaaagacctggatcagctgggccgaaataagatcacacacatcatctctatccatgagtcaccccagcctctgctgcaggatatcacctaccttcgcatcccggtcgctgatacccctgaggtacccatcaaaaagcacttcaaagaatgtatcaacttcatccactgctgccgccttaatggggggaactgccttgtgcactgctttgcaggcatctctcgcagcaccacgattgtgacagcgtatgtgatgactgtgacggggctaggctggcgggacgtgcttgaagccatcaaggccaccaggcccatcgccaaccccaacccaggctttaggcagcagcttgaagagtttggctgggccagttcccagaagggcttttaccaacctcataagctgttgtgagaaccaattgagacactgcaggaaagtgtttagccaggcccagcactgatgagcagtcggatggtcaccggacccaaagtaccagatctgtctgtgcttcggtgaggaggacccgggccccacacagcaccccaaggagcagctcatcatggcggacctagtctctcttctttattctgggggctgggaaggatcccaaaacagggaacttggccgaaccctgggctttggatgctaaccactgaagtaccagcacctgtaggatgctgtctttgaagaaactgaggcggacctccaaatgcagccctaaggcagaggtcaacgtggaagaccagcccttctccaagccccactggtctttgcaagctgtacgttgtaggcaatctgagaactggaaagggggactacaaccagaaagttggttaccctgccatgggaataaagtagctgttttccaaaaaaaaaaaaaaaaaaaaaaaaaa

SEQ ID NO: 12 - protein, 154 aa, Homo sapiens
MGNGMTKVLPGLYLGNFIDAKDLDQLGRNKITHIISIHESPQPLLQDITYLRIPVADTPEVPIKKHFKECINFIHCCRLNGGNCLVHCFAGISRSTTIVTAYVMTVTGLGWRDVLEAIKATRPIANPNPGFRQQLEEFGWASSQKGFYQPHKLL

SEQ ID NO: 13 - DNA, 833 bp, Homo sapiens
ggccccccgttccccgccaggctgcaggcgtcgggcctgggccgtcagggcagctgtgaccggatcgcttcccgggcggcgagctgggggtgcacccggaccgccgcccccgggatcatgggcaatggcatgaccaaggtacttcctggactctacctcggaaacttcattgatgccaaagacctggatcagctgggccgaaataagatcacacacatcatctctatccatgagtcaccccagcctctgctgcaggatatcacctaccttcgcatcccggtcgctgatacccctgaggtacccatcaaaaagcacttcaaagaatgtatcaacttcatccactgctgccgccttaatggggggaactgccttgtgcactgctttgcaggcatctctcgcagcaccacgattgtgacagcgtatgtgatgactgtgacggggctaggctggcgggacgtgcttgaagccatcaaggccaccaggcccatcgccaaccccaacccaggctttaggcagcagcttgaagagtttggctgggccagttcccagaagcttcgccggcagctggaggagcgcttcggcgagagccccttccgcgacgaggaggagttgcgcgcgctgctgccgctgtgcaagcgctgccggcagggctccgcgacctcggcctcctccgccgggccgcactcagcagcctccgagggaaccgtgcagcgcctggtgccgcgcacgccccgggaagcccaccggccgctgccgctgctggcgcgcgtcaagcagactttctcttgcctcccccggtgtctgtcccgcaagggcggcaagtgaggatgcag

SEQ ID NO: 14 - protein, 235 aa, Homo sapiens
MGNGMTKVLPGLYLGNFIDAKDLDQLGRNKITHIISIHESPQPLLQDITYLRIPVADTPEVPIKKHFKECINFIHCCRLNGGNCLVHCFAGISRSTTIVTAYVMTVTGLGWRDVLEAIKATRPIANPNPGFRQQLEEFGWASSQKLRRQLEERFGESPFRDEEELRALLPLCKRCRQGSATSASSAGPHSAASEGTVQRLVPRTPREAHRPLPLLARVKQTFSCLPRCLSRKGGK

SEQ ID NO: 15 - protein, 184 aa, Homo sapiens
MGNGMNKILPGLYIGNFKDARDAEQLSKNKVTHILSVHDSARPMLEGVKYLCIPAADSPSQNLTRHFKESIKFIHECRLRGESCLVHCLAGVSRSVTLVIAYIMTVTDFGWEDALHTVRAGRSCAMPMVGFQRQLQEFEKHEVHQYRQWLKEEYGESPLQDAEEAKNILAAPGILKFWAFLRRL

SEQ ID NO: 16 - protein, 24 aa, Homo sapiens
NGRVLVHCQAGISRSGTNILAYLM

SEQ ID NO: 17 - protein, 21 aa, Homo sapiens
CLVHCFAGISRSTTIVTAYVM

SEQ ID NO: 18 - DNA, 39 bp, Artificial Sequence
  Oligonucleotide primer used for PCR.
ggaattcaaggcaatggcatgaccaaggtacttcctgga

SEQ ID NO: 19 - DNA, 26 bp, Artificial Sequence
  Oligonucleotide primer used for PCR.
ggaattcacttgccgcccttgcggga

SEQ ID NO: 20 - DNA, 25 bp, Artificial Sequence
  Oligonucleotide primer used for PCR.
gcatcccggtcgctgatacccctga

SEQ ID NO: 21 - DNA, 24 bp, Artificial Sequence
  Oligonucleotide primer used for PCR.
gctaggctggcgggacgtgcttga

SEQ ID NO: 22 - DNA, 25 bp, Artificial Sequence
  Oligonucleotide primer used for PCR.
tcaggggtatcagcgaccgggatgc

SEQ ID NO: 23 - DNA, 24 bp, Artificial Sequence
  Oligonucleotide primer used for PCR.
tcaagcacgtcccgccagcctagc

SEQ ID NO: 24 - DNA, 29 bp, Artificial Sequence
  Oligonucleotide primer used for PCR.
caccccagcctctgctgcaggatatcacc

SEQ ID NO: 25 - DNA, 28 bp, Artificial Sequence
  Oligonucleotide primer used for PCR.
ctggcccagccaaactcttcaagctgtg

SEQ ID NO: 26 - DNA, 21 bp, Artificial Sequence
  Oligonucleotide primer used for PCR.
gcagcagcttgaagagtttgg

SEQ ID NO: 27 - DNA, 20 bp, Artificial Sequence
  Oligonucleotide primer used for PCR.
gggcaccagaggtttttgag

SEQ ID NO: 28 - DNA, 28 bp, Artificial Sequence
  Oligonucleotide primer used for PCR.
cctatgtctggcacccttctgggaactg

SEQ ID NO: 29 - DNA, 38 bp, Artificial Sequence
  Oligonucleotide primer used for PCR.
ggaactgccttgtgcactcctttgcaggcatctctcgc

SEQ ID NO: 30 - DNA, 38 bp, Artificial Sequence
  Oligonucleotide primer used for PCR.
gcgagagatgcctgcaaaggagtgcacaaggcagttcc

SEQ ID NO: 31 - protein, 329 aa, Homo sapiens [sequence omitted]

SEQ ID NO: 32 - protein, 235 aa, Homo sapiens [sequence omitted]

SEQ ID NO: 33 - protein, 289 aa, Homo sapiens
AVAVAGQAWAGPRTPGPPFPARLQASGLGRQGSCDRIASRAASWGCTRTAAPGIMGNGMTKVLPGLYLGNFIDAKDLDQLGRNKITHIISIHESPQPLLQDITYLRIPVADTPEVPIKKHFKECINFIHCCRLNGGNCLVHCFAGISRSTTIVTAYVMTVTGLGWRDVLEAIKATRPIANPNPGFRQQLEEFGWASSQKLRRQLEERFGESPFRDEEELRALLPLCKRCRQGSATSASSAGPHSAASEGTLQRLVPRTPREAHRPLPLLARVKQTFSCLPRCLSRKGGK

SEQ ID NO: 34 - DNA, 926 bp, Homo sapiens
ccccgccgctcctcctccctgtaacatgccatagtgcgcctgcgaccacacggccggggcgctagcgttcgccttcagccaccatggggaatgggatgaacaagatcctgcccggcctgtacatcggcaacttcaaagatgccagagacgcggaacaattgagcaagaacaaggtgacacatattctgtctgtccacgatagtgccaggcctatgttggagggagttaaatacctgtgcatcccagcagcggattcaccatctcaaaacctgacaagacatttcaaagaaagtattaaattcattcacgagtgccggctccgcggtgagagctgccttgtacactgcctggccggggtctccaggagcgtgacactggtgatcgcatacatcatgaccgtcactgactttggctgggaggatgccctgcacaccgtgcgtgctgggagatcctgtgccaaccccaacgtgggcttccagagacagctccaggagtttgagaagcatgaggtccatcagtatcggcagtggctgaaggaagaatatggagagagccctttgcaggatgcagaagaagccaaaaacattctggccgctccaggaattctgaagttctgggcctttctcagaagactgtaatgtacctgaagtttctgaaatattgcaaacccgcagagtttaggctggtgctgccaaaaagaaaagcaacatagagtttaagtatccagtagtgatttgtaaacttgtttttcatttgaagctgaatatatacgtagtcatgtttatgttgagaactaaggatattctttagcaagagaaaatattttccccttatccccactgctgtggaggtttctgtacctcgcttggatgcctgtaaggatcccgggagccttgccgcactgccttgtgggtggcttggcgctc

SEQ ID NO: 35 - protein, 6 aa, Artificial Sequence
  Preferred substrate for PTB1B, corresponding to residues 988-993 of human EGF receptor.
DADEYL

SEQ ID NO: 36 - protein, 11 aa, Artificial Sequence
  DSP-18 peptide 18-1
IDAKDLDQLGR

SEQ ID NO: 37 - protein, 11 aa, Artificial Sequence
  DSP-18 peptide 18-2
VADTPEVPIKK

SEQ ID NO: 38 - protein, 149 aa, Unknown
  Consensus sequence based on multiple phosphatase sequence alignments.
MGNGMTKVLPGLYLGNFIDAKDLDQLGRNKITHIISIHESPQPLLQDITYLRIPVADTPEVPIKKHFKECINFIHCCRLNGGNCLVHCFAGISRSTTIVTAYVMTVTGLGWRDVLEAIKATRPIANPNPGFRQQLEEFGWASSQKGRGP

SEQ ID NO: 39 - DNA, 1326 bp, Homo sapiens
atgcaggggcagactgtagttccaaaagattcctacactatatcccttatccagaggctgcggggccgtgaggccgcaaggagaacccatgagaaccttcttcggctgtctgccctagtgagatccccacagacagctagcatcgactgccacacgtggtcagtttctagtggaaccaatacttcgctgcaggcgtcgggcctgggccgtcagggcagctgtgaccggatcgcttcccgggcggcgagctgggggtgcacccggaccgccgcccccgggatcatgggcaatggcatgaccaaggtacttcctggactctacctcggaaacttcattgatgccaaagacctggatcagctgggccgaaataagatcacacacatcatctctatccatgagtcaccccagcctctgctgcaggatatcacctaccttcgcatcccggtcgctgatacccctgaggtacccatcaaaaagcacttcaaagaatgtatcaacttcatccactgctgccgccttaatggggggaactgccttgtgcactgctttgcaggcatctctcgcagcaccacgattgtgacagcgtatgtgatgactgtgacggggctaggctggcgggacgtgcttgaagccatcaaggccaccaggcccatcgccaaccccaacccaggctttaggcagcagcttgaagagtttggctgggccagttcccagaagcttcgccggcagctggaggagcgcttcggcgagagccccttccgcgacgaggaggagttgcgcgcgctgctgccgctgtgcaagcgctgccggcagggctccgcgacctcggcctcctccgccgggccgcactcagcagcctccgagggaaccgtgcagcgcctggtgccgcgcacgccccgggaagcccaccggccgctgccgctgctggcgcgcgtcaagcagactttctcttgcctcccccggtgtctgtcccgcaagggcggcaagtgaggatgcagtccagccgtggctccccacttccgactggctcccttcgggggctgtctgcgccttccacgccccccaggacgggcccagaggctgggggagccccgcggcggcctgaaccctgcctcccgcgcccgccctgctcgtccgcgtctgcagtcagcgtccccaacctgtgcgtctctgtgtccgggccggcctgctgcagccacctggtgccttagtccttgggctgggggagggggcccacccttaaaggcggcgggaggggagggagggagagtggagggtttgacgggcctggagggtattaaagagacacagaagaagctgcctgtc

SEQ ID NO: 40 - DNA, 705 bp, Homo sapiens
atgggcaatggcatgaccaaggtacttcctggactctacctcggaaacttcattgatgccaaagacctggatcagctgggccgaaataagatcacacacatcatctctatccatgagtcaccccagcctctgctgcaggatatcacctaccttcgcatcccagtcgctgatacccctgaggtacccatcaaaaagcacttcaaagaatgtatcaacttcatccactgctgccgccttaatggggggaactgccttgtgcactgctttgcaggcatctctcgcagcaccacgattgtgacagcgtatgtgatgactgtgacggggctaggctggcgggacgtgcttgaagccatcaaggccaccaggcccatcgccaaccccaacccaggctttaggcagcagcttgaagagtttggctgggccagttcccagaagcttcgccggcagctggaggagcgcttcggcgagagccccttccgcgacgaggaggagttgcgcgcgctgctgccgctgtgcaagcgctgccggcagggctccgcgacctcggcctcctccgccgggccgcactcagcagcctccgagggaaccctgcagcgcctggtgccgcgcacgccccgggaagcccaccggccgctgccgctgctggcgcgcgtcaagcagactttctcttgcctcccccggtgtctgtcccgcaagggcggcaag

SEQ ID NO: 41 - DNA, 867 bp, Homo sapiens
gcggtggcggtggctgggcaggcctgggcagggccgcggacgccaggccccccgttccccgccaggctgcaggcgtcgggcctgggccgtcagggcagctgtgaccggatcgcttcccgggcggcgagctgggggtgcacccggaccgccgcccccgggatcatgggcaatggcatgaccaaggtacttcctggactctacctcggaaacttcattgatgccaaagacctggatcagctgggccgaaataagatcacacacatcatctctatccatgagtcaccccagcctctgctgcaggatatcacctaccttcgcatcccagtcgctgatacccctgaggtacccatcaaaaagcacttcaaagaatgtatcaacttcatccactgctgccgccttaatggggggaactgccttgtgcactgctttgcaggcatctctcgcagcaccacgattgtgacagcgtatgtgatgactgtgacggggctaggctggcgggacgtgcttgaagccatcaaggccaccaggcccatcgccaaccccaacccaggctttaggcagcagcttgaagagtttggctgggccagttcccagaagcttcgccggcagctggaggagcgcttcggcgagagccccttccgcgacgaggaggagttgcgcgcgctgctgccgctgtgcaagcgctgccggcagggctccgcgacctcggcctcctccgccgggccgcactcagcagcctccgagggaaccctgcagcgcctggtgccgcgcacgccccgggaagcccaccggccgctgccgctgctggcgcgcgtcaagcagactttctcttgcctcccccggtgtctgtcccgcaagggcggcaag

SEQ ID NO: 42 - DNA, 1160 bp, Homo sapiens
gggcggtggcggtggctgggcaggcctgggcagggccgcggacgccaggccccccgttccccgccaggctgcaggcgtcgggcctgggccgtcagggcagctgtgaccggatcgcttcccgggcggcgagctgggggtgcacccggaccgccgcccccgggatcatgggcaatggcatgaccaaggtacttcctggactctacctcggaaacttcattgatgccaaagacctggatcagctgggccgaaataagatcacacacatcatctctatccatgagtcaccccagcctctgctgcaggatatcacctaccttcgcatcccagtcgctgatacccctgaggtacccatcaaaaagcacttcaaagaatgtatcaacttcatccactgctgccgccttaatggggggaactgccttgtgcactgctttgcaggcatctctcgcagcaccacgattgtgacagcgtatgtgatgactgtgacggggctaggctggcgggacgtgcttgaagccatcaaggccaccaggcccatcgccaaccccaacccaggctttaggcagcagcttgaagagtttggctgggccagttcccagaagcttcgccggcagctggaggagcgcttcggcgagagccccttccgcgacgaggaggagttgcgcgcgctgctgccgctgtgcaagcgctgccggcagggctccgcgacctcggcctcctccgccgggccgcactcagcagcctccgagggaaccctgcagcgcctggtgccgcgcacgccccgggaagcccaccggccgctgccgctgctggcgcgcgtcaagcagactttctcttgcctcccccggtgtctgtcccgcaagggcggcaagtgaggatgcagtccagccgtggctccctacttccgactggctcccttcgggggctgtctgcgccttccacgccctgctcgtccgcgtctgcagtcagcgtccccaacctgtgcgtctctgtgtccgggccggcctgctgcagccacctggtgccttagtccttgggctgggggagggggcccacccttaaaggcggcgggaggggagggagggagagtggagggtttgacgggcctggagggtattaaagagacacagaagaaaaaaaaaaaaaaaaagggcggccgctag

(19) United States
(12) Patent Application Publication
(10) Pub. No.: US 2003/0092114 A1
(43) Pub. Date: May 15, 2003

(54) DSP-18 dual-specificity phosphatase

(75) Inventors: Ralf M. Luche, Seattle, WA (US); Bo Wei, Kirkland, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: CEPTYR, Inc., Bothell, WA (US)

(21) Appl. No.: 10/151,320

(22) Filed: May 16, 2002

Related U.S. Application Data
(60) Provisional application No. 60/291,476, filed on May 16, 2001.

(51) Int. Cl. C12P 21/02; C12N 5/06; C07H 21/04; C12N 9/16

(52) U.S. Cl. 435/69.1; 435/196; 435/320.1; 435/325; 536/23.2

(57) ABSTRACT

Compositions and methods are provided for the treatment of conditions associated with cell proliferation, cell differentiation and/or cell survival. In particular, the dual-specificity phosphatase DSP-18 isoforms DSP-18a-f, and polypeptide variants thereof that stimulate dephosphorylation of DSP-18 substrates, are provided. The polypeptides may be used, for example, to identify antibodies and other agents that inhibit DSP-18 activity. The polypeptides and agents may be used to modulate cell proliferation, cell differentiation and cell survival.

### CROSS-REFERENCE TO RELATED APPLICATION

This application claims the benefit of U.S. Provisional Patent Application No. 60/291,476 filed May 16, 2001, which is incorporated herein by reference in its entirety.

### TECHNICAL FIELD

The present invention relates generally to compositions and methods useful for treating conditions associated with defects in cell proliferation, cell differentiation and/or cell survival. The invention is more particularly related to dual-specificity protein phosphatases, and polypeptide variants thereof. The present invention is also related to the use of such polypeptides to identify antibodies and other agents, including small molecules, that modulate signal transduction leading to proliferative responses, cell differentiation and/or cell survival.

### BACKGROUND OF THE INVENTION

Mitogen-activated protein kinases (MAP-kinases) are present as components of conserved cellular signal transduction pathways that have a variety of conserved members. MAP-kinases are activated by phosphorylation at a dual phosphorylation motif with the sequence Thr-X-Tyr (by MAP-kinase kinases), in which phosphorylation at the tyrosine and threonine residues is required for activity. Activated MAP-kinases phosphorylate several transduction targets, including transcription factors. Inactivation of MAP-kinases is mediated by dephosphorylation at this site by dual-specificity phosphatases referred to as MAP-kinase phosphatases. In higher eukaryotes, the physiological role of MAP-kinase signaling has been correlated with cellular events such as proliferation, oncogenesis, development and differentiation. Accordingly, the ability to regulate signal transduction via these pathways could lead to the development of treatments and preventive therapies for human diseases associated with MAP-kinase signaling, such as cancer.

Dual-specificity protein tyrosine phosphatases (dual-specificity phosphatases) are phosphatases that dephosphorylate both phosphotyrosine and phosphothreonine/serine residues (Walton et al., Ann. Rev. Biochem. 62:101-120, 1993). Several dual-specificity phosphatases that inactivate a MAP-kinase have been identified, including MKP-1 (WO 97/00315; Keyse and Emslie, Nature 59:644-647, 1992), MKP-4, MKP-5, MKP-7, Hb5 (WO 97/06245), PAC1 (Ward et al., Nature 367:651-654, 1994), HVH2 (Guan and Butch, J. Biol. Chem. 270:7197-7203, 1995) and PYST1 (Groom et al., EMBO J 15:3621-3632, 1996). Expression of certain dual-specificity phosphatases is induced by stress or mitogens, but others appear to be expressed constitutively in specific cell types. The regulation of dual-specificity phosphatase expression and activity is critical for control of MAP-kinase mediated cellular functions, including cell proliferation, cell differentiation, and cell survival. For example, dual-specificity phosphatases may function as negative regulators of cell proliferation. It is likely that there are many such dual-specificity phosphatases, each with varying specificity with regard to cell type or activation. However, the regulation of dual specificity phosphatases remains poorly understood, and only a relatively small number of dual-specificity phosphatases have been identified.

Accordingly, a need exists in the art for an improved understanding of MAP-kinase signaling, and the regulation of dual-specificity phosphatases within MAP-kinase signaling cascades. An increased understanding of dual-specificity phosphatase regulation may facilitate the development of methods for modulating the activity of proteins involved in MAP-kinase cascades, and for treating conditions associated with such cascades. The present invention fulfills these needs and further provides other related advantages.

### SUMMARY OF THE INVENTION

Briefly stated, the present invention provides compositions and methods for identifying agents capable of modulating cellular proliferative responses. In one aspect, the present invention provides an isolated DSP-18 polypeptide comprising a DSP-18a amino acid sequence of DSP-18a as set forth in SEQ ID NO:2, or a variant thereof that differs in one or more amino acid deletions, additions, insertions, or substitutions at no more than 15% of the amino acids in SEQ ID NO:2 such that the polypeptide retains the ability to dephosphorylate an activated MAP-kinase; an isolated DSP-18 polypeptide comprising a DSP-18b amino acid sequence as set forth in SEQ ID NO:4, or a variant thereof that differs in one or more amino acid deletions, additions, insertions, or substitutions at no more than 25% of the amino acids in SEQ ID NO:4 such that the polypeptide retains the ability to dephosphorylate an activated MAP-kinase; an isolated DSP-18 polypeptide comprising a DSP-18c amino acid sequence as set forth in SEQ ID NO:6, or a variant thereof that differs in one or more amino acid deletions, additions, insertions, or substitutions at no more than 15% of the amino acids in SEQ ID NO:6 such that the polypeptide retains the ability to dephosphorylate an activated MAP-kinase; an isolated DSP-18 polypeptide comprising a DSP-18d amino acid sequence as set forth in SEQ ID NO:8, or a variant thereof that differs in one or more amino acid deletions, additions, insertions, or substitutions at no more than 15% of the amino acids in SEQ ID NO:8 such that the polypeptide retains the ability to dephosphorylate an activated MAP-kinase; an isolated DSP-18 polypeptide comprising a DSP-18e amino acid sequence as set forth in SEQ ID NO:10, or a variant thereof that differs in one or more amino acid deletions, additions, insertions, or substitutions at no more than 12% of the amino acids in SEQ ID NO:10 such that the polypeptide retains the ability to dephosphorylate an activated MAP-kinase; an isolated DSP-18 polypeptide comprising a DSP-18f amino acid sequence as set forth in SEQ ID NO:12, or a variant thereof that differs in one or more amino acid deletions, additions, insertions, or substitutions at no more than 5% of the amino acids in SEQ ID NO:12 such that the polypeptide retains the ability to dephosphorylate an activated MAP-kinase; an isolated prototypical DSP-18 polypeptide consisting of a DSP-18pr amino acid sequence as set forth in SEQ ID NO:14 such that the polypeptide retains the ability to dephosphorylate an activated MAP-kinase.

In another embodiment, the present invention provides an isolated polynucleotide that encodes at least 147 consecutive amino acids of a polypeptide having a sequence corresponding to any one of the sequences selected from the group consisting of SEQ ID NOS:2, 4, 6, 8, and 12. In a certain embodiment, the invention provides an isolated polynucleotide that encodes at least 136 consecutive amino acids of a polypeptide having an amino acid sequence corresponding to the sequence set forth in SEQ ID NO: 10. Certain such polynucleotides encode a DSP-18 polypeptide such as a prototypical DSP-18 and certain preferred such polynucleotides encode a DSP-18a, a DSP-18b, a DSP-18c, a DSP-18d, a DSP-18e or a DSP-18f polypeptide. Still further, polynucleotides may be antisense polynucleotides that comprise at least 15 consecutive nucleotides complementary to a portion of a DSP-18 polynucleotide. In certain embodiments, polynucleotides may detectably hybridize under conditions that include a wash in 0.1×SSC and 0.1% SDS at 50° C. for 15 minutes to a polynucleotide having a sequence that is complementary to positions 553-660 as set forth in SEQ ID NO:1; a polynucleotide having a sequence that is complementary to positions 553-1011 as set forth in SEQ ID NO:3; a polynucleotide having a sequence that is complementary to positions 553-660 as set forth in SEQ ID NO:5; a polynucleotide having a sequence that is complementary to positions 553-660 as set forth in SEQ ID NO:7; a polynucleotide having a sequence that is complementary to positions 523-594 as set forth in SEQ ID NO:9; and a polynucleotide having a sequence that is complementary to positions 553-579 as set forth in SEQ ID NO:11. Also provided are expression vectors comprising any of the foregoing polynucleotides, and host cells transformed or transfected with such expression vectors.

The present invention further provides methods for producing a DSP-18 polypeptide (such as a DSP-18pr, DSP-18a, a DSP-18b, a DSP-18c, a DSP-18d, a DSP-18e or a DSP-18f polypeptide), comprising the steps of: (a) culturing a host cell as described above under conditions that permit expression of the DSP-18 polypeptide; and (b) isolating DSP-18 polypeptide from the host cell culture.

Also provided by the present invention are isolated antibodies, and antigen binding fragments thereof, that specifically bind to a DSP-18 polypeptide, such as a polypeptide having the DSP-18 sequence of any one of SEQ ID NOS:2, 4, 6, 8, 10, or 12 . wherein the antibody or antigen binding fragment thereof does not specifically bind to a SPG008 polypeptide having the sequence set forth in SEQ ID NO:31 or to a 69109 polypeptide having the amino acid sequence set forth in SEQ ID NO:32 or SEQ ID NO:33. The invention further provides an isolated antibody that specifically binds to an antigenic determinant of a DSP-18 polypeptide having an amino acid sequence selected from the group consisting of the sequence set forth in any one of SEQ ID NO:2, SEQ ID NO:6, and SEQ ID NO:8, wherein the antigenic determinant comprises at least one amino acid located at positions 146-181 of SEQ ID NO:2, SEQ ID NO:6, or SEQ ID NO:8. The invention also provides an isolated antibody that specifically binds to an antigenic determinant of a DSP-18 polypeptide having an amino acid sequence as set forth in SEQ ID NO:4, wherein the antigenic determinant comprises at least one amino acid located at positions 146-298 in SEQ ID NO:4; an isolated antibody that specifically binds to an antigenic determinant of a DSP-18 polypeptide having an amino acid sequence as set forth in SEQ ID NO:10, wherein the antigenic determinant comprises at least one amino acid located at positions 136-159 of SEQ ID NO:10; an isolated antibody that specifically binds to an antigenic determinant of a DSP-18 polypeptide having an amino acid sequence as set forth in SEQ ID NO:12, wherein the antigenic determinant comprises at least one amino acid located at positions 146-154 of SEQ ID NO:12. Within further embodiments, the present invention provides an isolated antibody that specifically binds to an antigenic determinant of a DSP-18 polypeptide having an amino acid sequence selected from the group consisting of SEQ ID NOS:2, 4, 6, 8, 10, 12, and 14, wherein the antigenic determinant comprises at least one amino acid located at positions 18-28 of any one of SEQ ID NOS: 2, 4, 6, 8, 10, 12, and 14, and an isolated antibody that specifically binds to an antigenic determinant of a DSP-18 polypeptide having an amino acid sequence selected from the group consisting of SEQ ID NOS:2, 4, 6, 8, 10, 12, and 14, wherein the antigenic determinant comprises at least one amino acid located at positions 55-65 of any one of SEQ ID NOs: 2, 4, 6, 8, 10, 12, and 14.

The present invention further provides, within other embodiments, pharmaceutical compositions comprising a polypeptide, polynucleotide, antibody or fragment thereof as described above in combination with a physiologically acceptable carrier.

Within further embodiments, the present invention provides methods for detecting the presence of a DSP-18 polypeptide in a sample, comprising: (a) contacting a sample with an antibody or an antigen-binding fragment thereof as described above, under conditions and for a time sufficient to allow formation of an antibody/DSP-18 polypeptide complex; and (b) detecting a level of antibody/DSP-18 complex. The antibody contacting the sample may be linked to a support material or to a detectable marker.

Within still other embodiments, the present invention provides methods for detecting DSP-18 expression in a sample, comprising: (a) contacting a sample with an antisense polynucleotide or an isolated DSP-18 polynucleotide as described above; and (b) detecting presence in the sample of a polynucleotide that hybridizes to the antisense polynucleotide or to an isolated DSP-18 polynucleotide. The amount of DSP-18 polynucleotide that hybridizes to the antisense polynucleotide or to an isolated DSP-18 polynucleotide may be determined, for example, using polymerase chain reaction or a hybridization assay.

The invention also provides DSP-18 polypeptides useful in screening assays for modulators of enzyme activity and/or substrate binding. Methods are also provided, within other embodiments, for screening for an agent that modulates DSP-18 activity, comprising the steps of: (a) contacting a candidate agent with a DSP-18 polypeptide as described above, under conditions and for a time sufficient to permit interaction between the polypeptide and candidate agent; and (b) subsequently evaluating the ability of the polypeptide to dephosphorylate a DSP-18 substrate, relative to the ability of the polypeptide to dephosphorylate the DSP-18 substrate in the absence of candidate agent. Such methods may be performed in vitro or in a cellular environment (e.g. within an intact cell).

In other embodiments of the invention, methods are provided for screening for an agent that modulates DSP-18, comprising the steps of: (a) contacting a candidate agent with a cell comprising a DSP-18 promoter operably linked to a polynucleotide encoding a detectable transcript or protein, under conditions and for a time sufficient to permit interaction between the promoter and candidate agent; and (b) subsequently evaluating the expression of the polynucleotide, relative to a level of expression in the absence of candidate agent.

Also provided are methods for modulating a proliferative response in a cell, comprising contacting a cell with an agent that modulates DSP-18 activity. Within other embodiments, methods are provided for modulating differentiation of a cell, comprising contacting a cell with an agent that modulates DSP-18 activity. The present invention further provides methods for modulating cell survival, comprising contacting a cell with an agent that modulates DSP-18 activity.

Within related embodiments, the present invention provides methods for treating a patient afflicted with a disorder associated with DSP-18 activity (or treatable by administration of DSP-18), comprising administering to a patient a therapeutically effective amount of an agent that modulates DSP-18 activity. Such disorders include Duchenne muscular dystrophy, cancer, graft-versus-host disease, autoimmune diseases, allergies, metabolic diseases, abnormal cell growth, abnormal cell proliferation and cell cycle abnormalities.

Within further embodiments, DSP-18 substrate trapping mutant polypeptides are provided. Such polypeptides differ from a DSP-18a amino acid sequence set forth in SEQ ID NO:2, or a variant thereof in one or more amino acid deletions, additions, insertions, or substitutions at no more than 15% of the amino acids in SEQ ID NO:2; a DSP-18b amino acid sequence set forth in SEQ ID NO:4, or a variant thereof in one or more amino acid deletions, additions, insertions, or substitutions at no more than 25% of the amino acids in SEQ ID NO:4; from aDSP-18c amino acid sequence set forth in SEQ ID NO:6, or a variant thereof in one or more amino acid deletions, additions, insertions, or substitutions at no more than 15% of the amino acids in SEQ ID NO:6; a DSP-18d amino acid sequence set forth in SEQ ID NO:8, or a variant thereof in one or more amino acid deletions, additions, insertions, or substitutions at no more than 15% of the amino acids in SEQ ID NO:8, a DSP-18e amino acid sequence set forth in SEQ ID NO:10, or a variant thereof in one or more amino acid deletions, additions, insertions, or substitutions at no more than 12% of the amino acids in SEQ ID NO:10, a DSP-18f amino acid sequence set forth in SEQ ID NO:12, or a variant thereof in one or more amino acid deletions, additions, insertions, or substitutions at no more than 5% of the amino acids in SEQ ID NO:12, such that the DSP-18a-f substrate trapping mutant polypeptides bind to a substrate with an affinity that is not substantially diminished relative to DSP-18a, DSP-18b, DSP-18c, DSP18d, DSP-18e, DSP-18f, respectively. Still further, the invention provides a DSP-18pr substrate trapping mutant polypeptide that differs from an amino acid sequence set forth in SEQ ID NO:14, or a variant thereof in one or more amino acid deletions, additions, insertions, or substitutions at no more than 20% of the amino acids in SEQ ID NO:14, and such that the polypeptide binds to a substrate with an affinity that is not substantially diminished relative to DSP-18pr, and such that the ability of the polypeptide to dephosphorylate a substrate is reduced relative to DSP-18pr, wherein the DSP-18pr substrate trapping mutant polypeptide does not consist of an amino acid sequence set forth in SEQ ID NOS:31-33. Within certain specific embodiments, a DSP-18 substrate trapping mutant polypeptide contains a substitution at position 72 or position 103 of the DSP-18 polypeptide having the amino acid sequence set forth in any one of SEQ ID NOS:2, 4, 6, 8, 10, 12 or 14.

The present invention further provides, within other embodiments, methods for screening a molecule for the ability to interact with DSP-18, comprising the steps of: (a) contacting a candidate molecule with an isolated DSP-18 polypeptide as described above under conditions and for a time sufficient to permit the candidate molecule and the DSP-18 polypeptide to interact; and (b) detecting the presence or absence of binding of the candidate molecule to the DSP-18 polypeptide. The step of detecting may comprise, for example, an affinity purification step, a yeast two hybrid screen or a screen of a phage display library.

In certain other embodiments, the invention provides an immunogen comprising a DSP-18 peptide comprising an amino acid sequence of at least ten consecutive amino acids of a polypeptide selected from the group consisting of DSP-18a as set forth in SEQ ID NO: 2, DSP-18b as set forth in SEQ ID NO:4, DSP-18c polypeptide as set forth in SEQ ID NO:6, DSP-18d polypeptide as set forth SEQ ID NO:8, DSP-18e polypeptide as set forth in SEQ ID NO:10, DSP-18f polypeptide as set forth in SEQ ID NO:12, and DSP-18pr polypeptide as set forth in SEQ ID NO:14. Within certain specific embodiments, an immunogen comprises an amino acid sequence as set forth in SEQ ID NO:36 and SEQ ID NO:37. The present invention also provides an immunogen comprising a DSP-18 peptide comprising an amino acid sequence of at least four consecutive amino acids selected from the group consisting of amino acids at positions 146-181 of a DSP-18a polypeptide as set forth in SEQ ID NO:2, amino acids at positions 146-298 of SEQ ID NO:4, amino acids at positions 146-181 of a DSP-18c polypeptide as set forth in SEQ ID NO:6, amino acids at positions 146-181 of a DSP-18d polypeptide as set forth in SEQ ID NO:8, amino acids at positions 136-159 of a DSP-18e polypeptide as set forth in SEQ ID NO:10, and amino acids at positions 146-154 of DSP-18f as set forth in SEQ ID NO:12. In certain other embodiments, an immunogen comprises a DSP-18 peptide comprising an amino acid sequence of at least 11 consecutive amino acids at positions 136-181 of a DSP-18a, DSP-18c, or DSP-18d polypeptide as set forth in SEQ ID NOS:2, 6. or 8, or at amino acids at positions 136-298 of a DSP-18b polypeptide as set forth in SEQ ID NO:4.

These and other embodiments of the present invention will become apparent upon reference to the following detailed description and attached drawings. All references disclosed herein are hereby incorporated by reference in their entireties as if each was incorporated individually.

### BRIEF DESCRIPTION OF THE DRAWINGS

FIG. 1 presents an extended consensus cDNA sequence encoding prototypical DSP-18 (DSP-18pr) (FIG. 1A) [SEQ ID NO:13] and the deduced DSP-18pr amino acid sequence (FIG. 1B) [SEQ ID NO:14]. In FIG. 1A, initiating methionine (ATG) and stop (TGA) codons and intron/exon splice junctions are depicted in bold type with the splice donor sequences in bold without underscore, and the splice acceptor sequences in bold with underscore. In FIG. 1B, initiating methionine and the phosphatase active site are depicted in bold type.

FIG. 2 shows a schematic depiction of exon utilization in transcripts encoding DSP-18 polypeptides. Differential utilization of exons I-XI is depicted for DSP-18 isoforms described in the Examples; lightly and cross-hatched boxes and incompletely shaded boxes represent exons not utilized in all DSP-18 isoforms and partially utilized exons, respectively.

FIG. 3 presents nucleotide and amino acid sequences for a DSP-18 isoform, DSP-18a. FIG. 3A presents a cDNA sequence for DSP-18a [SEQ ID NO:1], with the start (ATG) and stop (TGA) codons and intron/exon splice junctions indicated in bold; intron/exon splice junctions are depicted in bold type with the splice donor sequences in bold without underscore and the splice acceptor sequences in bold with underscore. FIG. 3B presents the amino acid sequence of the DSP-18a polypeptide [SEQ ID NO:2] encoded by SEQ ID NO:1, with the phosphatase active site depicted in bold type.

FIG. 4 presents nucleotide and amino acid sequences for a DSP-18 isoform, DSP-18b. FIG. 4A presents a cDNA sequence for DSP-18b [SEQ ID NO:3], with the start (ATG) and stop (TGA) codons and intron/exon splice junctions indicated in bold; intron/exon splice junctions are depicted in bold type with the splice donor sequences in bold without underscore and the splice acceptor sequences in bold with underscore. FIG. 4B presents the amino acid sequence of the DSP-18b polypeptide [SEQ ID NO:4] encoded by SEQ ID NO:3, with the phosphatase active site depicted in bold type.

FIG. 5 presents nucleotide sequences for DSP-18 isoforms, DSP-18c and DSP-18d. FIG. 5A presents a cDNA sequence for DSP-18c [SEQ ID NO:5], with the start (ATG) and stop (TGA) codons and intron/exon splice junctions indicated in bold. FIG. 5B presents a cDNA sequence for DSP-18d [SEQ ID NO:7], with the start (ATG) and stop (TGA) codons and intron/exon splice junctions indicated in bold. DSP-18c [SEQ ID NO:6] encoded by SEQ ID NO:5, and DSP-18d [SEQ ID NO:8] encoded by SEQ ID NO:7, both share the 181 amino acid sequence encoded by the open reading frame of DSP-18a (see FIG. 3).

FIG. 6 presents nucleotide and amino acid sequences for DSP-18 isoforms, DSP-18e and DSP-18f. FIG. 6A presents a cDNA sequence for DSP-18e [SEQ ID NO:9], with the start (ATG) and stop (TGA) codons and intron/exon splice junctions indicated in bold. FIG. 6B presents the amino acid sequence of DSP-18e polypeptide [SEQ ID NO:10] encoded by SEQ ID NO:9, with the phosphatase active site sequence in boldface type. FIG. 6C-D presents nucleotide and amino acid sequences for DSP-18f. FIG. 6C presents a cDNA sequence for DSP-18f [SEQ ID NO:11], with the start (ATG) and stop (TGA) codons and intron/exon splice junctions indicated in bold. FIG. 6D presents the amino acid sequence of DSP-18f polypeptide [SEQ ID NO:12] encoded by SEQ ID NO:11, with the phosphatase active site sequence in boldface type.

FIG. 7 shows an alignment of the amino acid sequences of DSP-18a (SEQ ID NO:2, DSP-18b [SEQ ID NO:4], DSP-18e [SEQ ID NO:10], DSP-18f [SEQ ID NO:12], prototypical DSP-18 (DSP-18pr) [SEQ ID NO:14], with amino acid sequences as disclosed in International Application No. PCT/US00/34736 (SGP008, therein SEQ ID NO:20) [SEQ ID NO:31 herein] and in International Application No. PCT/US01/30118 (69109 polypeptides, therein SEQ ID NOs: 2 and 12) [SEQ ID NOs: 32 and 33 herein] and herein incorporated by reference.

FIG. 8 shows an alignment of the amino acid sequence of DSP-18a [SEQ ID NO:2] with the amino acid sequence of DSP-3 [SEQ ID NO:15] as disclosed in U.S. application Ser No. 09/608,062 and herein incorporated by reference.

FIG. 9 shows a northern blot analysis of DSP-18 expression in various cell and tissue types: Br, brain; He, heart; SkM, skeletal muscle; Co, colon; Th, thymus; Sp, spleen; Ki, kidney; Li, liver; SI, small intestine; P1, placenta; Lu, lung; pbl, peripheral blood lymphocytes.

FIG. 10 illustrates phosphatase activity of FLAG®-DSP-18pr (DSP18 WT), DSP-18pr D72A (DSP18 DA), and FLAG®-DSP-18pr C103S (DSP18 CS) using DifluoroMUP (DiFMUP) as a substrate.

FIG. 11 depicts immunoblot analysis of FLAG®-DSP-18pr, FLAG®-DSP-18pr D72A and FLAG®-DSP-18pr C103S substrate trapping mutant immunoprecipitated from 293-HEK cells that were transfected with recombinant FLAG®-DSP-18pr and FLAG®-DSP-18pr C103S expression constructs. Lane 1, 293-HEK cells transfected with empty vector; lane 2, lysate from untransfected 293-HEK cells; lane 3, FLAG®-DSP-18pr wildtype (WT); lane 4, FLAG®-DSP-18pr D72A; lane 5, FLAG®-DSP-18pr C103S.

FIG. 12 illustrates phosphatase activity of DSP-18pr and DSP-3 using DiFMUP as substrate (FIG. 12A) and ³²P-ERP (FIG. 12B). Duplicate samples of each dual specificity phosphatase are presented.

FIG. 13 shows an immunoblot analysis of isolated DSP-18 detected by affinity-purified rabbit IgG from rabbits immunized with DSP-18 peptide 18-1, IDAKDLDQLGR (SEQ ID NO:36) (lanes 1-3) or with DSP-18 peptide 18-2, VADTPEVPIKK (SEQ ID NO:37) (lanes 4-6). DSP-18pr was blotted against pre-immune sera from rabbits immunized with DSP-18-1 (Lane 1); purified rabbit anti-DSP-18-1 from Bleed 1 (2 mg/ml) (Lane 2); purified rabbit anti-DSP-18-1 from Bleed 2 (2 mg/ml) (Lane 3); pre-immune sera from rabbits immunized with DSP-18-2 (Lane 4); purified rabbit anti-DSP-18-2 from Bleed 1 (2 mg/ml) (Lane 5); and purified rabbit anti-DSP-18-2 from Bleed 2 (2 mg/ml) (Lane 6).

### DETAILED DESCRIPTION OF THE INVENTION

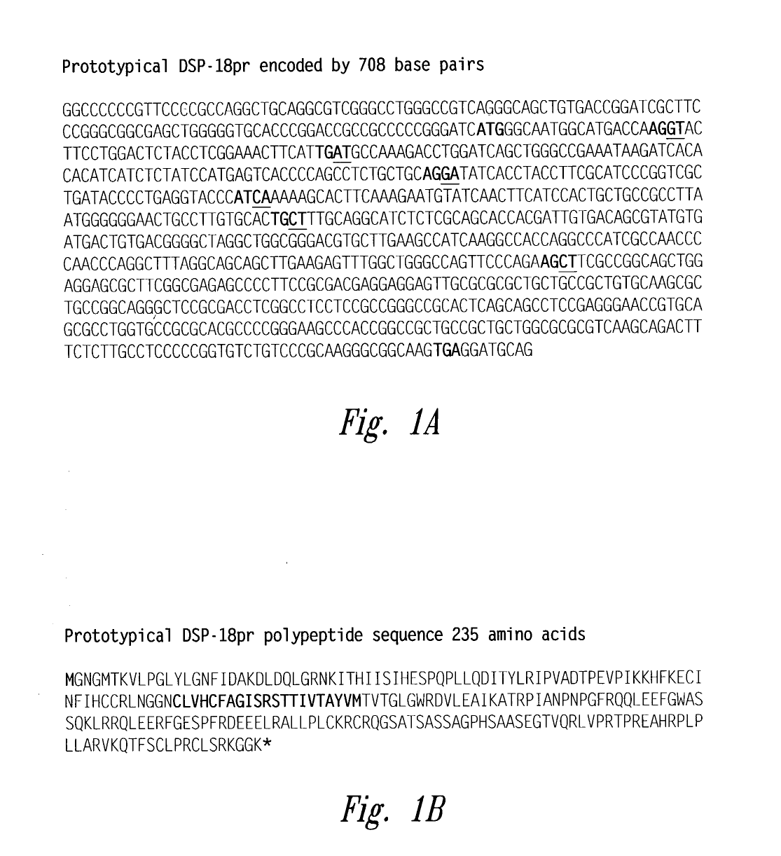

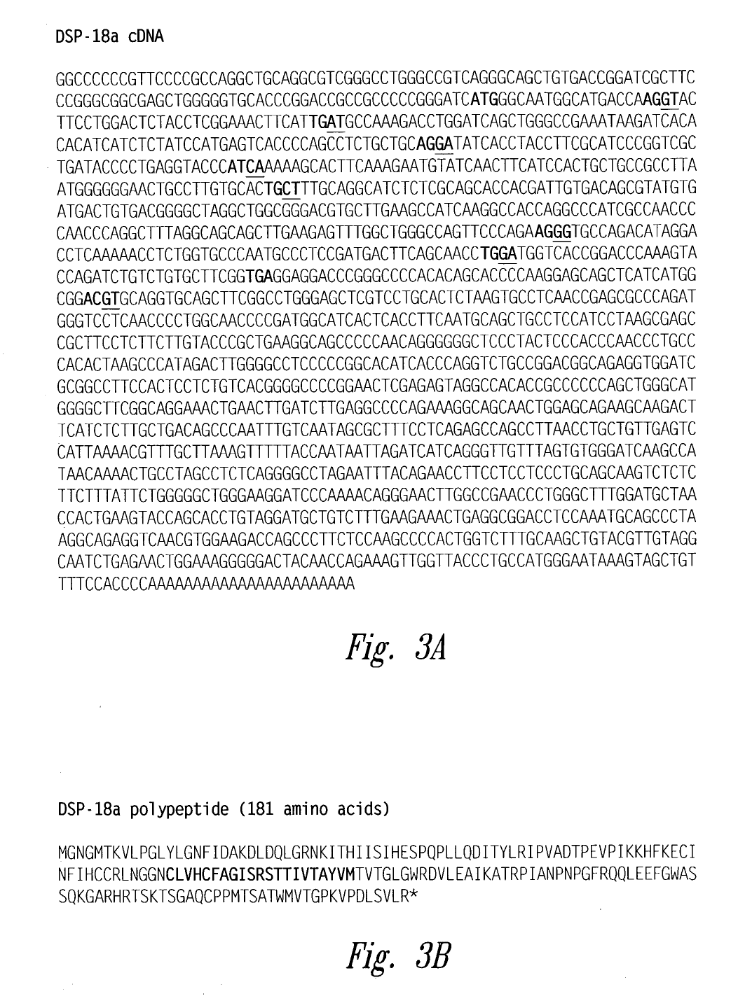

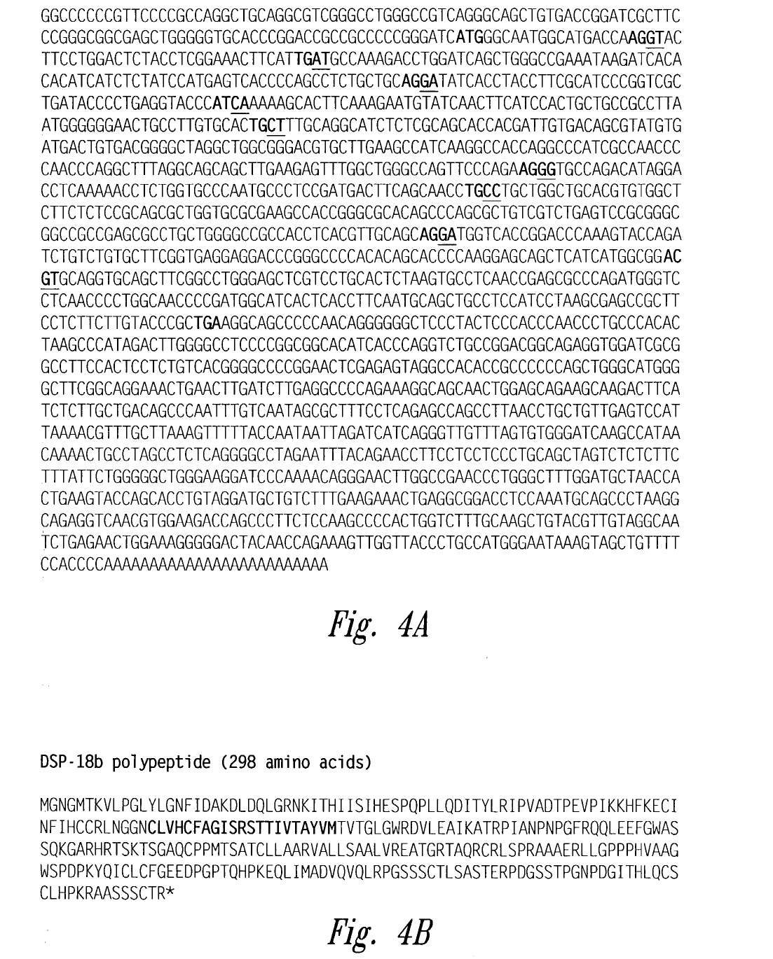

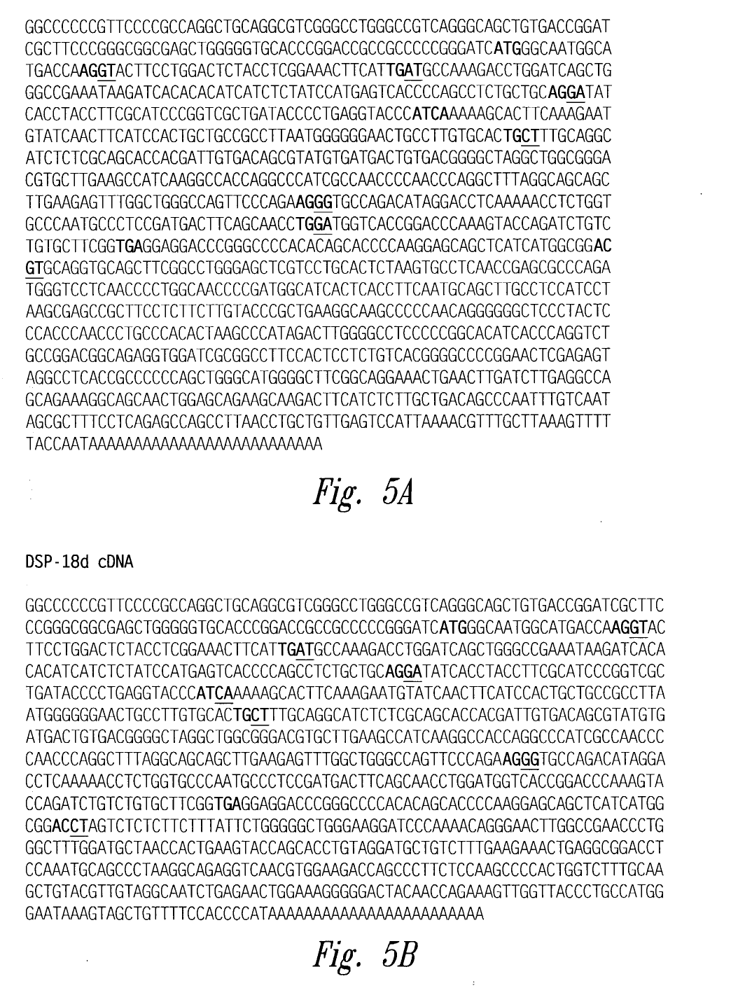

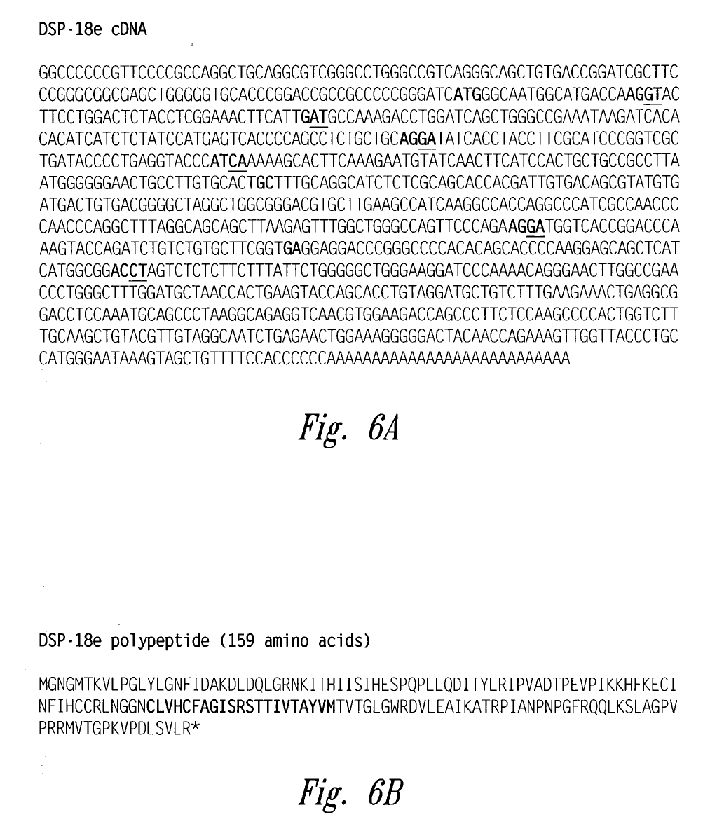

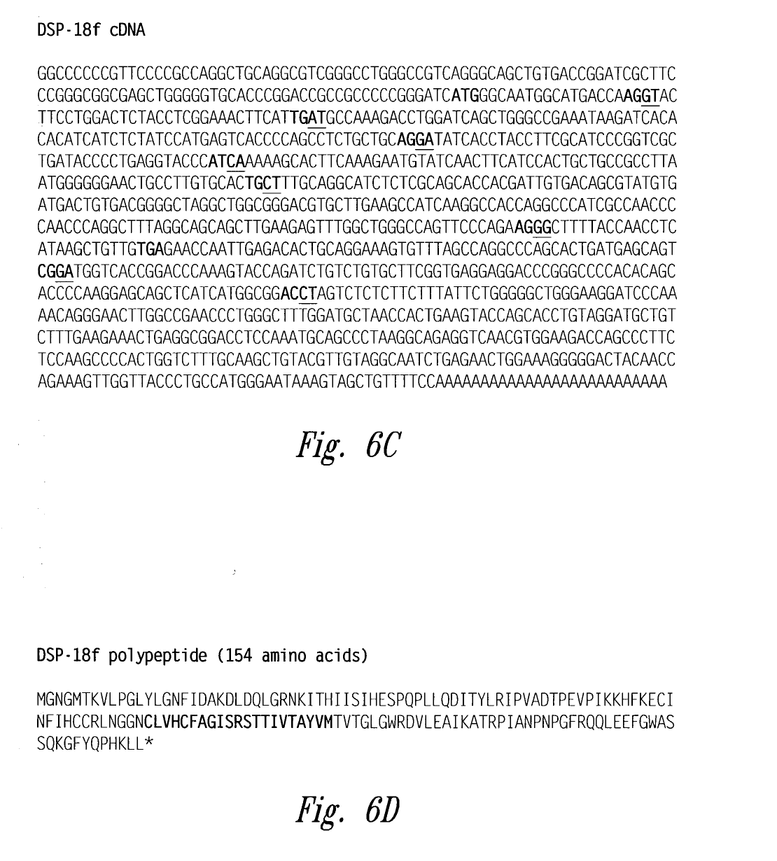

As noted above, the present invention is generally directed to compositions and methods for modulating (i.e., stimulating or inhibiting) cellular proliferative responses, in vitro and in vivo. In particular, the present invention provides DSP-18 dual-specificity phosphatases (prototypical DSP-18, FIG. 1, SEQ ID NOS: 13-14; and DSP-18 isoforms 18a-f, FIGS. 3-6; SEQ ID NOS:1-12), as well as variants thereof and antibodies that specifically bind DSP-18. Also provided herein are methods for using such DSP-18 compounds for screens, detection assays, and related therapeutic uses. The full length DSP-18 sequences of the present invention are distinct from, but are apparently related to, a proposed hypothetical open reading frame (ORF) for a phosphatase sequence previously disclosed in a genomic DNA sequence submission (GenBank Accession No. AL160175). This ORF, however, encodes a polypeptide that contains a four amino acid deletion from its hypothetical DSP active site domain relative to the DSP-18 sequences of the instant invention, as well as numerous additional major sequence deletions and additions, relative to the presently provided DSP-18 sequences. Despite its alleged identification as a DSP, no known polypeptide has phosphatase activity and has the four amino acid active site deletion of the ORF product of AL160175, and the existence of an actual, functional polypeptide having the amino acid sequence of the AL160175 ORF product and demonstrable phosphatase activity has never been confirmed. The full length DSP-18 sequences of the present invention are also distinct from, but apparently related to, polynucleotide and amino acid sequences disclosed in International Application No. PCT/US00/34736 (SGP008, therein SEQ ID NO: 8 (polynucleotide) and SEQ ID NO: 20 (polypeptide)) [SEQ ID NOS:39 and 31] and in International Application No. PCT/US01/30118 (69109, therein SEQ ID NOs: 1, 3, and 13 (polynucleotide) and SEQ ID NOs: 2 and 12 (polypeptides)) [SEQ ID NOS:42, 40, 41, and 32-33, respectively].

DSP-18 Polypeptides and Polynucleotides

As used herein, the term “DSP-18 polypeptide” refers to a polypeptide that comprises a DSP-18 amino acid sequence as provided herein (SEQ ID NOS:2, 4, 6, 8, 10, 12 or 14) or a variant of such a sequence. Such DSP-18 polypeptides are capable of dephosphorylating both tyrosine and threonine/serine residues in a DSP-18 substrate, with an activity that is not substantially diminished relative to that of a full length native DSP-18. DSP-18 substrates include activated (i.e., phosphorylated) MAP-kinases. Other substrates may be identified using substrate trapping mutants, as described herein, and include polypeptides having one or more phosphorylated tyrosine, threonine and/or serine residues.

DSP-18 polypeptide variants within the scope of the present invention may contain one or more substitutions, deletions, additions and/or insertions. For certain DSP-18 variants, the ability of the variant to dephosphorylate tyrosine and threonine residues within a DSP-18 substrate is not substantially diminished. The ability of such a DSP-18 variant to dephosphorylate tyrosine and threonine residues within a DSP-18 substrate may be enhanced or unchanged, relative to a native DSP-18, or may be diminished by less than 50%, less than 40%, preferably less than 30% or 25%, and more preferably less than 20%, relative to native DSP-18. Such variants may be identified using the representative assays provided herein.

Also contemplated by the present invention are modified forms of DSP-18 in which a specific function is disabled. For example, such proteins may be constitutively active or inactive, or may display altered binding or catalytic properties. Such altered proteins may be generated using well known techniques, and the altered function confirmed using screens such as those provided herein. Certain modified DSP-18 polypeptides are known as “substrate trapping mutants.” Such polypeptides retain the ability to bind a substrate (i.e., Kₘ is not substantially diminished), but display a reduced (e.g., decreased in a statistically significant manner) ability to dephosphorylate a substrate (i.e., k_{cat} is reduced, preferably to less than 1 per minute). Further, the stability of the substrate trapping mutant/substrate complex should not be substantially diminished, relative to the stability of a DSP-18/substrate complex. Complex stability may be assessed based on the association constant (Kₐ). Determination of Kₘ, k_{cat} and Kₐ may be readily accomplished using standard techniques known in the art (see, e.g., WO 98/04712; WO 00/75339; Lehninger, Biochemistry, 1975 Worth Publishers, NY) and assays provided herein. DSP-18 substrate trapping mutants may be generated, for example, by modifying a DSP-18 (SEQ ID NOS:2, 4, 6, 8, 10, 12 or 14) with an amino acid substitution at position 72 or position 103 (e.g., by replacing the amino acid aspartate at position 72 with an alanine residue, or by replacing the cysteine at residue 103 with a serine). Substrate trapping mutants may be used, for example, to identify substrates of DSP-18. Briefly, the modified DSP-18 may be contacted with a candidate substrate (alone or within a mixture of proteins, such as a cell extract) to permit the formation of a substrate/DSP-18 complex. The complex may then be isolated by conventional techniques to permit the isolation and characterization of substrate. The preparation and use of substrate trapping mutants is described, for example, within PCT Publication Nos. WO 98/04712 and WO 00/75339.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes.

In general, modifications may be more readily made in non-critical regions, which are regions of the native sequence that do not substantially change the activity of DSP-18. Non-critical regions may be identified by modifying the DSP-18 sequence in a particular region and assaying the ability of the resulting variant in a phosphatase assay, as described herein. Preferred sequence modifications are made so as to retain the DSP-18 active site domain, as described herein and as depicted in the drawings. Within certain preferred embodiments, such modifications affect interactions between DSP-18 and cellular components other than DSP-18 substrates. However, substitutions may also be made in critical regions of the native protein, provided that the resulting variant substantially retains the ability to stimulate substrate dephosphorylation. Within certain embodiments, a variant contains substitutions, deletions, additions, and/or insertions at no more than 50%, preferably no more than 45%, 40%, 35%, 30%, 25%, 20%, or 17.5%, still more preferably no more than 15%, 12%, 10%, 8%, 5%, 4%, 3%, 2%, or 1% of the amino acid residues.

Variants may also (or alternatively) be modified by, for example, the deletion or addition of amino acids that have minimal influence on the activity of the polypeptide. In particular, variants may contain additional amino acid sequences at the amino and/or carboxy termini. Such sequences may be used, for example, to facilitate purification or detection of the polypeptide.

DSP-18 polypeptides may be prepared using any of a variety of well known techniques. Recombinant polypeptides encoded by DNA sequences as described below may be readily prepared from the DNA sequences using any of a variety of expression vectors known to those having ordinary skill in the art. Expression may be achieved in any appropriate host cell that has been transformed or transfected with an expression vector containing a DNA molecule that encodes a recombinant polypeptide. Suitable host cells include prokaryotes, yeast and higher eukaryotic cells (including mammalian cells). Forms that differ in glycosylation may be generated by varying the host cell or by post-isolation processing. Supernatants from suitable host/vector systems that secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Portions and other variants having any number of amino acids fewer than about 100, 95, 90, 85, 80, 75, 70, 65, 60 or 55 amino acids, and generally any number of amino acids fewer than about 50 amino acids (e.g., 2, 3, 4, 5, 6, 7, 8, 9, 10, 11, 12, 13, 14, 15, 16, 17, 18, 19, 20, 21-25, 26-30, 31-35, 36-40, 41-45, or 46-49 amino acids), may also be generated by synthetic procedures, using techniques well known to those having ordinary skill in the art. For example, such polypeptides may be synthesized using any of the commercially available solid-phase techniques, such as the Merrifield solid-phase synthesis method, by which amino acids are sequentially added to a growing amino acid chain. See Merrifield, J. Am. Chem. Soc. 85:2149-2146, 1963. Equipment for automated synthesis of polypeptides is commercially available from suppliers such as Perkin-Elmer, Inc., Applied BioSystems Division (Foster City, Calif.), and may be operated according to the manufacturer's instructions.

A “DSP-18 polynucleotide” is any polynucleotide that encodes at least a portion of a DSP-18 polypeptide or a variant thereof, or that is complementary to such a polynucleotide. Preferred polynucleotides comprise at least 15 consecutive nucleotides, preferably at least 30, 35, 40, 50, 55, or 60 consecutive nucleotides, and in other preferred embodiments at least 70, 75, 80, 90, 100, 110, 120, 125, or 130 consecutive nucleotides, and in other preferred embodiments at least 136, 140, 144, 147, 150, 155, 160, or 170 consecutive nucleotides, and in other preferred embodiments at least 180, 190, 200, 225, 250, 275, 300, 325, 350, 375, 400, 405, 408, 410, 420, 425, 441, 445, 450, 475, 500, 525, 541, 545, 550, 575, 600, 700, or 800 consecutive nucleotides that include sequences which encode a DSP-18 polypeptide, or that are complementary to such a sequence. Certain polynucleotides encode a DSP-18 polypeptide; others may find use as probes, primers, or antisense oligonucleotides, as described below. Polynucleotides may be single-stranded (coding or antisense) or double-stranded, and may be DNA (genomic, cDNA, or synthetic) or RNA molecules. Additional coding or non-coding sequences may, but need not, be present within a polynucleotide of the present invention, and a polynucleotide may, but need not, be linked to other molecules and/or support materials.

DSP-18 polynucleotides may comprise a native sequence (i.e., an endogenous DSP-18 sequence or a portion or splice variant thereof) or may comprise a variant of such a sequence. Polynucleotide variants may contain one or more substitutions, additions, deletions, and/or insertions such that the activity of the encoded polypeptide is not substantially diminished, as described above. The effect on the activity of the encoded polypeptide may generally be assessed as described herein. Variants preferably exhibit at least about 70% identity, more preferably at least about 75%, 80%, 85%, or 88% identity and most preferably at least about 90%, 92%, 95%, 96%, 98%, or 99% identity to a polynucleotide sequence that encodes a native DSP-18 or a portion thereof. The percent identity may be readily determined by comparing sequences using computer algorithms well known to those having ordinary skill in the art, such as Align or the BLAST algorithm (Altschul, J. Mol. Biol. 219:555-565, 1991; Henikoff and Henikoff, Proc. Natl. Acad. Sci. USA 89:10915-10919, 1992), which is available at the NCBI website (http://www/ncbi.nlm.nih.gov/cgi-bin/BLAST). Default parameters may be used.

Certain variants are substantially homologous to a native gene. Such polynucleotide variants are capable of hybridizing under moderately stringent conditions to a naturally occurring DNA or RNA sequence encoding a native DSP-18 (or a complementary sequence). In a preferred embodiment of the invention, a polynucleotide that detectably hybridizes under moderately stringent conditions to a DSP-18 polynucleotide comprises a nucleotide sequence other than a sequence found in polynucleotides as disclosed in International Application No. PCT/US00/34736 (SGP008, therein SEQ ID NO: 8) [SEQ ID NO:39] and in International Application No. PCT/US01/30118 (69109, therein SEQ ID NOs: 1, 3, and 13) [SEQ ID NOS:42, 40, and 41]. A polynucleotide that detectably hybridizes under moderately stringent conditions may have a nucleotide sequence that includes at least 10 consecutive nucleotides, more preferably 15, 20, 25, 30, 35, 40, 45, 50, 55, 57, 58, 59, 60, 70, 72, 73, 75, 80, 85, or 90 consecutive nucleotides, more preferably 100, 120, 123, 126, 127, 130, 140, 160, 180, 200, 220, 240, 260, 280, or 300 consecutive nucleotides, and still more preferably 325, 350, 375, 400, 440, 450, 460, 465, 480, 500, 525, 550, 580, 600, 625, 650, 675, 700, 750, 800, 850 or 870 consecutive nucleotides complementary to a DSP-18 polynucleotide. Preferably, the polynucleotide detectably hybridizes to a polynucleotide having a sequence that is complementary to nucleotides located at positions (i) 553-660 as set forth in SEQ ID NO:1; (ii) 553-1011 as set forth in SEQ ID NO:3; (iii) 553-660 as set forth in SEQ ID NO:5; (iv) 553-660 as set forth in SEQ ID NO:7; (v) 523-594 as set forth in SEQ ID NO:9; or (vi) 553-579 as set forth in SEQ ID NO:11.

Suitable moderately stringent conditions include, for example, pre-washing in a solution of 5×SSC, 0.5% SDS, 1.0 mM EDTA (pH 8.0); hybridizing at 50° C.-70° C., 5×SSC for 1-16 hours; followed by washing once or twice at 22-65° C. for 20-40 minutes with one or more each of 2×, 0.5× and 0.2×SSC containing 0.05-0.1% SDS. For additional stringency, conditions may include a wash in 0.1×SSC and 0.1% SDS at 50-60° C. for 15 minutes. As known to those having ordinary skill in the art, variations in stringency of hybridization conditions may be achieved by altering the time, temperature, and/or concentration of the solutions used for pre-hybridization, hybridization, and wash steps. Suitable conditions may also depend in part on the particular nucleotide sequences of the probe used, and of the blotted, proband nucleic acid sample. Accordingly, it will be appreciated that suitably stringent conditions can be readily selected without undue experimentation when a desired selectivity of the probe is identified, based on its ability to hybridize to one or more certain proband sequences while not hybridizing to certain other proband sequences.

Persons having ordinary skill in the art will also readily appreciate that, as a result of the degeneracy of the genetic code, many nucleotide sequences may encode a polypeptide as described herein. Some of these polynucleotides bear minimal homology to the nucleotide sequence of any native gene. Nonetheless, polynucleotides that vary due to differences in codon usage are specifically contemplated by the present invention.

Polynucleotides may be prepared using any of a variety of techniques. For example, a polynucleotide may be amplified from cDNA prepared from a suitable cell or tissue type, such as human skeletal muscle. Such polynucleotides may be amplified via polymerase chain reaction (PCR). For this approach, sequence-specific primers may be designed based on the sequences provided herein, and may be purchased or synthesized.

An amplified portion may be used to isolate a full length gene from a suitable library (e.g., human skeletal muscle cDNA) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well known techniques. A bacterial or bacteriophage library may then be screened by hybridizing filters containing denatured bacterial colonies (or lawns containing phage plaques) with the labeled probe (see, e.g., Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. Clones may be analyzed to determine the amount of additional sequence by, for example, PCR using a primer from the partial sequence and a primer from the vector. Restriction maps and partial sequences may be generated to identify one or more overlapping clones. A full-length cDNA molecule can be generated by ligating suitable fragments, using well known techniques.

Alternatively, numerous amplification techniques are known in the art for obtaining a full length coding sequence from a partial cDNA sequence. Within such techniques, amplification is generally performed via PCR. One such technique is known as “rapid amplification of cDNA ends” or RACE. This technique involves the use of an internal primer and an external primer, which hybridizes to a polyA region or vector sequence, to identify sequences that are 5′ and 3′ of a known sequence. Any of a variety of commercially available kits may be used to perform the amplification step. Primers may be designed using, for example, software well known in the art. Primers (or oligonucleotides for other uses contemplated herein, including, for example, probes and antisense oligonucleotides) are preferably 15, 16, 17, 18, 19, 20, 21, 22, 23, 24, 25, 26, 27, 28, 29, 30, 31 or 32 nucleotides in length, have a GC content of at least 40% and anneal to the target sequence at temperatures of about 54° C. to 72° C. The amplified region may be sequenced as described above, and overlapping sequences assembled into a contiguous sequence. Certain oligonucleotides contemplated by the present invention may, for some preferred embodiments, have lengths of 33-35, 35-40, 41-45, 46-50, 56-60, 61-70, 71-80, 81-90 or more nucleotides.

A number of cDNA sequences encoding prototypical DSP-18 (DSP-18pr) and DSP-18 isoforms DSP-18a-f are provided in FIGS. 1 and 3-6 (SEQ ID NOS:1, 3, 5, 7, 9, 11, 13). The predicted full length amino acid sequences are also provided in these Figures (SEQ ID NOS:2, 4, 6, 8, 10, 12 and 14). The DSP-18 active site as found in DSP-18 isoforms a-f, CLVHCFAGISRSTTIVTAYVM [SEQ ID NO:17] is located at residues 84-104 of SEQ ID NO:2. Sequence information immediately adjacent to this site was used to design 5′ and 3′ RACE reactions with human testis cDNA to identify a cDNA molecule with an open reading frame of 543 base pairs, encoding a 181 amino acid protein (FIG. 3). This protein is referred to as dual specificity phosphatase-18a, DSP-18a, and is a distinct but related isoform of other DSP-18 sequences disclosed herein. Although apparently expressed in a wide variety of human tissues, DSP-18a shows higher apparent message levels in human testis, kidney and brain, relative to other tissues

Polynucleotide variants of DSP-18, including any DSP-18 isoform described herein, may generally be prepared by any method known in the art, including, for example, solid phase chemical synthesis. Modifications in a polynucleotide sequence may also be introduced using standard mutagenesis techniques, such as oligonucleotide-directed site-specific mutagenesis. Alternatively, RNA molecules may be generated by in vitro or in vivo transcription of DNA sequences encoding DSP-18, or a portion thereof, provided that the DNA is incorporated into a vector with a suitable RNA polymerase promoter (such as T7 or SP6). Certain polynucleotides may be used to prepare an encoded polypeptide, as described herein. In addition, or alternatively, a polynucleotide may be administered to a patient such that the encoded polypeptide is generated in vivo.

A polynucleotide that is complementary to at least a portion of a coding sequence (e.g., an antisense polynucleotide or a ribozyme) may also be used as a probe or primer, or to modulate gene expression. Identification of oligonucleotides and ribozymes for use as antisense agents, and DNA encoding genes for their targeted delivery, involve methods well known in the art. For example, the desirable properties, lengths, and other characteristics of such oligonucleotides are well known. Antisense oligonucleotides are typically designed to resist degradation by endogenous nucleolytic enzymes by using such linkages as phosphorothioate, methylphosphonate, sulfone, sulfate, ketyl, phosphorodithioate, phosphoramidate, phosphate esters, and other such linkages (see, e.g., Agrwal et al., Tetrahedron Lett. 28:3539-3542 (1987); Miller et al., J. Am. Chem. Soc. 93:6657-6665 (1971); Stec et al., Tetrahedron Lett. 26:2191-2194 (1985); Moody et al., Nucleic Acids Res. 12:4769-4782 (1989); Uznanski et al., Nucleic Acids Res. (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein, In: Oligodeoxynucleotides. Antisense Inhibitors of Gene Expression, Cohen, ed., Macmillan Press, London, pp. 97-117 (1989); Jager et al., Biochemistry 27:7237-7246 (1988)).

Antisense polynucleotides are oligonucleotides that bind in a sequence-specific manner to nucleic acids such as mRNA or DNA. When bound to mRNA that has complementary sequences, antisense prevents translation of the mRNA (see, e.g., U.S. Pat. No. 5,168,053 to Altman et al.; U.S. Pat. No. 5,190,931 to Inouye, U.S. Pat. No. 5,135,917 to Burch; U.S. Pat. No. 5,087,617 to Smith and Clusel et al. (1993) Nucleic Acids Res. 21:3405-3411, which describes dumbbell antisense oligonucleotides). Triplex molecules refer to single DNA strands that bind duplex DNA forming a colinear triplex molecule, thereby preventing transcription (see, e.g., U.S. Pat. No. 5,176,996 to Hogan et al., which describes methods for making synthetic oligonucleotides that bind to target sites on duplex DNA).

Particularly useful antisense nucleotides and triplex molecules are molecules that are complementary to or bind the sense strand of DNA or mRNA that encodes a DSP-18 polypeptide or a protein mediating any other process related to expression of endogenous DSP-18, such that inhibition of translation of mRNA encoding the DSP-18 polypeptide is effected. cDNA constructs that can be transcribed into antisense RNA may also be introduced into cells or tissues to facilitate the production of antisense RNA. Antisense technology can be used to control gene expression through interference with binding of polymerases, transcription factors, or other regulatory molecules (see Gee et al., In Huber and Carr, Molecular and Immunologic Approaches, Futura Publishing Co. (Mt. Kisco, N.Y.; 1994)). Alternatively, an antisense molecule may be designed to hybridize with a control region of a DSP-18 gene (e.g., promoter, enhancer or transcription initiation site) and block transcription of the gene, or to block translation by inhibiting binding of a transcript to ribosomes.

The present invention also contemplates DSP-18-specific ribozymes. A ribozyme is an RNA molecule that specifically cleaves RNA substrates, such as mRNA, resulting in specific inhibition or interference with cellular gene expression. At least five known classes of ribozymes are involved in the cleavage and/or ligation of RNA chains. Ribozymes can be targeted to any RNA transcript and can catalytically cleave such transcripts (see, e.g., U.S. Pat. Nos. 5,272,262; 5,144,019; and 5,168,053, 5,180,818, 5,116,742 and 5,093,246 to Cech et al.). Any DSP-18 mRNA-specific ribozyme, or a nucleic acid encoding such a ribozyme, may be delivered to a host cell to effect inhibition of DSP-18 gene expression. Ribozymes may therefore be delivered to the host cells by DNA encoding the ribozyme linked to a eukaryotic promoter, such as a eukaryotic viral promoter, such that upon introduction into the nucleus, the ribozyme will be directly transcribed.

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine and wybutosine, as well as acetyl-methyl-, thio- and other modified forms of adenine, cytidine, guanine, thymine, and uridine.

Nucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives, and cosmids. Vectors of particular interest include expression vectors, replication vectors, probe generation vectors, and sequencing vectors. In general, a suitable vector contains an origin of replication functional in at least one organism, convenient restriction endonuclease sites, and one or more selectable markers. Other elements will depend upon the desired use, and will be apparent to those having ordinary skill in the art. For example, the invention contemplates the use of DSP-18 nucleotide sequences in the preparation of expression vectors including vectors for the overexpression of a DSP-18 polypeptide in vivo; the invention also contemplates the generation of DSP-18 “knock-out” animals and cells (e.g., cells, cell clones, lines or lineages, or organisms in which expression of a DSP-18 is fully or partially compromised).

Within certain embodiments, polynucleotides may be formulated so as to permit entry into a cell of a mammal, and expression therein. Such formulations are particularly useful for therapeutic purposes, as described below. Those having ordinary skill in the art will appreciate that there are many ways to achieve expression of a polynucleotide in a target cell, and any suitable method may be employed. For example, a polynucleotide may be incorporated into a viral vector using well known techniques. A viral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those having ordinary skill in the art.

Other formulations for therapeutic purposes include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Within other aspects, a DSP-18 promoter may be isolated using standard techniques. The present invention provides nucleic acid molecules comprising such a promoter sequence or one or more cis- or trans-acting regulatory elements thereof. Such regulatory elements may enhance or suppress expression of DSP-18. A 5′ flanking region may be generated using standard techniques, based on the genomic sequence provided herein. If necessary, additional 5′ sequences may be generated using PCR-based or other standard methods. The 5′ region may be subcloned and sequenced using standard methods. Primer extension and/or RNase protection analyses may be used to verify the transcriptional start site deduced from the cDNA.

To define the boundary of the promoter region, putative promoter inserts of varying sizes may be subcloned into a heterologous expression system containing a suitable reporter gene without a promoter or enhancer. Suitable reporter genes may include genes encoding luciferase, beta-galactosidase, chloramphenicol acetyl transferase, secreted alkaline phosphatase or the Green Fluorescent Protein gene. Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of DSP-18 expression. In general, the construct with the minimal 5′ flanking region showing the highest level of expression of reporter gene is identified as the promoter. Such promoter regions may be linked to a reporter gene and used to evaluate agents for the ability to modulate DSP-18 transcription.

Once a functional promoter is identified, cis- and trans-acting elements may be located. Cis-acting sequences may generally be identified based on homology to previously characterized transcriptional motifs. Point mutations may then be generated within the identified sequences to evaluate the regulatory role of such sequences. Such mutations may be generated using site-specific mutagenesis techniques or a PCR-based strategy. The altered promoter is then cloned into a reporter gene expression vector, as described above, and the effect of the mutation on reporter gene expression is evaluated.

The present invention also contemplates the use of allelic variants of DSP-18, as well as DSP-18 sequences from other organisms. Such sequences may generally be identified based upon similarity to the sequences provided herein (e.g., using hybridization techniques) and based upon the presence of DSP-18 activity, using an assay provided herein.

In general, polypeptides and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

Assays for Detecting DSP-18 Activity

According to the present invention, substrates of or DSP-18 may include full length tyrosine phosphorylated proteins and polypeptides as well as fragments (e.g., portions), derivatives or analogs thereof that can be phosphorylated at a tyrosine residue and that may, in certain preferred embodiments, also be able to undergo phosphorylation at a serine or a threonine residue. Such fragments, derivatives and analogs include any naturally occurring or artificially engineered DSP-18 substrate polypeptide that retains at least the biological function of interacting with a DSP-18 as provided herein, for example by forming a complex with a DSP-18. A fragment, derivative or analog of a DSP-18 substrate polypeptide, including substrates that are fusion proteins, may be (i) one in which one or more of the amino acid residues are substituted with a conserved or non-conserved amino acid residue (preferably a conserved amino acid residue), and such substituted amino acid residue may or may not be one encoded by the genetic code, or (ii) one in which one or more of the amino acid residues includes a substituent group, or (iii) one in which the substrate polypeptide is fused with another compound, such as a compound to increase the half-life of the polypeptide (e.g., polyethylene glycol) or a detectable moiety such as a reporter molecule, or (iv) one in which additional amino acids are fused to the substrate polypeptide, including amino acids that are employed for purification of the substrate polypeptide or a proprotein sequence. Such fragments, derivatives, and analogs are deemed to be within the scope of those skilled in the art. In preferred embodiment, a MAP-kinase polypeptide is a substrate for use as provided herein.

Polypeptide variants of DSP-18 may be tested for DSP-18 activity using any suitable assay for MAP-kinase phosphatase activity. Such assays may be performed in vitro or within a cell-based assay. For example, a MAP-kinase may be obtained in inactive form from Upstate Biotechnology (Lake Placid, N.Y.; catalog number 14-198), for use as a DSP-18 substrate as provided herein. Phosphorylation of the MAP-kinase can be performed using well known techniques (such as those described by Zheng and Guan, J. Biol. Chem. 268:16116-16119, 1993) using the MAP-kinase kinase MEK-1 (available from Upstate Biotechnology; cat. no. 14-206).

For example, [³²P]-radiolabeled substrate (e.g., MAP-kinase) may be used for the kinase reaction, resulting in radiolabeled, activated MAP-kinase. A DSP-18 polypeptide may then be tested for the ability to dephosphorylate an activated MAP-kinase by contacting the DSP-18 polypeptide with the MAP-kinase under suitable conditions (e.g., Tris, pH 7.5, 1 mM EDTA, 1 mM dithiothreitol, 1 mg/mL bovine serum albumin for 10 minutes at 30° C.; or as described by Zheng and Guan, J. Biol. Chem. 268:16116-16119, 1993). Dephosphorylation of the MAP-kinase may be detected using any of a variety of assays, such as a coupled kinase assay (evaluating phosphorylation of a MAP-kinase substrate using any assay generally known in the art) or directly, based on (1) the loss of radioactive phosphate groups (e.g., by gel electrophoresis, followed by autoradiography); (2) the shift in electrophoretic mobility following dephosphorylation; (3) the loss of reactivity with an antibody specific for phosphotyrosine or phosphothreonine; or (4) a phosphoamino acid analysis of the MAP-kinase. Certain assays may generally be performed as described by Ward et al., Nature 367:651-654, 1994 or Alessi et al., Oncogene 8:2015-2020, 1993. In general, contact of 500 pg-50 ng of DSP-18 polypeptide with 100 ng-100 μg activated MAP-kinase should result in a detectable dephosphorylation of the MAP-kinase, typically within 20-30 minutes. Within certain embodiments, 0.01-10 units/ml (preferably about 0.1 units/ml, in which a unit is an amount sufficient to dephosphorylate 1 nmol substrate per minute) DSP-18 polypeptide may be contacted with 0.1-10 μM (preferably about 1 μM) activated MAP-kinase to produce a detectable dephosphorylation of a MAP-kinase. Preferably, a DSP-18 polypeptide results in dephosphorylation of a MAP-kinase or a phosphorylated substrate (such as a tyrosine-and/or serine-phosphorylated peptide) that is at least as great as the dephosphorylation observed in the presence of a comparable amount of native human DSP-18. It will be apparent that other substrates identified using a substrate trapping mutant as described herein may be substituted for the MAP-kinase within such assays.

Antibodes and Antigen-Binding Fragments

Also contemplated by the present invention are peptides, polypeptides, and other non-peptide molecules that specifically bind to a DSP-18. As used herein, a molecule is said to “specifically bind” to a DSP-18 if it reacts at a detectable level with DSP-18, but does not react detectably with peptides containing an unrelated sequence, or a sequence of a different phosphatase. Preferred binding molecules include antibodies, which may be, for example, polyclonal, monoclonal, single chain, chimeric, anti-idiotypic, or CDR-grafted immunoglobulins, or fragments thereof, such as proteolytically generated or recombinantly produced immunoglobulin F(ab′)₂, Fab, Fv, and Fd fragments. Certain preferred antibodies are those antibodies that inhibit or block DSP-18 activity within an in vitro assay, as described herein. Binding properties of an antibody to DSP-18 may generally be assessed using immunodetection methods including, for example, an enzyme-linked immunosorbent assay (ELISA), immunoprecipitation, immunoblotting and the like, which may be readily performed by those having ordinary skill in the art.

Methods well known in the art may be used to generate antibodies, polyclonal antisera or monoclonal antibodies that are specific for a DSP-18. Antibodies also may be produced as genetically engineered immunoglobulins (Ig) or Ig fragments designed to have desirable properties. For example, by way of illustration and not limitation, antibodies may include a recombinant IgG that is a chimeric fusion protein having at least one variable (V) region domain from a first mammalian species and at least one constant region domain from a second, distinct mammalian species. Most commonly, a chimeric antibody has murine variable region sequences and human constant region sequences. Such a murine/human chimeric immunoglobulin may be “humanized” by grafting the complementarity determining regions (CDRs) derived from a murine antibody, which confer binding specificity for an antigen, into human-derived V region framework regions and human-derived constant regions. Fragments of these molecules may be generated by proteolytic digestion, or optionally, by proteolytic digestion followed by mild reduction of disulfide bonds and alkylation. Alternatively, such fragments may also be generated by recombinant genetic engineering techniques.

As used herein, an antibody is said to be “immunospecific” or to “specifically bind” a DSP-18 polypeptide if it reacts at a detectable level with DSP-18, preferably with an affinity constant, Kₐ, of greater than or equal to about 10⁴ M⁻¹, more preferably of greater than or equal to about 10⁵ M⁻¹, more preferably of greater than or equal to about 10⁶ M⁻¹, and still more preferably of greater than or equal to about 10⁷ M⁻¹. Affinities of binding partners or antibodies can be readily determined using conventional techniques, for example, those described by Scatchard et al. (Ann. N.Y. Acad. Sci. USA 51:660 (1949)) or by surface plasmon resonance (BIAcore, Biosensor, Piscataway, N.J.). See, e.g., Wolff et al., Cancer Res. 53:2560-2565 (1993).

Antibodies may generally be prepared by any of a variety of techniques known to those having ordinary skill in the art. See, e.g., Harlow et al., Antibodies: A Laboratory Manual, Cold Spring Harbor Laboratory (1988). In one such technique, an animal is immunized with DSP-18 as an antigen to generate polyclonal antisera. Suitable animals include, for example, rabbits, sheep, goats, pigs, cattle, and may also include smaller mammalian species, such as mice, rats, and hamsters, or other species.

An immunogen may be comprised of cells expressing DSP-18, purified or partially purified DSP-18 polypeptides, or variants or fragments (e.g., peptides) thereof, or DSP-18 peptides. DSP-18 peptides may be generated by proteolytic cleavage or may be chemically synthesized. For instance, nucleic acid sequences encoding DSP-18 polypeptides are provided herein, such that those skilled in the art may routinely prepare these polypeptides for use as immunogens. Peptides may be chemically synthesized by methods as described herein and known in the art. Alternatively, peptides may be generated by proteolytic cleavage of a DSP-18 polypeptide, and individual peptides isolated by methods known in the art such as polyacrylamide gel electrophoresis or any number of liquid chromatography or other separation methods. Peptides useful as immunogens typically may have an amino acid sequence of at least 4 or 5 consecutive amino acids from a DSP-18 amino acid sequence such as those described herein, and preferably have at least 6, 7, 8, 9, 10, 11, 12, 14, 15, 16, 18, 19 or 20 consecutive amino acids of a DSP-18 polypeptide. Certain other preferred peptide immunogens may comprise 21-25, 26-30, 31-35, 36-40, 41-50 or more consecutive amino acids of a DSP-18 polypeptide sequence. Polypeptides or peptides useful for immunization may also be selected by analyzing the primary, secondary, and tertiary structure of DSP-18 according to methods known to those skilled in the art, in order to determine amino acid sequences more likely to generate an antigenic response in a host animal. See, e.g., Novotny, 1991 Mol. Immunol. 28:201-207; Berzofsky, 1985 Science 229:932-40.

Preparation of the immunogen for injection into animals may include covalent coupling of the DSP-18 polypeptide (or variant or fragment thereof), to another immunogenic protein, for example, a carrier protein such as keyhole limpet hemocyanin (KLH) or bovine serum albumin (BSA). In addition, the DSP-18 peptide, polypeptide, or DSP-18-expressing cells to be used as immunogen may be emulsified in an adjuvant. See, e.g., Harlow et al., Antibodies: A Laboratory Manual, Cold Spring Harbor Laboratory (1988). In general, after the first injection, animals receive one or more booster immunizations according to a preferred schedule that may vary according to, inter alia, the antigen, the adjuvant (if any) and/or the particular animal species. The immune response may be monitored by periodically bleeding the animal, separating the sera out of the collected blood, and analyzing the sera in an immunoassay, such as an ELISA or Ouchterlony diffusion assay, or the like, to determine the specific antibody titer. Once an antibody titer is established, the animals may be bled periodically to accumulate the polyclonal antisera. Polyclonal antibodies that bind specifically to the DSP-18 polypeptide or peptide may then be purified from such antisera, for example, by affinity chromatography using protein A, or the DSP-18 polypeptide, immobilized on a suitable solid support.

Monoclonal antibodies that specifically bind to DSP-18 polypeptides or fragments or variants thereof, and hybridomas, which are immortal eukaryotic cell lines, that produce monoclonal antibodies having the desired binding specificity, may also be prepared, for example, using the technique of Kohler and Milstein (Nature, 256:495-497; 1976, Eur. J. Immunol. 6:511-519 (1975)) and improvements thereto. An animal—for example, a rat, hamster, or preferably mouse-is immunized with a DSP-18 immunogen prepared as described above. Lymphoid cells that include antibody-forming cells, typically spleen cells, are obtained from an immunized animal and may be immortalized by fusion with a drug-sensitized myeloma (e.g., plasmacytoma) cell fusion partner, preferably one that is syngeneic with the immunized animal and that optionally has other desirable properties (e.g., inability to express endogenous Ig gene products). The lymphoid (e.g., spleen) cells and the myeloma cells may be combined for a few minutes with a membrane fusion-promoting agent, such as polyethylene glycol or a nonionic detergent, and then plated at low density on a selective medium that supports the growth of hybridoma cells, but not unfused myeloma cells. A preferred selection media is HAT (hypoxanthine, aminopterin, thymidine). After a sufficient time, usually about one to two weeks, colonies of cells are observed. Single colonies are isolated, and antibodies produced by the cells may be tested for binding activity to the DSP-18 polypeptide, or variant or fragment thereof. Hybridomas producing monoclonal antibodies with high affinity and specificity for a DSP-18 antigen are preferred. Hybridomas that produce monoclonal antibodies that specifically bind to a DSP-18 polypeptide or variant or fragment thereof are therefore contemplated by the present invention.

Monoclonal antibodies may be isolated from the supernatants of hybridoma cultures. An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Contaminants may be removed from the subsequently (usually within 1-3 weeks) harvested ascites fluid by conventional techniques, such as chromatography, gel filtration, precipitation, extraction, or the like. For example, antibodies may be purified by affinity chromatography using an appropriate ligand selected based on particular properties of the monoclonal antibody (e.g., heavy or light chain isotype, binding specificity, etc.). Examples of a suitable ligand, immobilized on a solid support, include Protein A, Protein G, an anti-constant region (light chain or heavy chain) antibody, an anti-idiotype antibody and a DSP-18 polypeptide or fragment or variant thereof.

Human monoclonal antibodies may be generated by any number of techniques with which those having ordinary skill in the art will be familiar. Such methods include but are not limited to, Epstein Barr Virus (EBV) transformation of human peripheral blood cells (e.g., containing B lymphocytes), in vitro immunization of human B cells, fusion of spleen cells from immunized transgenic mice carrying human immunoglobulin genes inserted by yeast artificial chromosomes (YAC), isolation from human immunoglobulin V region phage libraries, or other procedures as known in the art and based on the disclosure herein.

For example, one method for generating human monoclonal antibodies includes immortalizing human peripheral blood cells by EBV transformation. See, e.g., U.S. Pat. No. 4,464,456. An immortalized cell line producing a monoclonal antibody that specifically binds to a DSP-18 polypeptide (or a variant or fragment thereof) can be identified by immunodetection methods as provided herein, for example, an ELISA, and then isolated by standard cloning techniques. Another method to generate human monoclonal antibodies, in vitro immunization, includes priming human splenic B cells with antigen, followed by fusion of primed B cells with a heterohybrid fusion partner. See, e.g., Boerner et al., 1991 J. Immunol. 147:86-95.

Still another method for the generation of human DSP-18-specific monoclonal antibodies and polyclonal antisera for use in the present invention relates to transgenic mice. See, e.g., U.S. Pat. No. 5,877,397; Bruggemann et al., 1997 Curr. Opin. Biotechnol. 8:455-58; Jakobovits et al., 1995 Ann. N. Y. Acad. Sci. 764:525-35. In these mice, human immunoglobulin heavy and light chain genes have been artificially introduced by genetic engineering in germline configuration, and the endogenous murine immunoglobulin genes have been inactivated. See, e.g., Bruggemann et al., 1997 Curr. Opin. Biotechnol. 8:455-58. For example, human immunoglobulin transgenes may be mini-gene constructs, or transloci on yeast artificial chromosomes, which undergo B cell-specific DNA rearrangement and hypermutation in the mouse lymphoid tissue. See, Bruggemann et al., 1997 Curr. Opin. Biotechnol. 8:455-58. Human monoclonal antibodies specifically binding to DSP-18 may be obtained by immunizing the transgenic animals, fusing spleen cells with myeloma cells, selecting and then cloning cells producing antibody, as described above. Polyclonal sera containing human antibodies may also be obtained from the blood of the immunized animals.

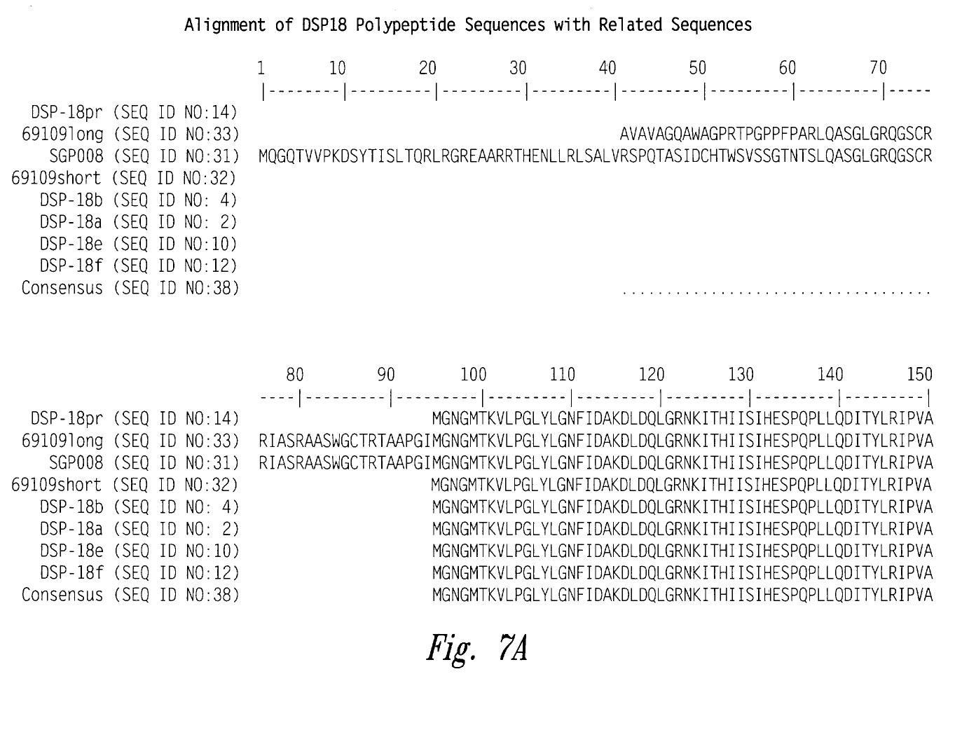

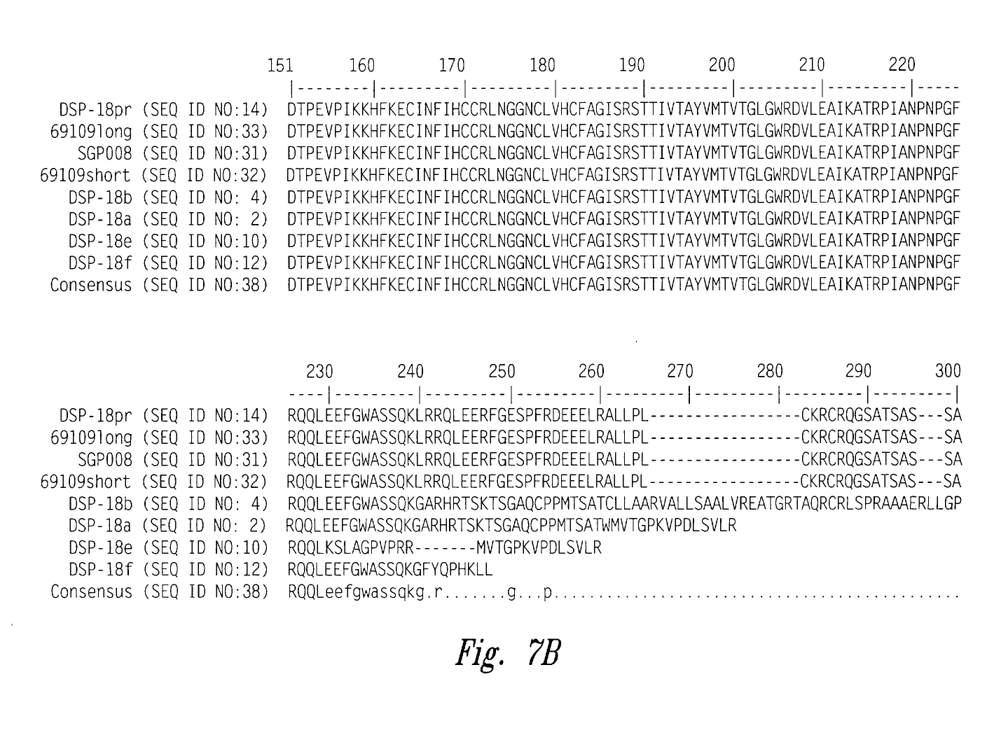

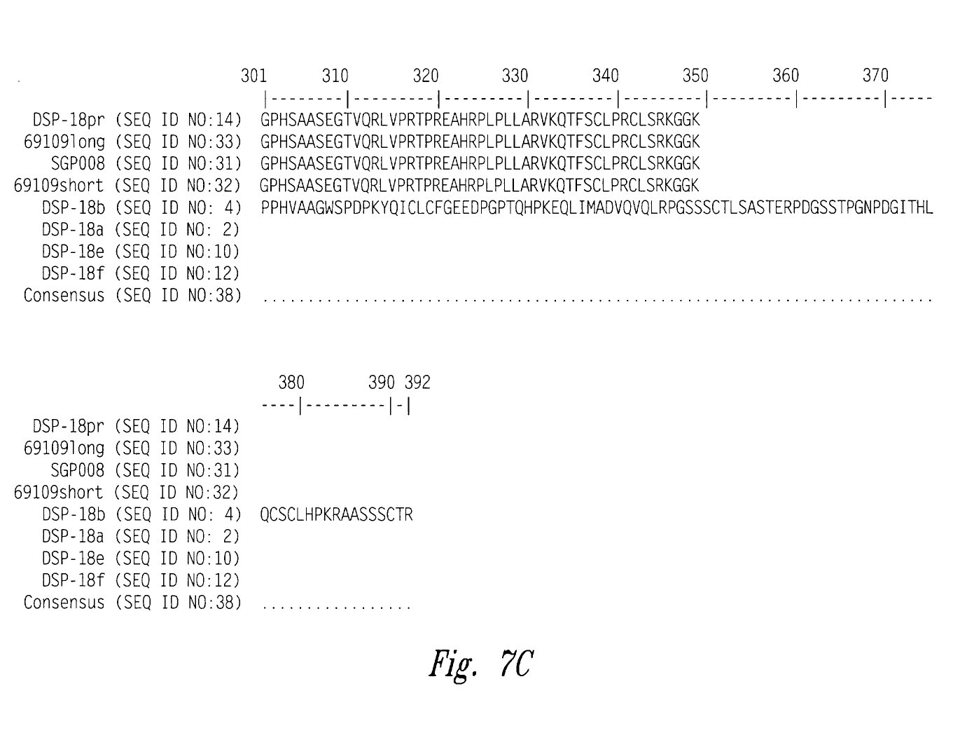

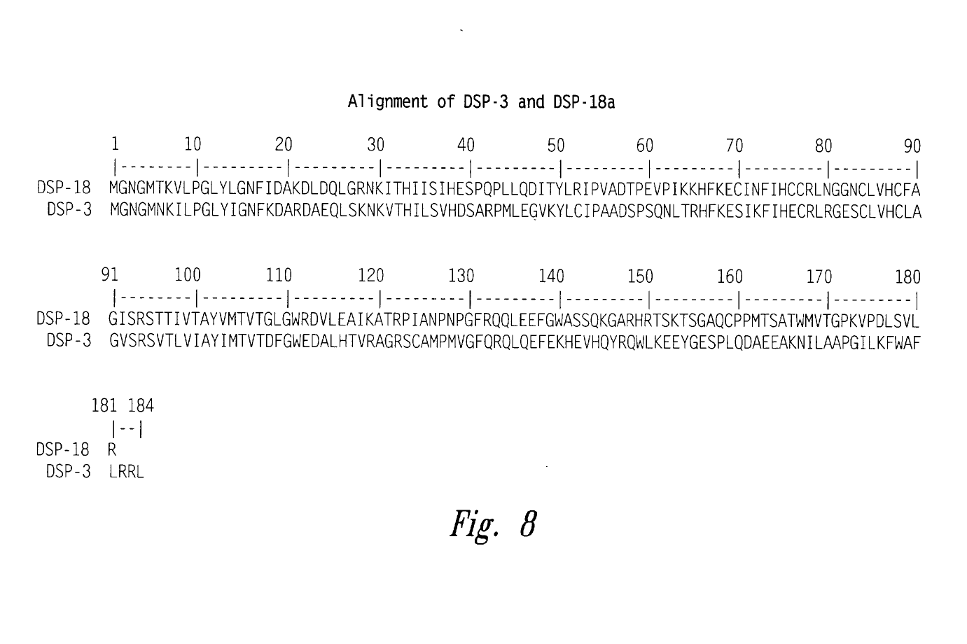

Antibodies that specifically bind DSP-18, variants and fragments thereof, and that do not specifically bind to dual specificity phosphatases as disclosed in International Application No. PCT/US00/34736 (SGP008, therein SEQ ID NO:20) [SEQ ID NO:31 herein] or in International Application No. PCT/USOI/30118 (69109 polypeptides, therein SEQ ID NOs: 2 and 12) [SEQ ID NOs:32 and 33 herein] may be selected by methods disclosed herein and known in the art. For example, antibodies specific for DSP-18 may be isolated from antisera collected from animals immunized with a DSP-18 polypeptide, fragment, or peptide as provided herein (including, e.g., a DSP-18 peptide immunogen) by absorbing the antisera with SGP008 or 69109 polypeptides to remove antibodies that bind to a shared antigenic determinant. Antibodies that bind only to DSP-18 and not to SGP008 or 69109 polypeptides may be identified by including these polypeptides in screening assays and selecting the antibodies that specifically bind only to the DSP-18 polypeptide. Antibodies specific for a DSP-18 polypeptide may also be selected by affinity purification using a matrix to which DSP-18 fragments or peptides that do not have shared sequences with any of the SGP008 or 69109 polypeptides have been attached. Alternatively, the unique DSP-18 fragments or peptides may be used as immunogens. A person skilled in the art may readily determine which DSP-18 peptide sequences are not shared with SGP008 or 69109 polypeptides by aligning the polypeptides according to methods disclosed herein and known in the art (also see FIG. 7). For example, fragments or peptides derived from amino acid residues located at positions 146-181 of DSP-18a, c, and d [SEQ ID NO:2, 6, and 8], 146-298 of DSP-18b [SEQ ID NO:4], 136-159 of DSP-18e [SEQ ID NO: 10], or 146-154 of DSP-18f [SEQ ID NO: 12] may be attached to a matrix for affinity chromatography or used as immunogens, or may otherwise comprise DSP-18 polypeptide antigenic determinants to which certain isolated antibodies may specifically bind, according to the present disclosure.

An antibody of the present invention may specifically bind to an antigenic determinant of a DSP-18 polypeptide that comprises at least 3, preferably at least 4 or 5, and more preferably at least 6, 7, 8, 9, 10, 11, 12, 13, 14, 15, 16-20, 21-25, 26-30, 31-40 or more consecutive amino acids located at or within positions 146-181 of DSP-18a, c, and d [SEQ ID NO:2, 6, and 8], 146-298 ofDSP-18b [SEQ ID NO:4], 136-159 of DSP-18e [SEQ ID NO: 10], or 146-154 of DSP-18f [SEQ ID NO: 12]. In another embodiment of the invention, a DSP-18 antibody specifically binds to an antigenic determinant that is formed by the three-dimensional conformation of the DSP-18 polypeptide. Such a conformational antigenic determinant may or may not include consecutive amino acids. In a preferred embodiment of the invention, an antibody that specifically binds to a DSP-18 polypeptide binds to an antigenic determinant that comprises at least one amino acid located at the carboxyl end of the DSP-18 polypeptide, preferably located at positions 146-181 of DSP-18a [SEQ ID NO:2], 146-298 of DSP-18b [SEQ ID NO:4], 136-159 of DSP-18e [SEQ ID NO: 10], or 136-154 of DSP-18f [SEQ ID NO: 12].

Chimeric antibodies, specific for a DSP-18, including humanized antibodies, may also be generated according to the present invention. A chimeric antibody has at least one constant region domain derived from a first mammalian species and at least one variable region domain derived from a second, distinct mammalian species. See, e.g., Morrison et al., 1984, Proc. Natl. Acad. Sci. USA, 81:6851-55. In preferred embodiments, a chimeric antibody may be constructed by cloning the polynucleotide sequence that encodes at least one variable region domain derived from a non-human monoclonal antibody, such as the variable region derived from a murine, rat, or hamster monoclonal antibody, into a vector containing a nucleic acid sequence that encodes at least one human constant region. See, e.g., Shin et al., 1989 Methods Enzymol. 178:459-76; Walls et al., 1993 Nucleic Acids Res. 21:2921-29. By way of example, the polynucleotide sequence encoding the light chain variable region of a murine monoclonal antibody may be inserted into a vector containing a nucleic acid sequence encoding the human kappa light chain constant region sequence. In a separate vector, the polynucleotide sequence encoding the heavy chain variable region of the monoclonal antibody may be cloned in frame with sequences encoding the human IgG1 constant region. The particular human constant region selected may depend upon the effector functions desired for the particular antibody (e.g., complement fixing, binding to a particular Fe receptor, etc.). Another method known in the art for generating chimeric antibodies is homologous recombination (e.g., U.S. Pat. No. 5,482,856). Preferably, the vectors will be transfected into eukaryotic cells for stable expression of the chimeric antibody.

A non-human/human chimeric antibody may be further genetically engineered to create a “humanized” antibody. Such a humanized antibody may comprise a plurality of CDRs derived from an immunoglobulin of a non-human mammalian species, at least one human variable framework region, and at least one human immunoglobulin constant region. Humanization may in certain embodiments provide an antibody that has decreased binding affinity for a DSP-18 when compared, for example, with either a non-human monoclonal antibody from which a DSP-18 binding variable region is obtained, or a chimeric antibody having such a V region and at least one human C region, as described above. Useful strategies for designing humanized antibodies may therefore include, for example by way of illustration and not limitation, identification of human variable framework regions that are most homologous to the non-human framework regions of the chimeric antibody. Without wishing to be bound by theory, such a strategy may increase the likelihood that the humanized antibody will retain specific binding affinity for a DSP-18, which in some preferred embodiments may be substantially the same affinity for a DSP-18 polypeptide or variant or fragment thereof, and in certain other preferred embodiments may be a greater affinity for DSP-18. See, e.g., Jones et al., 1986 Nature 321:522-25; Riechmann et al., 1988 Nature 332:323-27. Designing such a humanized antibody may therefore include determining CDR loop conformations and structural determinants of the non-human variable regions, for example, by computer modeling, and then comparing the CDR loops and determinants to known human CDR loop structures and determinants. See, e.g., Padlan et al., 1995 FASEB 9:133-39; Chothia et al., 1989 Nature, 342:377-383. Computer modeling may also be used to compare human structural templates selected by sequence homology with the non-human variable regions. See, e.g., Bajorath et al., 1995 Ther. Immunol. 2:95-103; EP-0578515-A3. If humanization of the non-human CDRs results in a decrease in binding affinity, computer modeling may aid in identifying specific amino acid residues that could be changed by site-directed or other mutagenesis techniques to partially, completely or supra-optimally (i.e., increase to a level greater than that of the non-humanized antibody) restore affinity. Those having ordinary skill in the art are familiar with these techniques, and will readily appreciate numerous variations and modifications to such design strategies.

Within certain embodiments, the use of antigen-binding fragments of antibodies may be preferred. Such fragments include Fab fragments or F(ab′)₂ fragments, which may be prepared by proteolytic digestion with papain or pepsin, respectively. The antigen binding fragments may be separated from the Fc fragments by affinity chromatography, for example, using immobilized protein A or protein G, or immobilized DSP-18 polypeptide, or a suitable variant or fragment thereof. Those having ordinary skill in the art can routinely and without undue experimentation determine what is a suitable variant or fragment based on characterization of affinity purified antibodies obtained, for example, using immunodetection methods as provided herein. An alternative method to generate Fab fragments includes mild reduction of F(ab′)₂ fragments followed by alkylation. See, e.g., Weir, Handbook of Experimental Immunology, 1986, Blackwell Scientific, Boston.

According to certain embodiments, non-human, human, or humanized heavy chain and light chain variable regions of any of the above described Ig molecules may be constructed as single chain Fv (sFv) polypeptide fragments (single chain antibodies). See, e.g., Bird et al., 1988 Science 242:423-426; Huston et al., 1988 Proc. Natl. Acad. Sci. USA 85:5879-5883. Multi-functional sFv fusion proteins may be generated by linking a polynucleotide sequence encoding an sFv polypeptide in-frame with at least one polynucleotide sequence encoding any of a variety of known effector proteins. These methods are known in the art, and are disclosed, for example, in EP-B1-0318554, U.S. Pat. Nos. 5,132,405, 5,091,513, and 5,476,786. By way of example, effector proteins may include immunoglobulin constant region sequences. See, e.g., Hollenbaugh et al., 1995 J. Immunol. Methods 188:1-7. Other examples of effector proteins are enzymes. As a non-limiting example, such an enzyme may provide a biological activity for therapeutic purposes (see, e.g., Siemers et al., 1997 Bioconjug. Chem. 8:510-19), or may provide a detectable activity, such as horseradish peroxidase-catalyzed conversion of any of a number of well-known substrates into a detectable product, for diagnostic uses. Still other examples of sFv fusion proteins include Ig-toxin fusions, or immunotoxins, wherein the sFv polypeptide is linked to a toxin. Those having ordinary skill in the art will appreciate that a wide variety of polypeptide sequences have been identified that, under appropriate conditions, are toxic to cells. As used herein, a toxin polypeptide for inclusion in an immunoglobulin-toxin fusion protein may be any polypeptide capable of being introduced to a cell in a manner that compromises cell survival, for example, by directly interfering with a vital function or by inducing apoptosis. Toxins thus may include, for example, ribosome-inactivating proteins, such as Pseudomonas aeruginosa exotoxin A, plant gelonin, bryodin from Bryonia dioica, or the like. See, e.g., Thrush et al., 1996 Annu. Rev. Immunol. 14:49-71; Frankel et al., 1996 Cancer Res. 56:926-32. Numerous other toxins, including chemotherapeutic agents, anti-mitotic agents, antibiotics, inducers of apoptosis (or “apoptogens”, see, e.g., Green and Reed, 1998, Science 281:1309-1312), or the like, are known to those familiar with the art, and the examples provided herein are intended to be illustrative without limiting the scope and spirit of the invention.

The sFv may, in certain embodiments, be fused to peptide or polypeptide domains that permit detection of specific binding between the fusion protein and antigen (e.g., a DSP-18). For example, the fusion polypeptide domain may be an affinity tag polypeptide. Binding of the sFv fusion protein to a binding partner (e.g., a DSP-18) may therefore be detected using an affinity polypeptide or peptide tag, such as an avidin, streptavidin or a His (e.g., polyhistidine) tag, by any of a variety of techniques with which those skilled in the art will be familiar. Detection techniques may also include, for example, binding of an avidin or streptavidin fusion protein to biotin or to a biotin mimetic sequence (see, e.g., Luo et al., 1998 J. Biotechnol. 65:225 and references cited therein), direct covalent modification of a fusion protein with a detectable moiety (e.g., a labeling moiety), non-covalent binding of the fusion protein to a specific labeled reporter molecule, enzymatic modification of a detectable substrate by a fusion protein that includes a portion having enzyme activity, or immobilization (covalent or non-covalent) of the fusion protein on a solid-phase support.

The sFv fusion protein of the present invention, comprising a DSP-18-specific immunoglobulin-derived polypeptide fused to another polypeptide such as an effector peptide having desirable affinity properties, may therefore include, for example, a fusion protein wherein the effector peptide is an enzyme such as glutathione-S-transferase. As another example, sFv fusion proteins may also comprise a DSP-18-specific Ig polypeptide fused to a Staphylococcus aureus protein A polypeptide; protein A encoding nucleic acids and their use in constructing fusion proteins having affinity for immunoglobulin constant regions are disclosed generally, for example, in U.S. Pat. No. 5,100,788. Other useful affinity polypeptides for construction of sFv fusion proteins may include streptavidin fusion proteins, as disclosed, for example, in WO 89/03422; U.S. Pat. Nos. 5,489,528; 5,672,691; WO 93/24631; U.S. Pat. Nos. 5,168,049; 5,272,254 and elsewhere, and avidin fusion proteins (see, e.g., EP 511,747). As provided herein, sFv polypeptide sequences may be fused to fusion polypeptide sequences, including effector protein sequences, that may include full length fusion polypeptides and that may alternatively contain variants or fragments thereof.

An additional method for selecting antibodies that specifically bind to a DSP-18 polypeptide or variant or fragment thereof is by phage display. See, e.g., Winter et al., 1994 Annu. Rev. Immunol. 12:433-55; Burton et al., 1994 Adv. Immunol. 57:191-280. Human or murine immunoglobulin variable region gene combinatorial libraries may be created in phage vectors that can be screened to select Ig fragments (Fab, Fv, sFv, or multimers thereof) that bind specifically to a DSP-18 polypeptide or variant or fragment thereof. See, e.g., U.S. Pat. No. 5,223,409; Huse et al., 1989 Science 246:1275-81; Kang et al., 1991 Proc. Natl. Acad. Sci. USA 88:4363-66; Hoogenboom et al., 1992 J. Molec. Biol. 227:381-388; Schlebusch et al., 1997 Hybridoma 16:47-52 and references cited therein. For example, a library containing a plurality of polynucleotide sequences encoding Ig variable region fragments may be inserted into the genome of a filamentous bacteriophage, such as M13 or a variant thereof, in frame with the sequence encoding a phage coat protein, for instance, gene III or gene VIII of M13, to create an M13 fusion protein. A fusion protein may be a fusion of the coat protein with the light chain variable region domain and/or with the heavy chain variable region domain.

According to certain embodiments, immunoglobulin Fab fragments may also be displayed on the phage particle, as follows. Polynucleotide sequences encoding Ig constant region domains may be inserted into the phage genome in frame with a coat protein. The phage coat fusion protein may thus be fused to an Ig light chain or heavy chain fragment (Fd). For example, from a human Ig library, the polynucleotide sequence encoding the human kappa constant region may be inserted into a vector in frame with the sequence encoding at least one of the phage coat proteins. Additionally or alternatively, the polynucleotide sequence encoding the human IgG1 CH1 domain may be inserted in frame with the sequence encoding at least one other of the phage coat proteins. A plurality of polynucleotide sequences encoding variable region domains (e.g., derived from a DNA library) may then be inserted into the vector in frame with the constant region-coat protein fusions, for expression of Fab fragments fused to a bacteriophage coat protein.

Phage that display an Ig fragment (e.g., an Ig V-region or Fab) that binds to a DSP-18 polypeptide may be selected by mixing the phage library with DSP-18 or a variant or a fragment thereof, or by contacting the phage library with a DSP-18 polypeptide immobilized on a solid matrix under conditions and for a time sufficient to allow binding. Unbound phage are removed by a wash, which typically may be a buffer containing salt (e.g., NaCl) at a low concentration, preferably with less than 100 mM NaCl, more preferably with less than 50 mM NaCl, most preferably with less than 10 mM NaCl, or, alternatively, a buffer containing no salt. Specifically bound phage are then eluted with an NaCl-containing buffer, for example, by increasing the salt concentration in a step-wise manner. Typically, phage that bind the DSP-18 with higher affinity will require higher salt concentrations to be released. Eluted phage may be propagated in an appropriate bacterial host, and generally, successive rounds of DSP-18 binding and elution can be repeated to increase the yield of phage expressing DSP-18-specific immunoglobulin. Combinatorial phage libraries may also be used for humanization of non-human variable regions. See, e.g., Rosok et al., 1996 J. Biol. Chem. 271:22611-18; Rader et al., 1998 Proc. Natl. Acad. Sci. USA 95:8910-15. The DNA sequence of the inserted immunoglobulin gene in the phage so selected may be determined by standard techniques. See, Sambrook et al., 1989 Molecular Cloning: A Laboratory Manual, Cold Spring Harbor Press. The affinity selected Ig-encoding sequence may then be cloned into another suitable vector for expression of the Ig fragment or, optionally, may be cloned into a vector containing Ig constant regions, for expression of whole immunoglobulin chains.

Phage display techniques may also be used to select polypeptides, peptides or single chain antibodies that bind to DSP-18. For examples of suitable vectors having multicloning sites into which candidate nucleic acid molecules (e.g., DNA) encoding such peptides or antibodies may be inserted, see, e.g., MeLafferty et al., Gene 128:29-36, 1993; Scott et al., 1990 Science 249:386-390; Smith et al., 1993 Methods Enzymol. 217:228-257; Fisch et al., 1996, Proc. Natl. Acad. Sci. USA 93:7761-66. The inserted DNA molecules may comprise randomly generated sequences, or may encode variants of a known peptide or polypeptide domain that specifically binds to a DSP-18 polypeptide, or variant or fragment thereof, as provided herein. Generally, the nucleic acid insert encodes a peptide of up to 60 amino acids, more preferably a peptide of 3 to 35 amino acids, and still more preferably a peptide of 6 to 20 amino acids. The peptide encoded by the inserted sequence is displayed on the surface of the bacteriophage. Phage expressing a binding domain for a DSP-18 polypeptide may be selected on the basis of specific binding to an immobilized DSP-18 polypeptide as described above. As provided herein, well-known recombinant genetic techniques may be used to construct fusion proteins containing the fragment thereof. For example, a polypeptide may be generated that comprises a tandem array of two or more similar or dissimilar affinity selected DSP-18 binding peptide domains, in order to maximize binding affinity for DSP-18 of the resulting product.

In certain other embodiments, the invention contemplates DSP-18-specific antibodies that are multimeric antibody fragments. Useful methodologies are described generally, for example in Hayden et al. 1997, Curr Opin. Immunol. 9:201-12; Coloma et al., 1997 Nat. Biotechnol. 15:159-63). For example, multimeric antibody fragments may be created by phage techniques to form miniantibodies (U.S. Pat. No. 5,910,573) or diabodies (Holliger et al., 1997, Cancer Immunol. Immunother. 45:128-130). Multimeric fragments may be generated that are multimers of a DSP-18-specific Fv, or that are bispecific antibodies comprising a DSP-18-specific Fv noncovalently associated with a second Fv having a different antigen specificity. See, e.g., Koelemij et al., 1999 J. Immunother. 22:514-24. As another example, a multimeric antibody may comprise a bispecific antibody having two single chain antibodies or Fab fragments. According to certain related embodiments, a first Ig fragment may be specific for a first antigenic determinant on a DSP-18 polypeptide (or variant or fragment thereof), while a second Ig fragment may be specific for a second antigenic determinant of the DSP-18 polypeptide. Alternatively, in certain other related embodiments, a first immunoglobulin fragment may be specific for an antigenic determinant on a DSP-18 polypeptide or variant or fragment thereof, and a second immunoglobulin fragment may be specific for an antigenic determinant on a second, distinct (i.e., non-DSP-18) molecule. Also contemplated are bispecific antibodies that specifically bind DSP-18, wherein at least one antigen-binding domain is present as a fusion protein.

Introducing amino acid mutations into DSP-18-binding immunoglobulin molecules may be useful to increase the specificity or affinity for DSP-18, or to alter an effector function. Immunoglobulins with higher affinity for DSP-18 may be generated by site-directed mutagenesis of particular residues. Computer assisted three-dimensional molecular modeling may be employed to identify the amino acid residues to be changed, in order to improve affinity for the DSP-18 polypeptide. See, e.g., Mountain et al., 1992, Biotechnol. Genet. Eng. Rev. 10: 1-142. Alternatively, combinatorial libraries of CDRs may be generated in M13 phage and screened for immunoglobulin fragments with improved affinity. See, e.g., Glaser et al., 1992, J. Immunol. 149:3903-3913; Barbas et al., 1994 Proc. Natl. Acad. Sci. USA 91:3809-13; U.S. Pat. No. 5,792,456).

Effector functions may also be altered by site-directed mutagenesis. See, e.g., Duncan et al., 1988 Nature 332:563-64; Morgan et al., 1995 Immunology 86:319-24; Eghtedarzedeh-Kondri et al., 1997 Biotechniques 23:830-34. For example, mutation of the glycosylation site on the Fe portion of the immunoglobulin may alter the ability of the immunoglobulin to fix complement. See, e.g., Wright et al., 1997 Trends Biotechnol. 15:26-32. Other mutations in the constant region domains may alter the ability of the immunoglobulin to fix complement, or to effect antibody-dependent cellular cytotoxicity. See, e.g., Duncan et al., 1988 Nature 332:563-64; Morgan et al., 1995 Immunology 86:319-24; Sensel et al., 1997 Mol. Immunol. 34:1019-29.

The nucleic acid molecules encoding an antibody or fragment thereof that specifically binds DSP-18, as described herein, may be propagated and expressed according to any of a variety of well-known procedures for nucleic acid excision, ligation, transformation and transfection. Thus, in certain embodiments expression of an antibody fragment may be preferred in a prokaryotic host, such as Escherichia coli (see, e.g., Pluckthun et al., 1989 Methods Enzymol. 178:497-515). In certain other embodiments, expression of the antibody or a fragment thereof may be preferred in a eukaryotic host cell, including yeast (e.g., Saccharomyces cerevisiae, Schizosaccharomyces pombe, and Pichia pastoris), animal cells (including mammalian cells) or plant cells. Examples of suitable animal cells include, but are not limited to, myeloma, COS, CHO, or hybridoma cells. Examples of plant cells include tobacco, corn, soybean, and rice cells. By methods known to those having ordinary skill in the art and based on the present disclosure, a nucleic acid vector may be designed for expressing foreign sequences in a particular host system, and then polynucleotide sequences encoding the DSP-18 binding antibody (or fragment thereof) may be inserted. The regulatory elements will vary according to the particular host.

A DSP-18-binding immunoglobulin (or fragment thereof) as described herein may contain a detectable moiety or label such as an enzyme, cytotoxic agent or other reporter molecule, including a dye, radionuclide, luminescent group, fluorescent group, or biotin, or the like. The DSP-18-specific immunoglobulin or fragment thereof may be radiolabeled for diagnostic or therapeutic applications. Techniques for radiolabeling of antibodies are known in the art. See, e.g., Adams 1998 In Vivo 12:11-21; Hiltunen 1993 Acta Oncol. 32:831-9. Therapeutic applications are described in greater detail below and may include use of the DSP-18-binding antibody (or fragment thereof) in conjunction with other therapeutic agents. The antibody or fragment may also be conjugated to a cytotoxic agent as known in the art and provided herein, for example, a toxin, such as a ribosome-inactivating protein, a chemotherapeutic agent, an anti-mitotic agent, an antibiotic or the like.

The invention also contemplates the generation of anti-idiotype antibodies that recognize an antibody (or antigen-binding fragment thereof) that specifically binds to DSP-18 as provided herein, or a variant or fragment thereof. Anti-idiotype antibodies may be generated as polyclonal antibodies or as monoclonal antibodies by the methods described herein, using an anti-DSP-18 antibody (or antigen-binding fragment thereof) as immunogen. Anti-idiotype antibodies or fragments thereof may also be generated by any of the recombinant genetic engineering methods described above, or by phage display selection. An anti-idiotype antibody may react with the antigen binding site of the anti-DSP-18 antibody such that binding of the anti-DSP-18 antibody to a DSP-18 polypeptide is competitively inhibited. Alternatively, an anti-idiotype antibody as provided herein may not competitively inhibit binding of an anti-DSP-18 antibody to a DSP-18 polypeptide.

As provided herein and according to methodologies well known in the art, polyclonal and monoclonal antibodies may be used for the affinity isolation of DSP-18 polypeptides. See, e.g., Hermanson et al., Immobilized Affinity Ligand Techniques, Academic Press, Inc. New York, 1992. Briefly, an antibody (or antigen-binding fragment thereof) may be immobilized on a solid support material, which is then contacted with a sample comprising the polypeptide of interest (e.g., a DSP-18). Following separation from the remainder of the sample, the polypeptide is then released from the immobilized antibody.

### Methods for Detecting DSP-18 Expression

Certain aspects of the present invention provide methods that employ antibodies raised against DSP-18, or hybridizing polynucleotides, for diagnostic and assay purposes. Certain assays involve using an antibody or other agent to detect the presence or absence of DSP-18, or proteolytic fragments thereof. Alternatively, nucleic acid encoding DSP-18 may be detected, using standard hybridization and/or PCR techniques. Suitable probes and primers may be designed by those having ordinary skill in the art based on the DSP-18 cDNA sequences provided herein. Assays may generally be performed using any of a variety of samples obtained from a biological source, such as eukaryotic cells, bacteria, viruses, extracts prepared from such organisms and fluids found within living organisms. Biological samples that may be obtained from a patient include blood samples, biopsy specimens, tissue explants, organ cultures and other tissue or cell preparations. A patient or biological source may be a human or non-human animal, a primary cell culture or culture adapted cell line including but not limited to genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines and the like. In certain preferred embodiments the patient or biological source is a human, and in certain preferred embodiments the biological source is a non-human animal that is a mammal, for example, a rodent (e.g., mouse, rat, hamster, etc.), an ungulate (e.g., bovine) or a non-human primate. In certain other preferred embodiments of the invention, a patient may be suspected of having or being at risk for having a disease associated with altered cellular signal transduction, or may be known to be free of a risk for or presence of such as disease.

To detect DSP-18 protein, the reagent is typically an antibody, which may be prepared as described below. There are a variety of assay formats known to those having ordinary skill in the art for using an antibody to detect a polypeptide in a sample. See, e.g., Harlow and Lane, Antibodies: A Laboratory Manual, Cold Spring Harbor Laboratory, 1988. For example, the assay may be performed in a Western blot format, wherein a protein preparation from the biological sample is resolved by gel electrophoresis, transferred to a suitable membrane and allowed to react with the antibody. The presence of the antibody on the membrane may then be detected using a suitable detection reagent, as described below.

In another embodiment, the assay involves the use of antibody immobilized on a solid support to bind to the target DSP-18 and remove it from the remainder of the sample. The bound DSP-18 may then be detected using a second antibody or reagent that contains a reporter group. Alternatively, a competitive assay may be utilized, in which a DSP-18 polypeptide is labeled with a reporter group and allowed to bind to the immobilized antibody after incubation of the antibody with the sample. The extent to which components of the sample inhibit the binding of the labeled polypeptide to the antibody is indicative of the reactivity of the sample with the immobilized antibody, and as a result, indicative of the level of DSP-18 in the sample.

The solid support may be any material known to those having ordinary skill in the art to which the antibody may be attached, such as a test well in a microtiter plate, a nitrocellulose filter or another suitable membrane. Alternatively, the support may be a bead or disc, such as glass, fiberglass, latex or a plastic such as polystyrene or polyvinylchloride. The antibody may be immobilized on the solid support using a variety of techniques known to those in the art, which are amply described in the patent and scientific literature.

In certain embodiments, the assay for detection of DSP-18 in a sample is a two-antibody sandwich assay. This assay may be performed by first contacting an antibody that has been immobilized on a solid support, commonly the well of a microtiter plate, with the biological sample, such that DSP-18 within the sample is allowed to bind to the immobilized antibody (a 30 minute incubation time at room temperature is generally sufficient). Unbound sample is then removed from the immobilized DSP-18/antibody complexes and a second antibody (containing a reporter group such as an enzyme, dye, radionuclide, luminescent group, fluorescent group or biotin) capable of binding to a different site on the DSP-18 is added. The amount of second antibody that remains bound to the solid support is then determined using a method appropriate for the specific reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products. Standards and standard additions may be used to determine the level of DSP-18 in a sample, using well known techniques.

In a related aspect of the present invention, kits for detecting DSP-18, and for determining DSP-18 phosphatase activity, are provided. Such kits may be designed for detecting the level of DSP-18, or nucleic acid encoding DSP-18, or may detect phosphatase activity of DSP-18 in a direct phosphatase assay or a coupled phosphatase assay. In general, the kits of the present invention comprise one or more containers enclosing elements, such as reagents or buffers, to be used in the assay.

A kit for detecting the level of DSP-18, or nucleic acid encoding DSP-18, typically contains a reagent that binds to the DSP-18 protein, DNA or RNA. To detect nucleic acid encoding DSP-18, the reagent may be a nucleic acid probe or a PCR primer. To detect DSP-18 protein, the reagent is typically an antibody. Such kits also contain a reporter group suitable for direct or indirect detection of the reagent (i.e., the reporter group may be covalently bound to the reagent or may be bound to a second molecule, such as Protein A, Protein G, immunoglobulin or lectin, which is itself capable of binding to the reagent). Suitable reporter groups include, but are not limited to, enzymes (e.g., horseradish peroxidase), substrates, cofactors, inhibitors, dyes, radionuclides, luminescent groups, fluorescent groups and biotin. Such reporter groups may be used to directly or indirectly detect binding of the reagent to a sample component using standard methods known to those having ordinary skill in the art.

Kits for detecting DSP-18 activity typically comprise a DSP-18 substrate in combination with a suitable buffer. DSP-18 activity may be specifically detected by performing an immunoprecipitation step with a DSP-18-specific antibody prior to performing a phosphatase assay as described above. Other reagents for use in detecting dephosphorylation of substrate may also be provided.

Within certain diagnostic assays, a proliferative disorder may be detected in a patient or another biological source organism as provided herein, based on the presence of an altered DSP-18 or an altered level of DSP-18 expression. For example, an antibody may distinguish between a wild-type DSP-18 and an altered DSP-18 having a variation in amino acid sequence. Such a variation may be indicative of the presence of a proliferative disorder, or of susceptibility to such a disorder. Hybridization and amplification techniques may be similarly used to detect modified DSP-1 8 sequences.

Methods for Identifying Modulators of DSP-18 Activity

In one aspect of the present invention, DSP-18 polypeptides may be used to identify agents that modulate DSP-18 activity. Such agents may inhibit or enhance signal transduction via a MAP-kinase cascade, leading to cell proliferation. An agent that modulates DSP-18 activity may alter expression and/or stability of DSP-18, DSP-18 protein activity and/or the ability of DSP-18 to dephosphorylate a substrate. Agents that may be screened within such assays include, but are not limited to, antibodies and antigen-binding fragments thereof, competing substrates or peptides that represent, for example, a catalytic site or a dual phosphorylation motif, antisense polynucleotides and ribozymes that interfere with transcription and/or translation of DSP-18 and other natural and synthetic molecules, for example small molecule inhibitors, that bind to and inactivate DSP-18.

Candidate agents for use in a method of screening for a modulator of DSP-18 according to the present invention may be provided as “libraries” or collections of compounds, compositions or molecules. Such molecules typically include compounds known in the art as “small molecules” and having molecular weights less than 10⁵ daltons, preferably less than 10⁴ daltons and still more preferably less than 10³ daltons. For example, members of a library of test compounds can be administered to a plurality of samples, each containing at least one DSP-18 polypeptide as provided herein, and then assayed for their ability to enhance or inhibit DSP-18-mediated dephosphorylation of, or binding to, a substrate. Compounds so identified as capable of influencing DSP-18 function (e.g., phosphotyrosine and/or phosphoserine/threonine dephosphorylation) are valuable for therapeutic and/or diagnostic purposes, since they permit treatment and/or detection of diseases associated with DSP-18 activity. Such compounds are also valuable in research directed to molecular signaling mechanisms that involve DSP-18, and to refinements in the discovery and development of future DSP-18 compounds exhibiting greater specificity.

Candidate agents further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694, PCT/US91/04666, which are hereby incorporated by reference in their entireties) or other compositions that may include small molecules as provided herein (see e.g., PCT/US94/08542, EP 0774464, U.S. Pat. Nos. 5,798,035, 5,789,172, 5,751,629, which are hereby incorporated by reference in their entireties). Those having ordinary skill in the art will appreciate that a diverse assortment of such libraries may be prepared according to established procedures, and tested using DSP-18 according to the present disclosure.

In certain embodiments, modulating agents may be identified by combining a candidate agent with a DSP-18 polypeptide or a polynucleotide encoding such a polypeptide, in vitro or in vivo, and evaluating the effect of the candidate agent on the DSP-18 phosphatase activity using, for example, a representative assay described herein. An increase or decrease in phosphatase activity can be measured by performing a representative assay provided herein in the presence and absence of a candidate agent. Briefly, a candidate agent may be included in a mixture of active DSP-18 polypeptide and substrate (e.g., a phosphorylated MAP-kinase), with or without pre-incubation with one or more components of the mixture. In general, a suitable amount of antibody or other agent for use in such an assay ranges from about 0.01 μM to about 100 μM. The effect of the agent on DSP-18 activity may then be evaluated by quantifying the loss of phosphate from the substrate, and comparing the loss with that achieved using DSP-18 without the addition of a candidate agent. Alternatively, a coupled kinase assay may be used, in which DSP-18 activity is indirectly measured based on MAP-kinase activity.

Alternatively, a polynucleotide comprising a DSP-18 promoter, operably linked to a DSP-18 coding region or reporter gene may be used to evaluate the effect of a test compound on DSP-18 transcription. Such assays may be performed in cells that express DSP-18 endogenously (e.g., human or other mammalian skeletal muscle cells) or in cells transfected with an expression vector comprising a DSP-18 promoter, linked to a reporter gene. The effect of a test compound may then be evaluated by assaying the effect on transcription of DSP-18 or the reporter using, for example, a Northern blot analysis or a suitable reporter activity assay.

DSP-18 activity may also be measured in whole cells transfected with a reporter gene whose expression is dependent upon the activation of an appropriate substrate. For example, appropriate cells (i.e., cells that express DSP-18) may be transfected with a substrate-dependent promoter linked to a reporter gene. In such a system, expression of the reporter gene (which may be readily detected using methods well known to those of ordinary skill in the art) depends upon activation of substrate. Dephosphorylation of substrate may be detected based on a decrease in reporter activity. Candidate modulating agents may be added to such a system, as described above, to evaluate their effect on DSP-18 activity.

The present invention further provides methods for identifying a molecule that interacts with, or binds to, DSP-18. Such a molecule generally associates with DSP-18 with an affinity constant (Kₐ) of at least 10^{4,} preferably at least 10⁵, more preferably at least 10⁶, still more preferably at least 10⁷ and most preferably at least 10⁸. Affinity constants may be determined using well known techniques. Methods for identifying interacting molecules may be used, for example, as initial screens for modulating agents, or to identify factors that are involved in the in vivo DSP-18 activity. Techniques for substrate trapping, for example using variants or substrate trapping mutants of DSP-18 as described above, are also contemplated according to certain embodiments provided herein. In addition to standard binding assays, there are many other techniques that are well known for identifying interacting molecules, including yeast two-hybrid screens, phage display and affinity techniques. Such techniques may be performed using routine protocols, which are well known to those having ordinary skill in the art (see, e.g., Bartel et al., In Cellular Interactions in Development: A Practical Approach, D. A. Harley, ed., Oxford University Press (Oxford, UK), pp. 153-179, 1993). Within these and other techniques, candidate interacting proteins (e.g., putative DSP-18 substrates) may be phosphorylated prior to assaying for interacting proteins.

Within other aspects, the present invention provides animal models in which an animal either does not express a functional DSP-18, or expresses an altered DSP-18. Such animals may be generated using standard homologous recombination strategies. Animal models generated in this manner may be used to study activities of DSP-18 polypeptides and modulating agents in vivo.

Methods for Dephosphorylating a Substrate

In another aspect of the present invention, a DSP-18 polypeptide may be used for dephosphorylating a substrate of DSP-18 as provided herein. In one embodiment, a substrate may be dephosphorylated in vitro by incubating a DSP-18 polypeptide with a substrate in a suitable buffer (e.g., Tris, pH 7.5, 1 mM EDTA, 1 mM dithiothreitol, 1 mg/mL bovine serum albumin) for 10 minutes at 30° C. Any compound that can be dephosphorylated by DSP-18, such as a MAP-kinase, may be used as a substrate. In general, the amounts of the reaction components may range from about 50 pg to about 50 ng of DSP-18 polypeptide and from about 10 ng to about 10 μg of substrate. Dephosphorylated substrate may then be purified, for example, by affinity techniques and/or gel electrophoresis. The extent of substrate dephosphorylation may generally be monitored by adding [γ-³²P]-labeled substrate to a test aliquot, and evaluating the level of substrate dephosphorylation as described herein.

Methods for Modulating Cellular Responses

Modulating agents may be used to modulate, modify or otherwise alter (e.g., increase or decrease) cellular responses, such as cell proliferation, differentiation and survival, in a variety of contexts in vivo and in vitro. In general, to so modulate (e.g., increase or decrease in a statistically significant manner) such a response, a cell is contacted with an agent that modulates DSP-18 activity, under conditions and for a time sufficient to permit modulation of DSP-18 activity. Agents that modulate a cellular response may function in any of a variety of ways. For example, an agent may modulate a pattern of gene expression (i.e., may enhance or inhibit expression of a family of genes or genes that are expressed in a coordinated fashion). A variety of hybridization and amplification techniques are available for evaluating patterns of gene expression. Alternatively, or in addition, an agent may effect apoptosis or necrosis of the cell, and/or may modulate the functioning of the cell cycle within the cell. (See, e.g., Ashkenazi et al., 1998 Science, 281:1305; Thomberry et al., 1998 Science 281:1312; Evan et al., 1998 Science 281:1317; Adams et al., 1998 Science 281:1322; and references cited therein.)

Cells treated as described above may exhibit standard characteristics of cells having altered proliferation, differentiation or survival properties. In addition, such cells may (but need not) display alterations in other detectable properties, such as contact inhibition of cell growth, anchorage independent growth or altered intercellular adhesion. Such properties may be readily detected using techniques with which those having ordinary skill in the art will be familiar.

Therapeutic Methods

One or more DSP-18 polypeptides, modulating agents (including any agent that specifically binds a DSP-18, such as an antibody or fragment thereof as provided herein) and/or polynucleotides encoding such polypeptides and/or modulating agents may also be used to modulate DSP-18 activity in a patient. As used herein, a “patient” may be any mammal, including a human, and may be afflicted with a condition associated with DSP-18 activity or may be free of detectable disease. Accordingly, the treatment may be of an existing disease or may be prophylactic. Conditions associated with DSP-18 activity include any disorder associated with cell proliferation, including Duchenne muscular dystrophy, cancer, graft-versus-host disease (GVHD), autoimmune diseases, allergy or other conditions in which immunosuppression may be involved, metabolic diseases, abnormal cell growth or proliferation and cell cycle abnormalities. Certain such disorders involve loss of normal MAP-kinase phosphatase activity, leading to uncontrolled cell growth. DSP-18 polypeptides, and polynucleotides encoding such polypeptides, can be used to ameliorate such disorders.

For administration to a patient, one or more polypeptides, polynucleotides and/or modulating agents are generally formulated as a pharmaceutical composition. A pharmaceutical composition may be a sterile aqueous or non-aqueous solution, suspension or emulsion, which additionally comprises a physiologically acceptable carrier (i.e., a non-toxic material that does not interfere with the activity of the active ingredient). Such compositions may be in the form of a solid, liquid or gas (aerosol). Alternatively, compositions of the present invention may be formulated as a lyophilizate or compounds may be encapsulated within liposomes using well known technology. Pharmaceutical compositions within the scope of the present invention may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents and/or preservatives.

Any suitable carrier known to those of ordinary skill in the art may be employed in the pharmaceutical compositions of the present invention. Carriers for therapeutic use are well known, and are described, for example, in Remingtons Pharmaceutical Sciences, Mack Publishing Co. (A. R. Gennaro ed. 1985). In general, the type of carrier is selected based on the mode of administration. Pharmaceutical compositions may be formulated for any appropriate manner of administration, including, for example, topical, oral, nasal, intrathecal, rectal, vaginal, sublingual or parenteral administration, including subcutaneous, intravenous, intramuscular, intrasternal, intracavernous, intrameatal or intraurethral injection or infusion. For parenteral administration, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

A pharmaceutical composition (e.g., for oral administration or delivery by injection) may be in the form of a liquid (e.g., an elixir, syrup, solution, emulsion or suspension). A liquid pharmaceutical composition may include, for example, one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile.

The compositions described herein may be formulated for sustained release (i.e., a formulation such as a capsule or sponge that effects a slow release of compound following administration). Such compositions may generally be prepared using well known technology and administered by, for example, oral, rectal or subcutaneous implantation, or by implantation at the desired target site. Sustained-release formulations may contain an agent dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

For pharmaceutical compositions comprising a polynucleotide encoding a DSP-18 polypeptide and/or modulating agent (such that the polypeptide and/or modulating agent is generated in situ), the polynucleotide may be present within any of a variety of delivery systems known to those of ordinary skill in the art, including nucleic acid, and bacterial, viral and mammalian expression systems. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

Within a pharmaceutical composition, a DSP-18 polypeptide, polynucleotide or modulating agent may be linked to any of a variety of compounds. For example, such an agent may be linked to a targeting moiety (e.g., a monoclonal or polyclonal antibody, a protein or a liposome) that facilitates the delivery of the agent to the target site. As used herein, a “targeting moiety” may be any substance (such as a compound or cell) that, when linked to an agent enhances the transport of the agent to a target cell or tissue, thereby increasing the local concentration of the agent. Targeting moieties include antibodies or fragments thereof, receptors, ligands and other molecules that bind to cells of, or in the vicinity of, the target tissue. An antibody targeting agent may be an intact (whole) molecule, a fragment thereof, or a functional equivalent thereof. Examples of antibody fragments are F(ab′)₂, -Fab′, Fab and F[v] fragments, which may be produced by conventional methods or by genetic or protein engineering. Linkage is generally covalent and may be achieved by, for example, direct condensation or other reactions, or by way of bi- or multi-functional linkers. Targeting moieties may be selected based on the cell(s) or tissue(s) toward which the agent is expected to exert a therapeutic benefit.

Pharmaceutical compositions may be administered in a manner appropriate to the disease to be treated (or prevented). An appropriate dosage and a suitable duration and frequency of administration will be determined by such factors as the condition of the patient, the type and severity of the patient's disease, the particular form of the active ingredient and the method of administration. In general, an appropriate dosage and treatment regimen provides the agent(s) in an amount sufficient to provide therapeutic and/or prophylactic benefit (e.g., an improved clinical outcome, such as more frequent complete or partial remissions, or longer disease-free and/or overall survival). For prophylactic use, a dose should be sufficient to prevent, delay the onset of or diminish the severity of a disease associated with cell proliferation.

Optimal dosages may generally be determined using experimental models and/or clinical trials. In general, the amount of polypeptide present in a dose, or produced in situ by DNA present in a dose, ranges from about 0.01 μg to about 100 μg per kg of host, typically from about 0.1 μg to about 10 μg. The use of the minimum dosage that is sufficient to provide effective therapy is usually preferred. Patients may generally be monitored for therapeutic or prophylactic effectiveness using assays suitable for the condition being treated or prevented, which will be familiar to those having ordinary skill in the art. Suitable dose sizes will vary with the size of the patient, but will typically range from about 10 mL to about 500 mL for 10-60 kg animal.

The following Examples are offered by way of illustration and not by way of limitation.

### EXAMPLES

### Example 1

### Cloning and Sequencing cDNA Encoding DSP-18

This Example illustrates the cloning of a cDNA molecule encoding a human prototype DSP-18.

A conserved sequence motif surrounding the active site domain of dual-specificity phosphatases was identified as follows: Dual specificity phosphatases belong to the larger family of protein tyrosine phosphatases (PTPs) that share a conserved catalytic domain containing a cysteine residue situated N-terminal to a stretch of five variable amino acids followed by an arginine residue (Fauman et al., Trends In Bioch. Sci. 21:413-417, 1996). DSPs typically contain a PTP active site motif but lack sequence homology to PTPs in other regions (Jia, Biochem. and Cell Biol. 75:17-26, 1997). An alignment of eight amino acid sequences derived from eight human DSPs having MAP-kinase phosphatase activity yielded a conserved homology region consisting of a 24-amino acid peptide sequence containing the PTP active site signature motif. Thus, a candidate peptide having the sequence:

| | |
|---|---|
| NGRVLVHCQAGISRSGTNILAYLM | SEQ ID NO:16 |

was used to search the GenBank “month” database (Natl. Center for Biol. Information, >http://www.ncbi.nlm.nih.gov/Genbank/>) for nucleotide sequences potentially capable of encoding identical or similar PTP active site sequences. The search employed an algorithm (tblastn) capable of reverse translation of the candidate peptide with iterations allowing for genetic code degeneracy within default parameters.

The search results identified a human genomic DNA sequence, GenBank Accession No. AL160175, which has been assigned to a region of human chromosome 20. The GenBank record for AL160175 disclosed no open reading frame encoding a phosphatase, nor were exon/intron boundaries defined in a manner permitting the determination of an open reading frame encoding a DSP active site domain. It was also not clear whether the region of the genomic AL160175 sequence corresponding to the reverse translated query sequence (SEQ ID NO:16) represented an exonic or intronic DNA sequence, hence the query sequence was resubmitted to the “month,” “nr,” and dbEST databases to identify evidence for an identical or highly similar sequence that was an expressed sequence.

The search identified two highly homologous expressed sequence tag (EST) database entries, GenBank Accession Nos. BF377364 and BF377396, each containing a sequence region capable of encoding a dual specificity phosphatase catalytic domain containing a polypeptide sequence with high homology to SEQ ID NO:16. Alignment of these two ESTs provided a consensus sequence that was used as a query sequence for a tblastn search, which was performed as described above except the GenBank “nr” database was queried. The search results retrieved a murine EST (Accession No. BE653350), which showed high homology with the BF377364/BF377396 consensus sequence in a large region of overlap, and which also contained an additional sequence located 5′ to the overlap. The BE653350 sequence was then aligned with the human genomic sequence AL160175 described above, in order to identify sequence segments exhibiting high homology, from which to estimate the positions of exon/intron boundaries in the human genomic sequence. A partial, presumptively spliced BF377396 coding sequence was thus constructed and its translated amino acid sequence deduced. This amino acid sequence exhibited high homology when aligned with, but still lacked the N-terminal region of, the amino acid sequence of a related polypeptide, DSP-3 (SEQ ID NO:15), which is disclosed in U.S. application Ser. No. 09/608,062. Reverse translation and alignment of the DSP-3 exons with the genomic AL160175 sequence identified an extended hypothetical new dual specificity phosphatase coding sequence. The resulting prototypical DSP-18 (DSP-18pr) polynucleotide (SEQ ID NO:13) and polypeptide (SEQ ID NO:14) sequences are shown in FIG. 1, wherein nucleotide start and stop codons and theoretical RNA splice donor and acceptor sites are highlighted in boldface type. When the prototypical cDNA fragment encoding DSP-18 (SEQ ID NO:13) was submitted as a search query to SwissProt and Genbank, no sequence matches were identified.

Regions of the DSP-18pr cDNA sequence (SEQ ID NO:13) located 5′ to and 3′ to the catalytic domain were used to design the following oligonucleotide primers for PCR.

DSP18-FL-5′EcoRI:

| | |
|---|---|
| 5′-GGAATTCAAGGCAATGGCATGACCAAGGTACTTC | SEQ ID NO: 18 |

CTGGA-3′

DSP18-FL-3′EcoRI:

| | |
|---|---|
| 5′-GGAATTCACTTGCCGCCCTTGCGGGA-3′ | SEQ ID NO:19 |

Standard molecular biology procedures and methods were used, including nucleic acid preparation, PCR amplification, nucleic acid sequencing, etc. Plasmid isolation, production of competent cells, transformation and plasmid manipulations were carried out according to published procedures (Sambrook et al., Molecular Cloning, A Laboratory Manual, Cold Spring Harbor Laboratory Press, Cold Spring Harbor, N.Y. (1989); Ausubel et al., Current Protocols in Molecular Biology, Greene Publishing & John Wiley & Sons, NY (1994)). The DSP18-FL-5′EcoRI and DSP18-FL-3′ExoRI primers were used in standard PCR amplification reactions with human testis cDNA (Marathon™-ready cDNA, #7414-1, Clontech, Palo Alto, Calif.) as template, and amplification reaction products were resolved by agarose gel electrophoresis.

An amplicon of 700 bp was obtained upon secondary amplification using the DSP18-FL-5′EcoRI and DSP18-FL-3′ExoRI primers (following a primary amplification using the same primers with the primers DSP18 5'short and DSP18 3'short, described below, to generate the templates for secondary amplification), excised from the gel and sequenced. The resulting DSP-18 encoding sequence was designated prototypical DSP-18 (DSP-18pr) (FIG. 1, SEQ ID NO:13). The DSP18pr amplicon was ligated into a modified bacterial pGEX-6PKG expression vector (, Amersham Biosciences, Piscataway, N.J.), referred to as pGEX-6P1, according to standard methods known in the molecular biology art. Bacterial (E. coli) transformation, IPTG induction of recombinant protein expression, bacterial cell lysis, affinity isolation of the GST fusion protein on solid-phase immobilized glutathione (Amersham), and enterokinase (PreScission™ Protease, Amersham Biosciences) cleavage of the fusion protein to liberate expressed recombinant DSP-18pr were all performed according to standard procedures provided by the vector supplier.

High levels of DSP-18pr expression were detected, and the expressed recombinant protein was assayed for phosphatase catalytic activity using 6,8-difluoro-4-methylumbelliferyl phosphate (DiFMUP, Molecular Probes, Inc., Eugene, Oreg.) as a substrate. DSP-18pr was diluted serially in assay buffer (25 mM Tris, pH7.5; 1 mM EDTA, 0.3 mg/ml ovalbumin, and 1 mM DTT). After the addition of 6 μl of diluted enzyme to 54 μl of 1 μM substrate and incubation for 15 min., the assay mixture was measured for fluorescence intensity at 400 nm wavelength in an LJL TriLux™ fluorescent plate reader (LJL BioSystems, Inc., Sunnyvale, Calif.) according to the manufacturer's recommendations. Detectable catalytic dephosphorylation of substrate was observed only in the presence of the recombinant enzyme, and occurred in an enzyme concentration and time-dependent manner.

To determine if the DSP-18pr encoding cDNA sequence was a full-length sequence, human testis cDNA was screened in 5′ and 3′ RACE (rapid amplification of cDNA ends) reactions as described (Frohman et al., Proc. Natl. Acad. Sci. USA 85:8998, 1988; Ohara et al., Proc. Natl. Acad. Sci. USA 86:5673, 1989; Loh et al., Science 243:217, 1989) using 5′/3′ RACE kits (Boehringer Mannheim, Indianapolis, Ind.; Clontech, Palo Alto, Calif.; Life Technologies, Inc., Gaithersburg, Md.) according to the supplier's instructions, and with the following primers (SEQ ID NOs:20-23) to determine 5′ and 3′ sequences. The 5′ RACE primers were as follows.

DSP18 5′short:

| | |
|---|---|
| 5′-GCATCCCGGTCGCTGATACCCCTGA-3′ | SEQ ID NO:20 |
| DSP18 Race-dn1: | |
| 5′-GCTAGGCTGGCGGGACGTGCTTGA-3′ | SEQ ID NO:21 |

The 3′ RACE primers were as follows:

| | |
|---|---|
| DSP18 Race-up: | |
| 5′-TCAGGGGTATCAGCGACCGGGATGC-3′ | SEQ ID NO:22 |
| DSP18 3′short | |
| 5′-TCAAGCACGTCCCGCCAGCCTAGC-3′ | SEQ ID NO:23 |

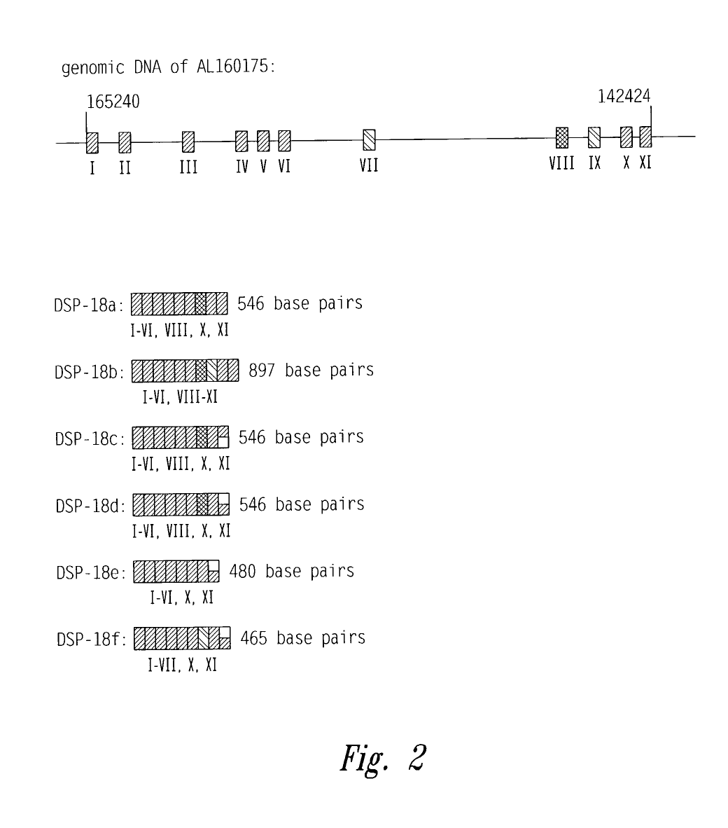

The results of RACE analyses revealed the presence of several distinct DSP-18 splice variants that apparently encoded distinct DSP-18 isoforms. As shown in FIGS. 2-6, RACE products corresponding to transcripts encoding at least six distinct DSP-18 isoforms (DSP-18a-f) could be identified according to a scheme that employed common utilization of exons I-VI, X, and XI by all isoforms, and differential utilization of exons VII-IX among the different isoforms (FIG. 2). Alignment of the amino acid sequences of DSP-18a (SEQ ID NO:2) and DSP-3 (SEQ ID NO:15) is shown in FIG. 7.

### Example 2

DSP-18 Expression in Human Tissues

In this example, a DSP-18 encoding nucleic acid sequence is shown to hybridize to human polyA+RNA from various tissue sources. A DSP-18-specific DNA probe was prepared by PCR amplification of the DSP-18 encoding cDNA (SEQ ID NO:13) using the prototypical DSP-18 (DSP-18pr) encoding plasmid described in Example 1 as template, with the following primers.

| HuDSP18-5═NB: | |
|---|---|
| 5′-CAC CCC AGC CTC TGC TGC AGG ATAT | (SEQ ID NO:24) |
| CAC C-3′ | |
| HuDSP18-3′NB: | |
| 5′-CTG GCC GAG CCA AAC TCT TCA AGC | (SEQ ID NO:25) |
| TGT G-3′ | |

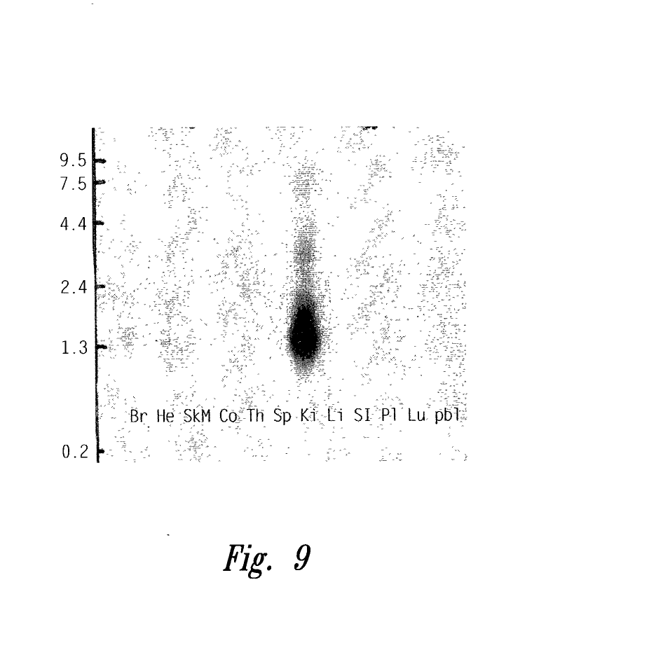

The amplicon was gel-purified and ³²P-labeled by the random primer method as described in Ausubel et al. (Current Protocols in Molecular Biology, Greene Publ. Assoc. Inc. & John Wiley & Sons, Inc., Boston, Mass. (1998)) for use as a nucleic acid hybridization probe. The probe was hybridized to blots containing human polyA+RNA derived from multiple human tissues, normalized for the amount of detectable β-actin mRNA (FIG. 9, Clontech, Inc., Palo Alto, Calif.). Blots underwent prehybridization for 30 min at 68° C. in Express Hyb™ solution (Clontech), and then were hybridized with the labeled probe for 1 hour at 68° C. in Express Hyb™ solution. The blots were next washed for 40 min at room temperature in 2×SSC, 0.05% SDS, followed by a second wash for 40 min at 50° C. in 0.1×SSC, 0.1% SDS. Blots were air-dried and then exposed to Hyper film MP™ autoradiographic film (Amersham Life Sciences, Arlington Hts, Ill.) overnight. Results are shown in FIG. 9, in which the human tissue sources for the RNAs were as follows: Br, brain; He, heart; SkM, skeletal muscle; Co, colon; Th, thymus; Sp, spleen; Ki, kidney; Li, liver; SI, small intestine; Pl, placenta; Lu, lung; pbl, peripheral blood lymphocytes.

To independently assess DSP-18 expression in various human tissues, real time PCR analysis was performed with the GeneAmp™ 5700 (Applied Biosystems Division, Perkin-Elmer Corp., Foster City, Calif.) according to the manufacturer's instructions, and using cDNA templates from a variety of cell and tissue type-specific cDNA libraries (human brain, heart, skeletal muscle, thymus, kidney, liver, lung and testis; all from Clontech, Inc., Palo Alto, Calif.) with the following primers.

| | |
|---|---|
| hDSP18-396F: | |
| 5′-GCA GCA GCT TGA AGA GTT TGG-3′ | (SEQ ID NO:26) |
| hDSP18-472R: | |
| 5′-GGG CAC CAG AGG TTT TTG AG-3′ | (SEQ ID NO:27) |
| TaqMan™ probe: hDSP18-481-rev-T: | |
| 5′-CCT ATG TCT GGC ACC CTT CTG GGA | (SEQ ID NO:28) |

ACT G-3′

In addition to the DSP-18 expression detected in human tissues by northern blot analysis as described above, DSP-18 expression was detected in human testis using real time PCR.

### Example 3

### Substrate Trapping Mutant DSP-18 Constructs

A recombinant expression construct was prepared that encodes a substrate trapping mutant DSP-18 differing from prototypical DSP-18 (DSP-18pr, SEQ ID NO:14) by having the cysteine residue at amino acid position 103 replaced with a serine, encoding the mutant DSP-18pr (C103S). Oligonucleotide-directed, site-specific mutagenesis was employed to modify the DSP-18pr expression construct described in Example 1, according to the method of Kunkel et al. (Methods in Enzymol. 154:367 (1987)). The following oligonucleotide primers were used.

DSP18-CtoS sense:

| 5′-GGAACTGCCTTGTGCACTCCTTTGCAGGCATCT | (SEQ ID NO:29) |
|---|---|
| CTCGC-3′ | |

DSP18-CtoS antisense:

| 5′-GCGAGAGATGCCTGCAAAGGAGTGCACAAGGCA | (SEQ ID NO:30) |
|---|---|
| GTTCC-3′ | |

A second recombinant expression construct encoded a second substrate trapping mutant DSP-18pr that differed from DSP-18pr (SEQ ID NO:14) in that the aspartate residue at position 72 was replaced with an alanine residue, providing the mutant referred to as DSP-18pr D72A. The DSP-18pr D72A expression construct was prepared by Retrogen (San Diego, Calif.), according to the vendor's protocol.

Vectors for expression of DSP-18pr wild type (WT), DSP-18pr C103S, and DSP-18pr D72A were prepared as follows. Vector pCMVTag2B (Stratagene, La Jolla, Calif.) was digested with restriction endonuclease BamHI (New England Biolabs, Beverly, MA) for 3 hours at 37° C. The digested vector was then incubated with Klenow polymerase (New England Biolabs) for 15 minutes at 25° C. to fill in the recessed 3′ termini, followed by an incubation of 30 minutes at 37° C. with calf intestinal phosphatase (New England Biolabs). The GATEWAY™ Reading Frame Cassette B (Invitrogen, Carlsbad, Calif.) was inserted into the pCMVTag2B vector by ligation with T4 DNA ligase (Invitrogen) overnight at 16° C. according to the supplier's instructions. DB3.1 ™ competent E. coli cells were transformed with the ligated vector (GWpCMVTag2) and DNA was isolated by standard molecular biology methods. The DSP-18pr WT construct prepared as described in Example 1, DSP-18pr C103S and DSP-18pr D72A constructs, and the pENTR™ 1A entry vector (Invitrogen) were digested with EcoRI (New England Biolabs) for 3 hours at 37° C. The pENTR™ 1A clone was treated with calf intestinal phosphatase for 30 minutes at 37 ° C., and then the DSP-18 WT and the substrate trapping mutant constructs were inserted into pENTR™ by ligation with T4 DNA ligase overnight at 16° C. Vector DNA was prepared from LIBRARY EFFICIENCY® DH5α™ cells (Invitrogen) transformed with each construct according to the supplier's recommendation.

FLAG® epitope-tagged DSP-18pr WT, DSP-18pr C103S, and DSP-18pr D72A polypeptides were prepared by cloning the pENTR™ 1A-DSP-18 WT and substrate trapping mutant constructs into the GWpCMVTag2 vector. The pENTR™ 1A constructs containing each of the DSP-18 polynucleotides were linearized by digesting the constructs with Vsp 1 (Promega Corp., Madison, Wis.) for 2 hours at 37° C. for 2 hours. The DNA was purified using a QIAGEN PCR Purification kit (QIAGEN, Inc., Valencia, Calif.), and 30 μl (100 ng/μl) was combined in a GATEWAY™ LR reaction with 6 μl linearized pENTR™ 1A-DSP-18 WT, pENTR™ 1A-DSP-18pr D72A, or pENTR™ 1A-DSP-18pr C103S, 3 μl TE buffer, 4 μl Clonase™ Enzyme, and 4 μl LR reaction buffer (Invitrogen) for 1 hour at room temperature. After addition of Proteinase K (Invitrogen) to each reaction for 10 minutes, LIBRARY EFFICIENCY® DH5α™ were transformed with each expression vector. 293-HEK cells, maintained in DMEM, 10% (v/v) fetal bovine serum (FBS) at 37° C. and 5% CO₂, were transfected with the expression vectors or vector alone using the LipofectAMINE reagent (Invitrogen) according to the manufacturer's instructions.

### Example 4

### Phosphatase Activity of DSP-18 and DSP-18 Substrate Trapping Mutants

This example compares the enzyme activity of prototypical DSP-18 wild-type (DSP-18pr WT) with the activity of the substrate trapping mutants, DSP-18pr C103S and DSP-18pr D72A.

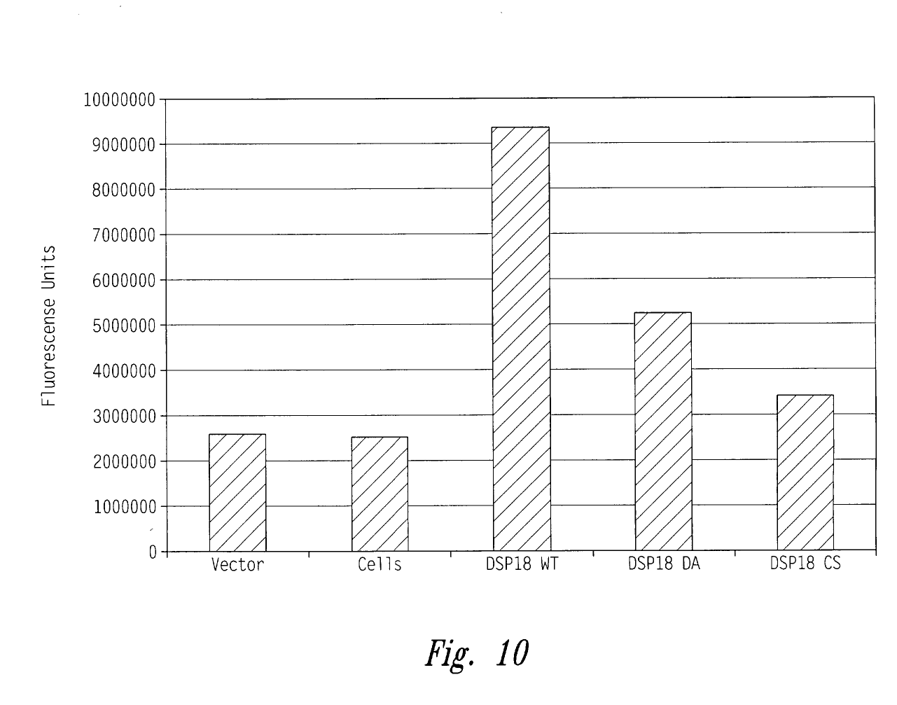

DSP-18pr WT and the substrate trapping mutants expressed in 293HEK cells were isolated by immunoprecipitation (IP); untransfected cells, and cells transfected wit empty vector were also analyzed as controls. Twenty-four hours after transfection, the cells were harvested and lysed in IP buffer (25 mM Tris 7.2, 150 mM NaCl, 1 mM EGTA, 1 mM EDTA, 2 mM DTT, 1% Triton, with Complete Protease Inhibitor (Roche Applied Bioscience, Indianapolis, Ind.). The cell extracts (150 μl per sample) were combined with 10 μl anti-FLAG® M2 agarose beads (Sigma-Aldrich Co., St. Louis, Mo.) and incubated for 60 minutes at 4° C. The agarose beads were separated from the cell extracts by centrifugation and then resuspended in 100 μl IP buffer. Two 25 μl aliquots of the resuspended beads were each incubated with 25 μl of substrate solution (1 μM DifMUP, Molecular Probes, Inc.) at room temperature for one hour. The agarose beads were then removed by centrifugation, and the supernatant was collected. Equal volumes (40 μl) of supernatant and water were combined to terminate the reaction. Catalytic dephosphorylation of substrate was measured by determining fluorescence intensity at 400 nm (see Example 1). Results are shown in FIG. 10.

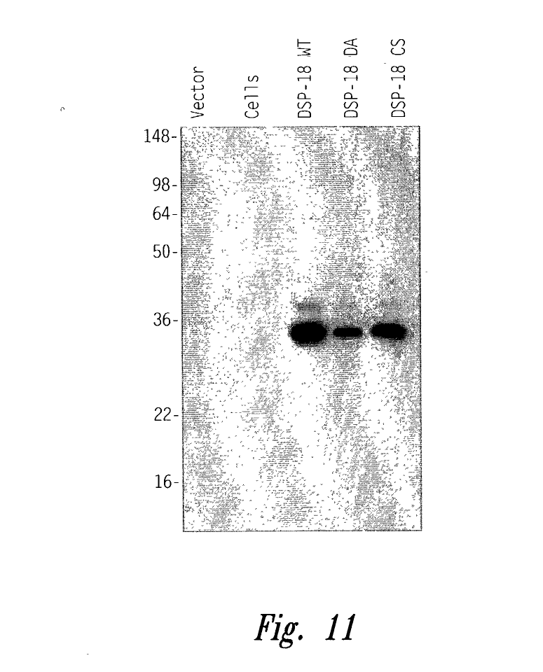

To show that the difference in phosphatase activity between the sample containing DSP-18pr WT and the samples containing the DSP-18pr substrate trapping mutants was not related to differences in expression levels of the polypeptides, cell extracts were analyzed by immunoblot. The agarose beads recovered from the DiFMUP activity assay (above) were washed with 1 ml TBS (25 mM Tris 7.5, 150 mM NaCl), and the duplicate samples of beads were combined. The beads were resuspended in 30 μl of TBS containing 150 μg/ml 3X-FLAG® peptide (Sigma-Aldrich) to competitively release bound FLAG® fusion proteins, and incubated at 4° C., overnight with gentle rocking. After incubation, the samples were centrifuged to pellet the beads, and 10 μl of each supernatant were combined with 10 μl of SDS-PAGE reducing sample buffer. The samples were heated at 95° C. for five minutes, and then applied to a 14% Tris-glycine SDS-PAGE gel (NOVEX® from Invitrogen Life Technologies, Carlsbad, Calif.). After electrophoresis, the separated proteins were electrophorectically transferred from the gel onto an Immobilon-P polyvinylidene fluoride (PVDF) membrane (Millipore, Bedford, Mass.). The PVDF membrane was blocked in 5% milk in TBST (20 mM Tris pH 7.5, 150 mM NaCl, 0.05% Tween-20), incubated with an anti-FLAG® M2 antibody (1:4000) (Sigma-Aldrich) for 1 hour at room temperature, washed 3×10 minutes with TBST, and then incubated with horseradish peroxidase (HRP) conjugated goat anti-mouse IgG (1:10,000) (Amersham Biosciences, Piscataway, N.J.) for 30 minutes at room temperature. Binding was detected with the Western Lightning Chemiluminescent reagent used according to the manufacturer's instructions (Perkin-Elmer Life Sciences, Boston, Mass.) as shown in FIG. 11.

### Example 5

### Dephosphorylation of Different Substrates by DSP-18

This example compares dephosphorylation of different substrates by DSP-18 and by another dual specificity phosphatase, DSP-3.

Bacterial cells (E. coli, strain BL-21) were transformed with either a recombinant DSP 18pr expression construct (pGEX-6PKG) or with a recombinant expression construct containing a polynucleotide sequence [SEQ ID NO:34] encoding DSP3 polypeptide [SEQ ID NO:15]. The cells were induced to produce recombinant protein by the addition of 100 μM IPTG. Bacterial cell lysis, affinity isolation of the GST fusion protein on solid-phase immobilized glutathione (Amersham-Pharmacia), and PreScission Protease (Amersham-Pharmacia) cleavage of the fusion protein to liberate expressed recombinant DSP-18pr or DSP3 were all performed according to standard procedures provided by the vector supplier.

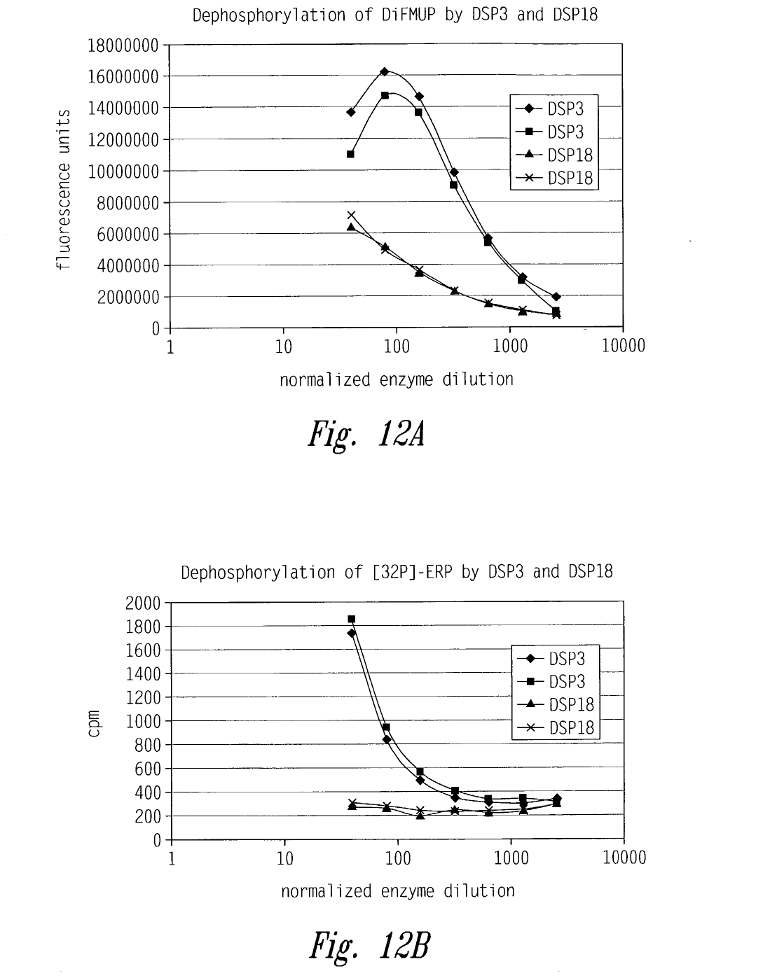

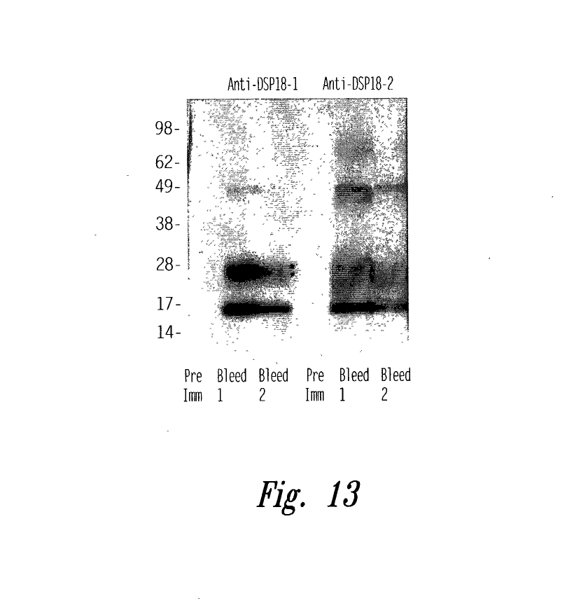

The concentrations of purified DSP-18pr and DSP-3 were determined by the Bio-Rad Protein Assay performed according to the manufacturer's instructions (Bio-Rad, Hercules, Calif.). Following adjustment of the concentration of DSP-3 by diluting it 1:30 in assay buffer (25 mM Tris, pH7.5; 1 mM EDTA, 0.3 mg/ml ovalbumin, and 1 mM DTT) to equal the concentration of DSP-18pr, both samples were titrated in assay buffer in serial two-fold dilutions beginning at a dilution of 1:20. Approximately 30 μl of each diluted enzyme were incubated with 30 μl of 0.6 uM DiFMUP (substrate) for 13 minutes at room temperature. Reactions were terminated by adding 60 μl 2 mM polyphosphate, and fluorescence intensity was measured as described in Example 1. FIG. 12A shows the fluorescence units measured at each dilution for duplicate samples of DSP-18pr and DSP-3.

Phosphatase activity of each enzyme was also measured using as substrate an EGF receptor peptide (D-A-D-E-PY-L-NH₂[SEQ ID NO:35]) (ERP) that corresponds to residues 988-993 of the human EGF receptor. This peptide is available commercially (Bachem Bioscience Inc., King of Prussia, Pa.) and also can be readily synthesized according to established methodologies. ERP substrate was phosphorylated with [γ-³²P]ATP according to the method described by Flint et al. (EMBO J. 12:1937-46 (1993)). Serial dilutions of purified DSP-3 and DSP18pr were prepared as described above and incubated with 30 μl 0.2 μM ³²P-ERP. Reactions were terminated by adding 140 μl activated charcoal, which was then removed by centrifugation. The radioactivity present in supernatants (100 μl) from each sample was measured with a Wallac MicroBeta scintillation counter (Perkin-Elmer Life Sciences, Boston, Mass. FIG. 12B illustrates the phosphatase activities of duplicate samples in DSP-18pr and DSP-3.

### Example 6

### Anti-Peptide Antibodies Specific for DSP-18

This Example describes preparation of anti-DSP-18 peptide antibodies.

Immunization of rabbits and preparation of affinity purified rabbit IgG were performed by ProSci, Inc. (Poway, Calif.) according to the vendor's standard protocol. Briefly, rabbits were immunized with either peptide DSP-18-1 (IDAKDLDQLGR) (SEQ ID NO:36) or peptide DSP-18-2 (VADTPEVPIKK) (SEQ ID NO:37) (two rabbits per peptide) in Freund's complete adjuvant. Animals received a first boost 3 weeks later (Week 3) and a second boost after another 3 weeks (Week 6) with the respective peptide in incomplete Freund's adjuvant. Blood was collected from the rabbits at Day 0 prior to immunization (pre-immune sera) and at Week 7 (Bleed 1) and Week 8 (Bleed 2). Sera collected from each pair of rabbits at each time point were pooled and affinity purified using peptide affinity columns.

The specificity of each anti-peptide rabbit antibody was demonstrated by immunoblotting. Purified DSP-18pr (2 μg) was applied to a preparative 10% polyacrylamide NuPAGE® gel (Invitrogen). After electrophoresis, the separated proteins were electrophorectically transferred from the gel onto an Immobilon-P polyvinylidene fluoride (PVDF) membrane (Millipore). The PVDF membrane was blocked in 5% milk in TBST (20 mM Tris pH 7.5, 150 mM NaCl, 0.05% Tween-20), and cut into 6 strips. Each strip was incubated in one of the following antibodies: (1) pre-immune sera from rabbits immunized with DSP-18-1; (2) purified rabbit anti-DSP-18-1 from Bleed 1 (2 mg/ml); (3) purified rabbit anti-DSP-18-1 from Bleed 2 (2 mg/ml); (4) pre-immune sera from rabbits immunized with DSP-18-2; (5) purified rabbit anti-DSP-18-2 from Bleed 1 (2 mg/ml); (6) purified rabbit anti-DSP-18-2 from Bleed 2 (2 mg/ml) for 1 hour at room temperature, washed 3×10 min with TBST, and then incubated with horseradish peroxidase (HRP) conjugated goat anti-rabbit IgG (1:10,000) (Amersham Biosciences, Piscataway, N.J.) for 30 min at room temperature. Binding was detected with the Western Lightning Chemiluminescent reagent used according to the manufacturer's instructions (Perkin-Elmer Life Sciences) as shown in FIG. 12.

From the foregoing, it will be appreciated that, although specific embodiments of the invention have been described herein for the purpose of illustration, various modifications may be made without deviating from the spirit and scope of the invention. Accordingly, the present invention is not limited except as by the appended claims.

1. An isolated DSP-18 polypeptide comprising a DSP-18a amino acid sequence of DSP-18a as set forth in SEQ ID NO:2, or a variant thereof that differs in one or more amino acid deletions, additions, insertions, or substitutions at no more than 15% of the amino acids in SEQ ID NO:2 such that the polypeptide retains the ability to dephosphorylate an activated MAP-kinase.

2. An isolated DSP-18 polypeptide comprising a DSP-18b amino acid sequence as set forth in SEQ ID NO:4, or a variant thereof that differs in one or more amino acid deletions, additions, insertions, or substitutions at no more than 25% of the amino acids in SEQ ID NO:4 such that the polypeptide retains the ability to dephosphorylate an activated MAP-kinase.

3. An isolated DSP-18 polypeptide comprising a DSP-18c amino acid sequence as set forth in SEQ ID NO:6, or a variant thereof that differs in one or more amino acid deletions, additions, insertions, or substitutions at no more than 15% of the amino acids in SEQ ID NO:6 such that the polypeptide retains the ability to dephosphorylate an activated MAP-kinase.

4. An isolated DSP-18 polypeptide comprising a DSP-18d amino acid sequence as set forth in SEQ ID NO:8, or a variant thereof that differs in one or more amino acid deletions, additions, insertions, or substitutions at no more than 15% of the amino acids in SEQ ID NO:8 such that the polypeptide retains the ability to dephosphorylate an activated MAP-kinase.

5. An isolated DSP-18 polypeptide comprising a DSP-18e amino acid sequence as set forth in SEQ ID NO:10, or a variant thereof that differs in one or more amino acid deletions, additions, insertions, or substitutions at no more than 12% of the amino acids in SEQ ID NO:10 such that the polypeptide retains the ability to dephosphorylate an activated MAP-kinase.

6. An isolated DSP-18 polypeptide comprising a DSP-18f amino acid sequence as set forth in SEQ ID NO:12, or a variant thereof that differs in one or more amino acid deletions, additions, insertions, or substitutions at no more than 5% of the amino acids in SEQ ID NO:12 such that the polypeptide retains the ability to dephosphorylate an activated MAP-kinase.

7. An isolated prototypical DSP-18 polypeptide consisting of a DSP-18pr amino acid sequence as set forth in SEQ ID NO:14 such that the polypeptide retains the ability to dephosphorylate an activated MAP-kinase.

8. An isolated polynucleotide that encodes at least 147 consecutive amino acids of a polypeptide having a sequence corresponding to any one of the sequences selected from the group consisting of SEQ ID NOS:2, 4, 6, 8, and 12.

9. An isolated polynucleotide that encodes at least 136 consecutive amino acids of a polypeptide having an amino acid sequence corresponding to the sequence set forth in SEQ ID NO: 10.

10. An expression vector comprising a polynucleotide according to claim 8 or 9.

11. A host cell transformed or transfected with an expression vector according to claim 10.

12. An isolated polynucleotide that encodes a DSP-18 polypeptide according to any one of claims 1-7.

13. An isolated polynucleotide comprising a nucleotide sequence set forth in any one of the sequences selected from the group consisting of SEQ ID NOS:1, 3, 5, 7, 9, 11, and 13.

14. An expression vector comprising a polynucleotide according to either claim 12 or claim 13.

15. A host cell transformed or transfected with an expression vector according to claim 14.

16. An isolated antisense polynucleotide comprising at least 15 consecutive nucleotides complementary to a polynucleotide according to claim 12.

17. An isolated DSP-18 polynucleotide selected from the group consisting of (i) a polynucleotide that detectably hybridizes to a polynucleotide having a sequence that is complementary to positions 553-660 as set forth in SEQ ID NO:1 under conditions that include a wash in 0.1×SSC and 0.1% SDS at 50° C. for 15 minutes; (ii) a polynucleotide that detectably hybridizes to a polynucleotide having a sequence that is complementary to positions 553-1011 as set forth in SEQ ID NO:3 under conditions that include a wash in 0.1×SSC and 0.1% SDS at 50° C. for 15 minutes; (iii) a polynucleotide that detectably hybridizes to a polynucleotide having a sequence that is complementary to positions 553-660 as set forth in SEQ ID NO:5 under conditions that include a wash in 0.1×SSC and 0.1% SDS at 50° C. for 15 minutes; (iv) a polynucleotide that detectably hybridizes to a polynucleotide having a sequence that is complementary to positions 553-660 as set forth in SEQ ID NO:7 under conditions that include a wash in 0.1×SSC and 0.1% SDS at 50° C. for 15 minutes; (v) a polynucleotide that detectably hybridizes to a polynucleotide having a sequence that is complementary to positions 523-594 as set forth in SEQ ID NO:9 under conditions that include a wash in 0.1×SSC and 0.1% SDS at 50 ° C. for 15 minutes; and (vi) a polynucleotide that detectably hybridizes to a polynucleotide having a sequence that is complementary to positions 553-579 as set forth in SEQ ID NO:11 under conditions that include a wash in 0.1×SSC and 0.1% SDS at 50° C. for 15 minutes.

18. An expression vector comprising a polynucleotide according to either claim 16 or claim 17.

19. A host cell transformed or transfected with an expression vector according to claim 18.

20. A method of producing a DSP-18 polypeptide, comprising the steps of:
(a) culturing a host cell according to claim 15 under conditions that permit expression of a DSP-18 polypeptide; and
(b) isolating the DSP-18 polypeptide from the host cell culture.

21. An isolated antibody, or antigen binding fragment thereof, that specifically binds to a DSP-18 polypeptide having an amino acid sequence set forth in any one of the sequences selected from the group consisting of SEQ ID NOS:2, 4, 6, 8, 10, and 12, wherein the antibody or antigen binding fragment thereof does not specifically bind to a SPG008 polypeptide having the sequence set forth in SEQ ID NO:31 or to a 69109 polypeptide having the amino acid sequence set forth in SEQ ID NO:32 or SEQ ID NO:33.

22. An isolated antibody that specifically binds to an antigenic determinant of a DSP-18 polypeptide having an amino acid sequence selected from the group consisting of the sequence set forth in any one of SEQ ID NO:2, SEQ ID NO:6, and SEQ ID NO:8, wherein the antigenic determinant comprises at least one amino acid located at positions 146-181 of SEQ ID NO:2, SEQ ID NO:6, or SEQ ID NO:8.

23. An isolated antibody that specifically binds to an antigenic determinant of a DSP-18 polypeptide having an amino acid sequence as set forth in SEQ ID NO:4, wherein the antigenic determinant comprises at least one amino acid located at positions 146-298 in SEQ ID NO:4.

24. An isolated antibody that specifically binds to an antigenic determinant of a DSP-18 polypeptide having an amino acid sequence as set forth in SEQ ID NO:10, wherein the antigenic determinant comprises at least one amino acid located at positions 136-159 of SEQ ID NO:10.

25. An isolated antibody that specifically binds to an antigenic determinant of a DSP-18 polypeptide having an amino acid sequence as set forth in SEQ ID NO:12, wherein the antigenic determinant comprises at least one amino acid located at positions 146-154 of SEQ ID NO:12.

26. An isolated antibody that specifically binds to an antigenic determinant of a DSP-18 polypeptide having an amino acid sequence selected from the group consisting of SEQ ID NOS:2, 4, 6, 8, 10, 12, and 14, wherein the antigenic determinant comprises at least one amino acid located at positions 18-28 of any one of SEQ ID NOS: 2, 4, 6, 8, 10, 12, and 14.

27. An isolated antibody that specifically binds to an antigenic determinant of a DSP-18 polypeptide having an amino acid sequence selected from the group consisting of SEQ ID NOS:2, 4, 6, 8, 10, 12, and 14, wherein the antigenic determinant comprises at least one amino acid located at positions 55-65 of any one of SEQ ID NOs: 2, 4, 6, 8, 10, 12, and 14.

28. An antibody or fragment thereof according to any one of claims 21-27, wherein the antibody is a monoclonal antibody.

29. A pharmaceutical composition comprising an antibody or fragment thereof according to any one of claims 21-27 in combination with a physiologically acceptable carrier.

30. A method for detecting the presence of a DSP-18 polypeptide in a sample, comprising:
(a) contacting a sample with an antibody according to any one of claims 21-27, or an antigen-binding fragment thereof according to any one of claims 21-27, under conditions and for a time sufficient to allow formation of an antibody/DSP-18 polypeptide complex; and
(b) detecting a level of antibody/DSP-18 polypeptide complex, and therefrom detecting the presence of DSP-18 polypeptide in the sample.

31. A method according to claim 30, wherein the antibody is linked to a support material.

32. A method according to claim 30, wherein the antibody is linked to a detectable marker.

33. A method according to claim 30, wherein the sample is a biological sample obtained from a patient.

34. A method for detecting DSP-18 expression in a sample, comprising:
(a) contacting a sample with either an isolated antisense polynucleotide according to claim 16 or an isolated DSP-18 polynucleotide according to claim 17; and
(b) detecting presence in the sample of a polynucleotide that hybridizes to the isolated antisense polynucleotide of claim 16 or to the isolated DSP-18 polynucleotide of claim 17, and therefrom detecting DSP-18 expression in the sample.

35. A method according to claim 34, wherein the amount of detected polynucleotide that hybridizes to the isolated antisense polynucleotide or to the DSP-18 isolated polynucleotide is determined using polymerase chain reaction.

36. A method according to claim 34, wherein the amount of detected polynucleotide that hybridizes to the isolated antisense polynucleotide or to the DSP-18 isolated polynucleotide is determined using a hybridization assay.

37. A method according to claim 34, wherein the sample comprises an RNA or cDNA preparation.

38. A method of screening for an agent that modulates DSP-18 activity, comprising the steps of:
(a) contacting a candidate agent with a polypeptide according to any one of claims 1-7, under conditions and for a time sufficient to permit interaction between the polypeptide and candidate agent; and
(b) subsequently evaluating the ability of the polypeptide to dephosphorylate a DSP-18 substrate, relative to a predetermined ability of the polypeptide to dephosphorylate the DSP-18 substrate in the absence of candidate agent;
and therefrom identifying an agent that modulates DSP-18 activity.

39. A method according to claim 38, wherein the DSP-18 substrate is a MAP-kinase.

40. A method according to claim 38, wherein the candidate agent is a small molecule.

41. A method according to claim 40, wherein the small molecule is present within a combinatorial library.

42. A method of screening for an agent that modulates DSP-18 activity, comprising the steps of:
(a) contacting a candidate agent with a cell comprising a DSP-18 promoter operably linked to a polynucleotide encoding a detectable transcript or protein, under conditions and for a time sufficient to permit interaction between the promoter and candidate agent; and
(b) subsequently evaluating the expression of the polynucleotide, relative to a predetermined level of expression in the absence of candidate agent;
and therefrom identifying an agent that modulates DSP-18 activity.

43. A method according to claim 42, wherein the polynucleotide encodes a DSP-18 polypeptide.

44. A method according to claim 42, wherein the polynucleotide encodes a reporter protein.

45. A method for modulating a proliferative response in a cell, comprising contacting a cell with an agent that modulates DSP-18 activity.

46. A method for modulating differentiation of a cell, comprising contacting a cell with an agent that modulates DSP-18 activity.

47. A method for modulating survival of a cell, comprising contacting a cell with an agent that modulates DSP-18 activity.

48. A method according to any one of claims 45-47, wherein the agent modulates a pattern of gene expression.

49. A method according to any one of claims 45-47, wherein the cell displays contact inhibition of cell growth.

50. A method according to any one of claims 45-47, wherein the cell displays anchorage independent growth.

51. A method according to any one of claims 45-47 wherein the cell displays an altered intercellular adhesion property.

52. A method according to claim 47, wherein the agent modulates apoptosis.

53. A method according to claim 47, wherein the agent modulates the cell cycle.

54. A method according to claim 44, wherein the cell is present within a patient.

55. A method for treating a patient afflicted with a disorder associated with DSP-18 activity, comprising administering to a patient a therapeutically effective amount of an agent that modulates DSP-18 activity.

56. A method according to claim 55, wherein the disorder is selected from the group consisting of Duchenne muscular dystrophy, cancer, graft-versus-host disease, autoimmune diseases, allergies, metabolic diseases, abnormal cell growth, abnormal cell proliferation and cell cycle abnormalities.

57. A DSP-18a substrate trapping mutant polypeptide that differs from an amino acid sequence set forth in SEQ ID NO:2, or a variant thereof in one or more amino acid deletions, additions, insertions, or substitutions at no more than 15% of the amino acids in SEQ ID NO:2, such that the polypeptide binds to a substrate with an affinity that is not substantially diminished relative to DSP-18a, and such that the ability of the polypeptide to dephosphorylate a substrate is reduced relative to DSP-18a.

58. A DSP-18b substrate trapping mutant polypeptide that differs from an amino acid sequence set forth in SEQ ID NO:4, or a variant thereof in one or more amino acid deletions, additions, insertions, or substitutions at no more than 25% of the amino acids in SEQ ID NO:4, such that the polypeptide binds to a substrate with an affinity that is not substantially diminished relative to DSP-18b, and such that the ability of the polypeptide to dephosphorylate a substrate is reduced relative to DSP-18b.

59. A DSP-18c substrate trapping mutant polypeptide that differs from an amino acid sequence set forth in SEQ ID NO:6, or a variant thereof in one or more amino acid deletions, additions, insertions, or substitutions at no more than 15% of the amino acids in SEQ ID NO:6, such that the polypeptide binds to a substrate with an affinity that is not substantially diminished relative to DSP-18c, and such that the ability of the polypeptide to dephosphorylate a substrate is reduced relative to DSP-18c.

60. A DSP-18d substrate trapping mutant polypeptide that differs from an amino acid sequence set forth in SEQ ID NO:8, or a variant thereof in one or more amino acid deletions, additions, insertions, or substitutions at no more than 15% of the amino acids in SEQ ID NO:8, such that the polypeptide binds to a substrate with an affinity that is not substantially diminished relative to DSP-18d, and such that the ability of the polypeptide to dephosphorylate a substrate is reduced relative to DSP-18d.

61. A DSP-18e substrate trapping mutant polypeptide that differs from an amino acid sequence set forth in SEQ ID NO:10, or a variant thereof in one or more amino acid deletions, additions, insertions, or substitutions at no more than 12% of the amino acids in SEQ ID NO:10, such that the polypeptide binds to a substrate with an affinity that is not substantially diminished relative to DSP-18e, and such that the ability of the polypeptide to dephosphorylate a substrate is reduced relative to DSP-18e.

62. A DSP-18f substrate trapping mutant polypeptide that differs from an amino acid sequence set forth in SEQ ID NO:12, or a variant thereof in one or more amino acid deletions, additions, insertions, or substitutions at no more than 5% of the amino acids in SEQ ID NO:12, such that the polypeptide binds to a substrate with an affinity that is not substantially diminished relative to DSP-18f, and such that the ability of the polypeptide to dephosphorylate a substrate is reduced relative to DSP-18f.

63. A DSP-18pr substrate trapping mutant polypeptide that differs from an amino acid sequence set forth in SEQ ID NO:14, or a variant thereof in one or more amino acid deletions, additions, insertions, or substitutions at no more than 20% of the amino acids in SEQ ID NO:14, such that the polypeptide binds to a substrate with an affinity that is not substantially diminished relative to DSP-18pr, and such that the ability of the polypeptide to dephosphorylate a substrate is reduced relative to DSP-18pr, wherein the DSP-18pr substrate trapping mutant polypeptide does not consist of an amino acid sequence set forth in SEQ ID NOS:31-33.

64. A substrate trapping mutant polypeptide according to any one of claims 57-63, wherein the polypeptide contains a substitution at position 72 or at position 103 of any one of the sequences selected from the group consisting of SEQ ID NOS:2, 4, 6, 8, 10, 12, and 14.

65. A method of screening for a molecule that interacts with a DSP-18 polypeptide, comprising the steps of:
(a) contacting a candidate molecule with an isolated DSP-18 polypeptide according to any one of claims 1-7 under conditions and for a time sufficient to permit the candidate molecule and the DSP-18 polypeptide to interact; and
(b) detecting presence or absence of binding of the candidate molecule to the DSP-18 polypeptide, and therefrom determining whether the candidate molecule interacts with DSP-18.

66. A method according to claim 65, wherein the step of detecting comprises an affinity purification step.

67. A method according to claim 65, wherein the step of detecting comprises a yeast two-hybrid screen or a phage display library screen.

68. An immunogen comprising a DSP-18 peptide comprising an amino acid sequence of at least ten consecutive amino acids of a polypeptide selected from the group consisting of DSP-18a as set forth in SEQ ID NO:2, DSP-18b as set forth in SEQ ID NO:4, DSP-18c polypeptide as set forth in SEQ ID NO:6, DSP-18d polypeptide as set forth SEQ ID NO:8, DSP-18e polypeptide as set forth in SEQ ID NO:10, DSP-18f polypeptide as set forth in SEQ ID NO:12, and DSP-18pr polypeptide as set forth in SEQ ID NO:14.

69. The immunogen of claim 68 wherein the DSP-18 peptide comprises an amino acid sequence as set forth in SEQ ID NO:36.

70. The immunogen of claim 68 wherein the DSP-18 peptide comprises an amino acid sequence as set forth in SEQ ID NO:37.

71. An immunogen comprising a DSP-18 peptide comprising an amino acid sequence of at least four consecutive amino acids selected from the group consisting of amino acids at positions 146-181 of a DSP-18a polypeptide as set forth in SEQ ID NO:2, amino acids at positions 146-298 of SEQ ID NO:4, amino acids at positions 146-181 of a DSP-18c polypeptide as set forth in SEQ ID NO:6, amino acids at positions 146-181 of a DSP-18d polypeptide as set forth in SEQ ID NO:8, amino acids at positions 136-159 of a DSP-18e polypeptide as set forth in SEQ ID NO: 10, and amino acids at positions 146-154 of DSP-18f as set forth in SEQ ID NO:12.

72. An immunogen comprising a DSP-18 peptide comprising an amino acid sequence of at least 11 consecutive amino acids selected from the group consisting of amino acids at positions 136-181 of a DSP-18a polypeptide as set forth in SEQ ID NO:2, amino acids at positions 136-298 of a DSP-18b polypeptide as set forth in SEQ ID NO:4, amino acids at positions 136-181 of a DSP-18c polypeptide as set forth in SEQ ID NO:6, and amino acids at positions 136-181 of a DSP-18d polypeptide as set forth in SEQ ID NO: 8.
